(19) United States
(12) Patent Application Publication
(10) Pub. No.: US 2024/0009121 A1
(43) Pub. Date: Jan. 11, 2024

(54) PHARMACEUTICAL COMPOSITION CONTAINING NEUROACTIVE STEROID AND USE THEREOF

(71) Applicant: Brii Biosciences, Inc., Durham, NC (US)

(72) Inventors: Zhi HONG, Durham, NC (US); John KRAUS, Pittsboro, NC (US); Lianhong XU, Palo Alto, CA (US); Jean-Luc GIRARDET, San Diego, CA (US); Ji MA, Foster City, CA (US)

(21) Appl. No.: 18/041,948

(22) PCT Filed: Aug. 17, 2021
(86) PCT No.: PCT/US2021/046347

§ 371 Date: Feb. 16, 2023

Related U.S. Application Data
(60) Provisional application No. 63/066,808, filed on Aug. 17, 2020.
(60) Provisional application No. 63/109,847, filed on Nov. 4, 2020.

(51) Int. Cl. A61K 9/10; A61K 31/57; A61K 47/26; A61K 47/12; A61K 47/10; A61P 25/24

(52) U.S. Cl. CPC A61K 9/10; A61K 31/57; A61K 47/26; A61K 47/12; A61K 47/10; A61P 25/24

(57) ABSTRACT

Disclosed herein is a method and an extended-release aqueous suspension pharmaceutical composition and its use thereof.

### CROSS REFERENCE TO RELATED APPLICATIONS

This application claims the benefit of and priority to U.S. Provisional Application Ser. No. 63/066,808, filed Aug. 17, 2020, and U.S. Provisional Application Ser. No. 63/109,847, filed Nov. 4, 2020, each of which is herein incorporated by reference in its entirety.

### BACKGROUND

Neurological conditions such as postpartum depression (PPD) are serious illnesses. For example, the prevalence of PPD is estimated to be 10-15% depending on methodology and can be as high as 30%. Approximately 40% of women will report their first episode of depression during the postpartum period. In a study of 209 women referred for major depression during or after pregnancy 11.5% reported start of depression during pregnancy, 66.5% reported start of depression within 6 weeks after childbirth (early postpartum), and 22% reported onset 6 weeks after childbirth (late postpartum). Some may have onset of depression at more than 27 weeks after childbirth (Cox J L, et al., Br J Psychiatry. p: 163:27-31, July 1993).

It is generally believed that rapid changes in allopregnanolone levels after child birth may contribute to the etiology of PPD in vulnerable women. Allopregnanolone (ALLO) is a neuroactive steroid (NAS) that acts as a positive allosteric modulator (PAM) of GABA action on the γ aminobutyric acid type A receptors (GABA_{A}). ALLO prolongs the opening time of the GABA_{A} chloride channel, enhancing inhibitory neurotransmission. Pregnancy is associated with greatly elevated levels of female sex hormones, including progesterone, accompanied by increased levels of ALLO in both the blood and brain. Shortly after giving birth, both progesterone and ALLO levels drop precipitously and it has been hypothesized that this abrupt drop may trigger PPD in vulnerable women (Schiller, et al., Psychopharmacology (Berl). 231(17): 3557-3567, 2014, doi:10.1007/s00213-014-3599-x; McEvoy, et al., Curr Psychiatry Rep 20:78, 2018, https://doi.org/10.1007/s11920-018-0937-4 2018).

Brexanolone intravenous injection product (brexanolone IV, ZULRESSO™, was approved by US Food and Drug Administration in 2019 for the treatment of postpartum depression (PPD). However, ZULRESSO™ is inconvenient to use and is administered to a patient by continuous intravenous (IV) infusion that lasts for a total of about 60 hours (2.5 days). There are significant limitations on ZULRESSO™ use: complexity of infusion protocol (continuous infusion over 60 hours with multiple bag changes in a hospital), patient inconvenience (tethered during a long infusion with requirement for clinical supervision during infusion), and significant safety issue such as loss of consciousness and adverse effect (AE). The safety profile of ZULRESSO™ has resulted in an onerous Risk Evaluation and Mitigation Strategy (REMS) that requires, briefly, that ZULRESSO™ be administered to patients in a medically supervised setting that provides monitoring during administration; only certified pharmacies and healthcare settings can dispense ZULRESSO™; patients must be educated on the risks of serious harm from excessive sedation and loss of consciousness and the need for monitoring while ZULRESSO™ is administered; and patients must be enrolled in a registry.

Therefore, there is an unmet need for a better treatment option, such as an injection dosage form of brexanolone, that can overcome the limitations of the existing product.

### SUMMARY

Disclosed herein is a novel pharmaceutical composition comprising a neuroactive steroid and its use thereof. In one aspect, disclosed is an aqueous suspension pharmaceutical composition comprising a pharmaceutically effective amount of a neuroactive steroid, wherein the neuroactive steroid provides a therapeutically effective plasma concentration over a period of at least about 24 hours, 48 hours, 72 hours, 96 hours, 120 hours, 144 hours, 168 hours, 192 hours, 216 hours, 240 hours, or 336 hours to treat a neurological condition when administered in one or more injections to a subject in need thereof. In some cases, the neuroactive steroid is selected from the group consisting of tetrahydrodeoxycorticosterone (THDOC), androstane, androstane 3α-androstanediol, cholestane cholesterol, pregnane, eltanolone, brexanolone, ganaxolone, zuranolone, or a combination thereof. In some cases, the neuroactive steroid is selected from the group consisting of brexanolone, pharmaceutically acceptable salts and derivatives thereof. In some cases, the neuroactive steroid comprises brexanolone. In some cases, the at least about 24 hours, 48 hours, 72 hours, 96 hours, 120 hours, 144 hours, 168 hours, 192 hours, 216 hours, 240 hours, or 336 hours are the hours after the initial administration of the one or more injections, for example, at least about 72 hours after the initial administration of the one or more injections. In some cases, the at least about 24 hours, 48 hours, 72 hours, 96 hours, 120 hours, 144 hours, 168 hours, 192 hours, 216 hours, 240 hours, or 336 hours are the hours after the neuroactive steroid reaches the therapeutically effective plasma concentration, for example, at least about 72 hours after the neuroactive steroid reaches the therapeutically effective plasma concentration.

In some cases, the aqueous suspension pharmaceutical composition comprises from 30 mg to 1000 mg of brexanolone. In some cases, the concentration of brexanolone is from about 30 mg/mL to about 500 mg/mL. In some cases, the brexanolone has a particle size distribution (PSD) with a Dv50 of from about 1 μm to about 5 μm. In some cases, the brexanolone has a particle size distribution (PSD) with a Dv50 of about 3 μm. In some cases, the brexanolone has a particle size distribution (PSD) with a Dv90 of from about 4 μm to about 8 μm. In some cases, the brexanolone has a particle size distribution (PSD) with a Dv90 of about 6 μm.

In some cases, the aqueous suspension pharmaceutical composition further comprises one or more pharmaceutically acceptable excipients. In some cases, the one or more pharmaceutically acceptable excipients comprises a surfactant, a buffering agent, or both. In some cases, the surfactant is a nonionic surfactant. In some cases, the surfactant comprises polysorbate 80. In some cases, the surfactant comprises about 0.2% to about 1.0% w/v of the composition. In some cases, the surfactant comprises about 0.5% to about 0.9% w/v of the composition. In some cases, the surfactant comprises about 0.6% to about 0.8% w/v of the composition. In some cases, the buffering agent comprises about 0.1% to about 0.5% w/v of the composition. In some cases, the buffering agent comprises a citrate buffering agent. In some cases, the citrate buffering agent comprises sodium citrate dihydrate and citric acid monohydrate. In some cases, the sodium citrate dihydrate is about 0.15% to about 0.2% w/v of the composition. In some cases, the citric acid monohydrate is about 0.010% to about 0.015% w/v of the composition. In some cases, the aqueous suspension pharmaceutical composition further comprises a suspending agent. In some cases, the suspending agent comprises polyethylene glycol (PEG). In some cases, the PEG is a higher molecular weight PEG. In some cases, the higher molecular weight PEG is PEG 3350, PEG 4000 or PEG 6000. In some cases, the higher molecular weight PEG is PEG 3350. In some cases, the suspending agent comprises about 0.2% to about 1.0% w/v of the composition. In some cases, the suspending agent comprises about 0.5% to about 0.9% w/v of the composition. In some cases, the suspending agent comprises about 0.6% to about 0.8% w/v of the composition. In some cases, the aqueous suspension pharmaceutical composition further comprises a tonicity adjusting agent. In some cases, the tonicity adjusting agent is selected from the group consisting of dextrose, mannitol and glycerin. In some cases, the tonicity adjusting agent is mannitol. In some cases, the tonicity adjusting agent comprises about 2% to about 6% w/v of the pharmaceutical composition. In some cases, the tonicity adjusting agent comprises about 3% to about 4% w/v of the pharmaceutical composition.

In some cases, the pharmaceutical composition is substantially free of cyclodextrins. In some cases, the aqueous suspension pharmaceutical composition is substantially free of sulfobutyl ether β-cyclodextrin. In some cases, the pharmaceutical composition is substantially free of hydroxypropyl-β-cyclodextrin (HPBCD).

In some cases, the neuroactive steroid comprises a brexanolone crystalline form (polymorph Form A) characterized by having at least 2 of the following peaks in Powder X-Ray Diffraction (PXRD) diffractograms, at 7.25, 8.88, 11.46, 14.50, 14.78, 17.77, 18.15, 18.32, 18.61 and 19.99±0.1 2θ(°).

In some cases, the pharmaceutical composition provides a maximum blood plasma concentration (Cmax) of more than about 10 ng/mL brexanolone following the one or more injections. In some cases, the maximum blood plasma concentration (Cmax) of brexanolone ranges from about 20 ng/mL to about 80 ng/mL following the one or more injections. In some cases, the maximum blood plasma concentration (Cmax) of brexanolone is about 50 ng/mL following the one or more injections. In some cases, the maximum blood plasma concentration (Cmax) of brexanolone following the one or more injections is less than 90% of the Cmax of a reference product administered via IV infusion containing substantially the same amount of brexanolone. In some cases, at least about 50% of the maximum blood plasma concentration (Cmax) is maintained for a period greater than about 50 hours following the one or more injections. In some cases, at least about 40% of the maximum blood plasma concentration (Cmax) is maintained for a period greater than about 100 hours following the one or more injections. In some cases, at least about 30% of the maximum blood plasma concentration (Cmax) is maintained for a period greater than about 300 hours following the one or more injections. In some cases, the pharmaceutical composition provides a mean steady state exposure (Css) of brexanolone following the one or more injections within the range of about 80% to about 125% of the mean steady state exposure of a reference product administered via IV infusion containing substantially the same amount of brexanolone. In some cases, the pharmaceutical composition provides a mean steady state exposure of brexanolone within the range of about 80% to about 125% of 52 ng/mL to about 79 ng/mL following the one or more injections. In some cases, the pharmaceutical composition provides an average daily AUC of brexanolone that is at least about 50 ng*h/mL/day for at least about 72 hours following the one or more injections. In some cases, the composition achieves a mean terminal elimination half-life (T½) of brexanolone of greater than about 9 h following the one or more injections. In some cases, the composition achieves a mean terminal elimination half-life (T½) of brexanolone that is greater than the T½ of a reference product administered via IV infusion containing substantially the same amount of brexanolone.

In another aspect, disclosed is a method, comprising administering to a subject in need thereof a therapeutically effective dose of the pharmaceutical composition disclosed herein. In another aspect, disclosed is a method of treating or preventing a neurological condition in a subject in need thereof, comprising administering to the subject a therapeutically effective dose of the pharmaceutical composition disclosed herein. In another aspect, disclosed is a method of treating or preventing a neurological condition in a subject in need thereof, comprising administering to the subject an aqueous suspension pharmaceutical composition comprising a pharmaceutically effective amount of a neuroactive steroid, wherein the neuroactive steroid provides a therapeutically effective plasma concentration over a period of at least about 72 hours. In some cases, the neuroactive steroid is selected from the group consisting of tetrahydrodeoxycorticosterone (THDOC), androstane, androstane 3α-androstanediol, cholestane cholesterol, pregnane, eltanolone, brexanolone, ganaxolone, zuranolone, or a combination thereof. In some cases, the neuroactive steroid is selected from the group consisting of brexanolone, pharmaceutically acceptable salts and derivatives thereof. In some cases, the neuroactive steroid comprises brexanolone.

In some cases, the pharmaceutical composition is administered to the subject between a pre-admin breastfeeding and a consecutive post-admin breastfeeding of the subject. In some cases, the pharmaceutical composition is administered to the subject from 1 minute to about 360 minutes after completion of the pre-admin breastfeeding. In some cases, the pharmaceutical composition is administered to the subject about 5 minutes to about 360 minutes before starting the post-admin breastfeeding. In some cases, the subject is a woman 1 day to 12 months after giving birth to a child. In some cases, the subject has not been diagnosed with the neurological condition at the time of administering the pharmaceutical composition. In some cases, the subject is diagnosed with the neurological condition within 2 years prior to administering the pharmaceutical composition. In some cases, the subject is diagnosed with the neurological condition during pregnancy prior to administering the pharmaceutical composition. In some cases, the subject has a family history of the neurological condition at the time of administering the pharmaceutical composition.

In some cases, the neurological condition is selected from the group consisting of traumatic brain injury, Alzheimer's disease, mild cognitive impairment (MCI), epilepsy, seizures, anxiety, fragile X tremor-ataxia syndrome, lysosomal storage disorders (Niemann-Pick type C disease), post-traumatic stress disorder (PTSD), postpartum depression (PPD), major depressive disorder (MDD), premenstrual dysphoric disorder (PMDD), persistent depressive disorder (PDD), bipolar disorder, seasonal affective disorder (SAD), secondary depression, postfinasteride syndrome, alcohol craving, and smoking cessation. In some cases, the neurological condition is postpartum depression (PPD). In some cases, the pharmaceutical composition is administered to the subject via intramuscular (IM) injection.

In some cases, the subject experiences a reduction of depression that is characterized by at least a four point decline in total Hamilton Depression Rating Scale (HAM-D) value or by at least a two point decline in Montgomery Åsberg Depression Rating Scale (MADRS) value, within two months after administering an initial dose of the pharmaceutical composition. In some cases, the subject experiences a reduction of depression that is characterized by at least a 40% reduction in HAM-D or MADRS value, within two months after administering an initial dose of the pharmaceutical composition. In some cases, the subject experiences a reduction of depression that is characterized by HAM-D or MADRS remission, within two months after administering an initial dose of the pharmaceutical composition. In some cases, the subject experiences a reduction of depression that is characterized by an at least two category change in HAM-D severity classification, within two months after administering an initial dose of the pharmaceutical composition. In some cases, the subject experiences a reduction of depression that is characterized by at least one point decline in one or more of the Clinical Global Impression (CGI) subscale scores, wherein the CGI subscales are selected from Severity of Illness Subscale (CGI-S) or Global Improvement Subscale (CGI-I), within two months after administering an initial dose of the pharmaceutical composition, within two months after administering an initial dose of the pharmaceutical composition. In some cases, the subject experiences a reduction of depression that is characterized by at least about a 10%, 20%, or 30% improvement in Symptoms of Depression Questionnaire (SDQ) total scale score or in any of the respective subscales of SDQ-1, SDQ-2, SDQ-3, SDQ-4 and SDQ-5, within two months after administering an initial dose of the pharmaceutical composition. In some cases, after administering an initial dose, the subject experiences a reduction of depression that is characterized by an at least one point decline in Pittsburgh Sleep Quality Index (PSQI) Global score.

In some cases, the administering comprises: (a) administering an initial dose of the pharmaceutical composition disclosed herein; and (b) optionally, administering a second dose or subsequent dose of the pharmaceutical composition disclosed herein, wherein the second dose or subsequent doses are administered at a timepoint deemed necessary to maintain a therapeutically effective plasma concentration of brexanolone.

In some cases, the initial dose of brexanolone and subsequent dose(s) are the same. In some cases, the initial dose of brexanolone and subsequent dose(s) are different. In some cases, the initial dose of brexanolone is greater than a subsequent dose. In some cases, the initial dose of brexanolone is less than a subsequent dose.

In another aspect, disclosed herein is a method comprising: 1) obtaining or causing to obtain depression assessment data of the subject, wherein the depression assessment data comprise depression diagnostic data and pregnancy data of the subject; 2) producing risk prediction data based on the depression assessment data; and 3) administering an aqueous suspension pharmaceutical composition comprising a pharmaceutically effective amount of a neuroactive steroid selected from the group consisting of brexanolone, pharmaceutically acceptable salts and derivatives thereof to the subject prior to clinical onset of the PPD if the risk prediction data indicate a high risk of PPD in the subject, wherein the neuroactive steroid provides a therapeutically effective plasma concentration over a period of at least about 24 hours, 48 hours, 72 hours, 96 hours, 120 hours, 144 hours, 168 hours, 192 hours, 216 hours, 240 hours, or 336 hours to treat a neurological condition when administered in one or more injections to a subject in need thereof; and wherein the subject is not diagnosed with PPD at the time the depression assessment data is obtained. In some cases, the aqueous suspension pharmaceutical composition is the aqueous suspension pharmaceutical composition disclosed herein.

In some cases, the depression diagnostic data comprise historic depression diagnostic data if any, depression data from previous pregnancy if any, present depression diagnostic data, historic Beck's Depression Inventory (BDI) value, present BDI value, historic Edinburgh Postnatal Depression Scale (EPDS) value, present EPDS value, historic Postpartum Depression Predictors Inventory (PDPI), present PDPI value, historic SIGH-ADS29 assessment value, present SIGH-ADS29 assessment value, historic Structured Clinical Interview for DSM-IV (SCID) assessment, present SCID assessment, historic Inventory of Depressive Symptomatology (IDS) assessment, present IDS assessment, historic Quick Inventory of Depressive Symptomatology (QIDS) assessment, present QIDS assessment, clinician IDS (IDS-C), clinician QIDS (QIDS-C), patient self-rated IDS (IDS-SR), patient self-rated QIDS (QIDS-SR), of said subject, or a combination thereof. In some cases, the depression assessment data is obtained or caused to be obtained during pregnancy, in a range of from 10 weeks to 0 day prior to the completion of pregnancy, in a range of from 0 day to 24 weeks after completion of pregnancy, of the subject, or a combination thereof. In some cases, the neuroactive steroid is administered to the subject in a range of from 0 day to 24 weeks after completion of pregnancy of the subject.

In another aspect, disclosed herein is the use of the pharmaceutical composition disclosed herein for manufacturing a medicament for treating or preventing a neurological condition. In some cases, the neurological condition is selected from the group consisting of traumatic brain injury, Alzheimer's disease, mild cognitive impairment (MCI), epilepsy, seizures, anxiety, fragile X tremor-ataxia syndrome, lysosomal storage disorders (Niemann-Pick type C disease), post-traumatic stress disorder (PTSD), postpartum depression (PPD), major depressive disorder (MDD), premenstrual dysphoric disorder (PMDD), persistent depressive disorder (PDD), bipolar disorder, seasonal affective disorder (SAD), secondary depression, postfinasteride syndrome, alcohol craving, and smoking cessation. In some cases, the neurological condition is postpartum depression (PPD).

### BRIEF DESCRIPTION OF THE FIGURES

FIG. 1A-FIG. 1D. Schematic illustrations of exemplary particle size distributions. FIG. 1A: a pharmaceutical composition comprises minimum amounts, such as less than 1% of small particles and mostly large particles. FIG. 1B: A pharmaceutical composition comprises some small particles and mostly large particles. FIG. 1C: A pharmaceutical composition comprises increasing amounts of small particles and large particles. FIG. 1D: A pharmaceutical composition comprises comparable amounts of small particles and large particles. D50=mass-median-diameter (MMD), wherein 50% of particles are below and 50% of particles are above a given diameter. Mean Large=mean particle size of the large particles. Mean Small=mean particle size of the small particles.

FIG. 2A-FIG. 2E. Examples of pharmaceutical compositions comprising brexanolone. FIG. 2A: Brexanolone structure. FIG. 2B: Small brexanolone particle size distribution. FIG. 2C: Large brexanolone particle size distribution. FIG. 2D-FIG. 2E: Pharmacokinetics (PK) in rats showing rat plasma brexanolone concentrations over time after administration. Legend: Open diamond, brexanolone suspension of small particle, 25 mg/kg; Open square, brexanolone suspension of large particles, 25 mg/kg; Solid square, IV solution (Comparative) 1 mg/kg; and Solid triangle, IM solution (Comparative) 12.5 mg/kg.

FIG. 3A-FIG. 3E. Examples of pharmaceutical compositions comprising ganaxolone. FIG. 3A: Ganaxolone structure. FIG. 3B: Distribution of 4.1 μm ganaxolone particles. FIG. 3C: Distribution of 3.6 μm ganaxolone particles. FIG. 3D-FIG. 3E: Pharmacokinetics (PK) in rats showing rat plasma concentrations over time after administration. Legend: Open diamond, ganaxolone suspension of 1 μm particles, 25 mg/kg; Open square, ganaxolone suspension of 4 μm particles, 25 mg/kg; Solid square, IV solution (Comparative); and Solid diamond, IM solution (Comparative).

FIG. 4A-FIG. 4B. Examples of Powder X-Ray Diffraction (PXRD) diffractograms. FIG. 4A: Pre-processing commercial brexanolone showing one major peak at position 18.15 2θ. FIG. 4B: Crystalline Form A brexanolone showing multiple peaks at 7.25 and 18.15 2θ.

FIG. 5. A plasma concentration—time curve showing mean±SD brexanolone plasma concentrations in male Sprague Dawley rats following a single I (1.0 mg/kg) dose.

FIG. 6. A plasma concentration—time curve showing mean±SD brexanolone plasma concentration in male Beagle dogs following a single IV (2.0 mg/kg) dose.

FIG. 7. A plasma concentration—time curve showing mean±SD brexanolone plasma concentrations in male Sprague Dawley rats following a single IM dose.

FIG. 8. A plasma concentration—time curve showing mean±SD brexanolone plasma concentrations in Beagle dogs following a single IM dose.

FIG. 9. A schematic diagram showing a dual path absorption two compartment linear population PK model for single intramuscular injection of extended-release injectable suspension formulations of brexanolone.

FIG. 10. A plasma concentration—time curves showing population PK model fit to the PK profiles of intramuscular administration of brexanolone using different extended-release injectable suspension formulations in male Sprague Dawley rats and Beagle dogs.

FIG. 11. Plasma concentration—time curves showing predicted human PK profiles after single intramuscular administration of ER (extended-release) Brexanolone to healthy adult subjects based on dog-like or rat-like absorption kinetic profiles.

FIG. 12. Scheme showing study cohorts dosing schedule for a Phase 1 open-label, Single Ascending Dose (SAD) escalation study.

FIG. 13. A plasma concentration—time curve for Cohort 4 subjects administered 1.0 mL of a 300 mg/mL dose of ER Brexanolone-A (IM).

FIG. 14. Overlaid plasma concentration—time curves for four Cohort 4 subjects (2 male, 2 female) administered 1.0 mL of a 300 mg/mL dose of ER Brexanolone-A (IM).

FIG. 15. Overlaid plasma concentration—time curves comparing brexanolone concentration over time for Cohort 4 subjects administered 1.0 mL of a 300 mg/mL dose of ER Brexanolone-A to Cohort 2 subjects administered 3.0 mL of a 100 mg/mL dose of ER Brexanolone-A.

FIG. 16. A plasma concentration—time curve for Cohort 5 subjects administered 1.0 mL of a 300 mg/mL dose of ER Brexanolone-B (IM).

FIG. 17. Overlaid plasma concentration—time curves for three Cohort 5 subjects administered 3.0 mL of a 100 mg/mL dose of ER Brexanolone-B.

FIG. 18. Overlaid plasma concentration—time curves comparing brexanolone concentration over time for Cohort 5 subjects administered 3.0 mL of a 100 mg/mL dose of ER Brexanolone-B to Cohort 3 subjects administered 1.0 mL of a 100 mg/mL dose of ER Brexanolone-A and Cohort 1 subjects administered 0.3 mL of a 100 mg/mL dose of ER Brexanolone-B.

FIG. 19A. Representative illustrations of one example of treatment schedule; FIG. 19B: Representative illustrations of another example of the treatment schedule.

FIG. 20. Simulated plasma concentration—time curves for a 60-hour IV infusion of brexanolone in human subjects. The graph models two dosages (60 mg and 90 mg) each administered in three phases: Phase 1 (24-h titration phase); Phase 2: 28-h maximum dose phase; Phase 3: tapering phase. The shaded area represents 95% prediction interval and the median (solid line) for each time point in a simulated population of 1000 individuals.

### DEFINITIONS

Following are more detailed descriptions of various concepts related to, and embodiments of, methods and apparatus according to the present disclosure. It should be appreciated that various aspects of the subject matter introduced above and discussed in greater detail below may be implemented in any of numerous ways, as the subject matter is not limited to any particular manner of implementation. Examples of specific implementations and applications are provided primarily for illustrative purposes.

As used herein, the term “γ aminobutyric acid type A receptors”, “GABA_{A} receptors”, “GABA_{A}Rs”, “GABA_{A}R”, “GABAARs”, “GABAAR” or a grammatic variation thereof, either in singular or in plural form, refers to gamma-aminobutyric acid type A receptors (GABA_{A}Rs) that are a class of receptors that respond to the neurotransmitter gamma-aminobutyric acid (GABA). GABA is the principal inhibitory neurotransmitter in the cerebral cortex that is important for maintaining the inhibitory state that counterbalances neuronal excitation. Disorder in GABA_{A} receptors or imbalance of GABA and neuroexcitation can lead to a wide range of brain circuits and disorders related to GABA function that are central to a variety of behavioral states such as anxiety levels, panic, stress response, seizures, sleep, vigilance and memory. A number of natural and synthetic neuroactive steroids can bind to GABA_{A}Rs and modulate their activities.

As used herein, the term “neuroactive steroid”, “NAS”, “neuroactive steroids”, “NASs” or a variation thereof refers to one or more neurosteroids (NS) that exert inhibitory actions on neurotransmission, specifically, on the GABA_{A} receptors. In some embodiments, neuroactive steroids act as modulators of γ-aminobutyric acid (GABA) receptor complex (GRC) in the central nervous system (CNS). Examples include, but are not limited to, tetrahydrodeoxycorticosterone (THDOC), androstane, androstane 3α-androstanediol, cholestane cholesterol, pregnane, pregnane pregnanolone (eltanolone), allopregnanolone, brexanolone, ganaxolone and SAGE-217.

As used herein, the term “pharmaceutically acceptable salts” includes those obtained by reacting the active compound functioning as a base, with an inorganic or organic acid to form a salt, for example, salts of 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, maleic acid, malic acid, (−L) malonic acid, mandelic acid (DL), methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, propionic acid, pyroglutamic acid (−L), salicylic acid, sebacic acid, stearic acid, succinic acid, sulfuric acid, tartaric acid (+L), thiocyanic acid, toluenesulfonic acid (p), and undecylenic acid. Those skilled in the art will further recognize that acid addition salts may be prepared by reaction of the compounds with the appropriate inorganic or organic acid via any of a number of known methods.

As used herein, the term “derivative” or grammatical variations thereof, either in singular or in plural form, can refer to a compound that is derived from a similar compound by a chemical reaction. For example, a derivative of brexanolone can be derived from brexanolone by a chemical reaction.

As used herein, the term “particles”, “particle” or grammatical variations thereof, either in singular or in plural form, can refer to particles disclosed herein and, in some examples, can also refer to stabilized particles that are stable under physiological conditions without changing its physical or chemical form for an extended period of time, such as for a time period in a range of from 0.1 to 20 hours, 1 to 50 hours, 2 to 75 hours, 5 to 100 hours, 1 to 5 days, 2 to 7 days, 3 to 10 days, 4 to 20 days, or longer.

As used herein, the term “particle size” refers to a primary particle size or crystallite size that is the smallest particle size. When particles of primary size aggregate together, the aggregate can have an aggregate particle size that is typically a multiple of the primary particle size. The particle size used herein refers to the largest dimension of a primary particle, for example, a diameter of a spherical particle, a longest length of a rod or bar shaped particle, or a largest size measured across an irregular shaped particle.

As used herein, the term “mean particle size” refers to an average of particle sizes of the particles measured or selected. In one example, mean particle size can be calculated by dividing the sum of the particle sizes by the number of particles measured or selected.

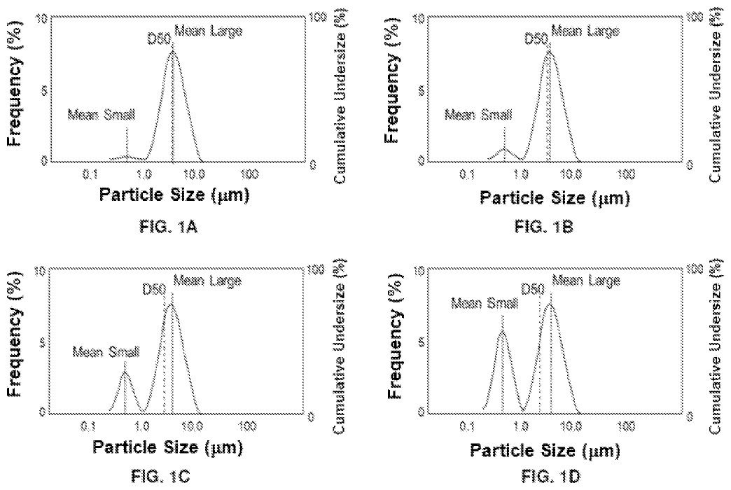

As used herein, the terms D10 (or Dv10), D50 (or Dv50) and D90 (or Dv90) are commonly used to represent the midpoint and range of the particle sizes of a given sample. The term “D10” refers to 10% of particles are below and 90% of particles are above a defined measurement, for example a particle diameter. The term “D50” refers to a mass-median-diameter (MMD), wherein 50% of particles are below and 50% of particles are above a defined measurement, for example a particle diameter. The term “D90” refers to 90% of particles are below and 10% of particles are above a defined measurement, for example a particle diameter. In some embodiments, D50=1.5 μm means 50% of particles are below 1.5 μm and 50% of particle are above 1.5 μm. In some embodiments, D90=4.0 μm means 90% particles are below 4.0 μm in diameter and 10% of particles are above 4.0 μm in diameter. The percentage can be based on total volume of particles, total weight of particles, total number or counts of particles measured, or a total area of the particles measured. In some embodiments, a sample of particles are measured by light scattering with about 1×10⁶ particles measured. A measurement data of D50=0.9 μm means about 50% of particles measured are below 0.9 μm and 50% are above 0.9 μm, percentage based on the total number of particles measured. In some embodiments, particle sizes are measured using microscopy and imaging technology or optical granulometry techniques, wherein particles in certain fields are measured. With this, a percentage can be based on the total number of particles measured or a given area measured. For example, the phrase “10% of the particles having a median particle size of about 0.2-15 μm” refers to 10% of the particles measured from one or more samples of those particles having a median particle size of about 0.2-15 μm based on the total number of particles measured. The phrase “10% of the particles having a mean particle size of about 0.2-15 μm” refers to 10% of the particles measured from one or more samples of those particles having a mean particle size of about 0.2-15 μm based on the total number of particles measured. Unless specified otherwise, a percentage of particles having a certain size or a range of sizes refers to the percentage based on the total number of particles measured from one or more samples of those particles. Non-limiting examples are shown in FIG. 1A-FIG. 1D.

As used herein, the term “Cₘₐₓ”, “Cmax” or “maximum plasma concentration” refers to the maximum (or peak) plasma concentration that a drug reaches in a specified compartment or part of the body after the drug has been administered and before the administration of a second dose. In some cases, the term “C_{max, 1}” and “C_{max, 2}” refer to the first and second peak plasma concentrations of the drug after administration, respectively. In some cases, the Cₘₐₓ is measured from specimens from a subject, such blood samples or serum sample from the subject.

As used herein, the term “extended-release” or “ER” refers to a mechanism that (in contrast to immediate-release) delivers a drug for a prolonged period of time; it is meant to include any dosage form or formulation which is not an immediate release dosage form or formulation including those described in Chapter 17 of “Applied Biopharmaceutics and Pharmacokinetics”, Sixth Edition; Shargel et al., which is incorporated herein by reference. In some cases, an extended-release dosage form can maintain a plasma concentration in a subject at a level more than about 5%, 10%, 15%, 20%, 25%, 30%, 35%, 40%, 45%, 50%, 55%, 60%, 65%, or 70% of the Cmax for more than 1, 2, 3, 4, 5, 6, 7, 8, 9, 10, 11, 12, 13, or 14 days after administering to the subject as disclosed hereafter. For the purposes herein, ER dosage forms also includes “sustained release” or “SR” dosage forms.

As used herein, “an “effective amount” refers to a therapeutically effective amount or a prophylactically effective amount. A “therapeutically effective amount” refers to an amount effective, at dosages and for periods of time necessary, to achieve the desired therapeutic result. In some cases, a therapeutically effective amount refers to an amount that provides a therapeutically effective plasma concentration and/or exposure of a drug substance. A therapeutically effective amount of a compound can vary according to factors such as the disease state, age, sex, and weight of the subject, and the ability of the compound to elicit a desired response in the subject. Dosage regimens can be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as smaller tumors, increased life span, increased life expectancy or prevention of the progression of prostate cancer to a castration-resistant form. Typically, a prophylactic dose is used in subjects prior to or at an earlier stage of disease, so that a prophylactically effective amount can be less than a therapeutically effective amount.

As used herein, “treating” or “treatment” covers the treatment of the disease or condition of interest in a mammal, for example in a human, having the disease or condition of interest, and includes (but is not limited to): 1) preventing the disease or condition from occurring in a mammal, in particular, when such mammal is predisposed to the condition but has not yet been diagnosed as having it; 2) inhibiting the disease or condition, i.e., arresting its development; 3) relieving the disease or condition, i.e., causing regression of the disease or condition (ranging from reducing the severity of the disease or condition to curing the disease of condition); or 4) relieving the symptoms resulting from the disease or condition, i.e., relieving pain without addressing the underlying disease or condition.

As used herein, the terms “preventing”, “prevention” and “prophylactic treatment” refer to a reduction in risk of acquiring or developing a disease or disorder (i.e., causing at least one of the clinical symptoms of the disease not to develop in a subject not yet exposed to a disease-causing agent, or predisposed to the disease in advance of disease onset). The term “prophylaxis” is related to “prevention,” and refers to a measure or procedure the purpose of which is to prevent, rather than to treat or cure a disease.

As used herein, the terms “disease” and “condition” can be used interchangeably or can be different in that the particular malady or condition cannot have a known causative agent (so that etiology has not yet been worked out) and it is therefore not yet recognized as a disease, but only as an undesirable condition or syndrome, wherein a more or less specific set of symptoms have been identified by clinicians.

As used herein, a “subject” can be a human, non-human primate, mammal, rat, mouse, cow, horse, pig, sheep, goat, dog, cat, insect and the like. The subject can be suspected of having or at risk for having a neurological condition, such as postpartum depression (PPD). Particularly in the case of PPD, the subject can be a lactating woman who is breastfeeding an infant frequently or on a regular basis. The term “breastfeeding” or a grammatical variation refers to delivering breast milk of the woman directly to an infant, extracting breast milk from the woman using a device and subsequently delivering to the infant, extracting breast milk from the woman using a device and storing the breast milk for a short period of time and subsequently delivering the stored breast milk to the infant, or a combination thereof.

As used herein, the term “family history” of a subject means a record of diseases and health conditions in the subject's family including parents, sisters, brothers, half-sisters, half-brothers, children, grandparents, aunts, uncles, nieces, and nephews.

As used herein, recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

The use of numerical values in the various ranges specified in this application, unless expressly indicated otherwise, are stated as approximations as though the minimum and maximum values within the stated ranges were both proceeded by the word “about.” In this manner, slight variations above and below the stated ranges can be used to achieve substantially the same results as values within the ranges. Also, the disclosure of these ranges is intended as a continuous range including every value between the minimum and maximum values.

As used herein, the term “about” and its grammatical equivalents in relation to a reference numerical value and its grammatical equivalents as used herein can include a range of values plus or minus 10% from that value, such as a range of values plus or minus 10%, 9%, 8%, 7%, 6%, 5%, 4%, 3%, 2%, or 1% from that value. For example, the amount “about 10” includes amounts from 9 to 11.

### DETAILED DESCRIPTION

Disclosed herein is a novel pharmaceutical composition comprising a neuroactive steroid and its use thereof. In some cases, the neuroactive steroid can be brexanolone. For example, an aqueous suspension pharmaceutical composition of brexanolone can be used as one or more injections for the treatment of PPD and other neurological conditions and/or can replace the prolonged IV infusion of ZULRESSO™. The aqueous suspension pharmaceutical composition can be an extended-release brexanolone for intramuscular injection, which provides a duration of minimum effective plasma exposure of about at least about 20, 24, 28, 32, 36, 40, 44, 48, 52, 56, 60, 64, 68, 72, 96, 120, 144, 168, 192, 216, 240, or 336 hours. In some cases, the extended-release brexanolone provides a duration of minimum effective plasma exposure of 28-48 h, which is consistent with the brexanolone IV infusion protocol. In some cases, the extended-release brexanolone from IM depot into systemic circulation can lead to a gradual and slow taper, and therefore a relatively longer duration of plasma exposure (compared to brexanolone IV) driven by a longer apparent elimination half-life. Among the benefits over the current treatment can be ease of administration, an improved safety profile, and/or patient convenience. In some aspects, the pharmaceutical composition and method disclosed herein can provide one or more of the following advantages.

First, ease of administration. The method can administer a single dosage via intramuscular injection in a few seconds eliminating the complicated IV infusion for many hours, such as 6 to 60 hours in some current treatment. Unlike those IV infusions, such as those of ZULRESSO™, single injection extended-release brexanolone can minimize the risk of overdose and, by design, providing a predictable exposure pattern that avoids unexpectedly high levels or sudden changes of brexanolone in circulation. This profile should reduce risk of GABAergic withdrawal adverse events and can potentially improve longer-term efficacy.

Second, patient convenience. One or more injections can be administered by a healthcare provider as an outpatient instead of an inpatient (or infusion center) for 60-hour infusion requiring direct clinical observation. Extended-release brexanolone can allow patients to have fewer injections and longer intervals between injections. The single injection of the extended-release brexanolone can also be beneficial to many women for improved patient adherence and compliance that can reduce the risk of developing PPD in women due to cognitive impairment and apathy which may reduce adherence.

Third, better healthcare resource utilization. Single dosage injection, such as IM or SC injections, of the extended-release dosage form can reduce the requirement of costly infusion centers and inpatient care, the needs for IV placement and infusion equipment, and requirement for continual observation by a healthcare provider.

Fourth, better infant safety. The extended-release dosage form would be administered immediately after birth, thereby avoiding systemic exposure to the fetus. Additionally, exposure to the infant via breastmilk is negligible due to the very low oral bioavailability of brexanolone.

### Drug Product

The present disclosure relates to extended-release injectable formulations of the neuroactive steroid (NAS) gamma-aminobutyric acid (GABA_{A}) receptor positive allosteric modulator (PAM), brexanolone (synthetic allopregnanolone). Brexanolone, known also by its chemical IUPAC name 1-(3R,5S,8R,9S,10S,13S,14S,17S)-3-hydroxy-10,13-dimethylhexadecahydro-1H-cyclopenta[a]phenanthren-17-yl) ethan-1-one or as synthetic allopregnanolone, has the chemical structure of Compound 1:

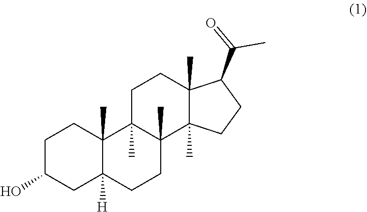

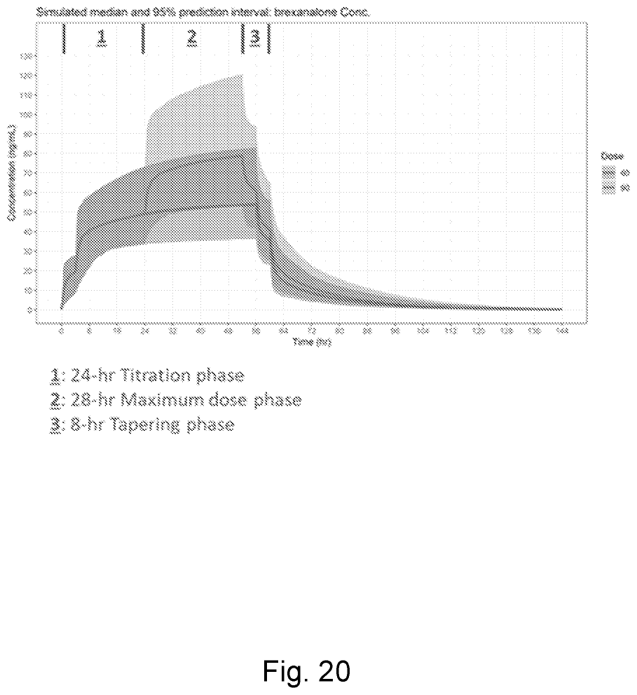

Brexanolone is currently marketed as ZULRESSO™, a continuous intravenous infusion that is administered over 60 hours. ZULRESSO™ has been approved by the FDA for the treatment of postpartum depression (PPD), but drug administration requires a complex infusion protocol and it carries a warning for excessive sedation and sudden loss of consciousness. Thus, ZULRESSO™ is available only through a restricted access program and patients must be carefully monitored. FIG. 20 shows the simulated plasma concentration—time curves for a 60-hour IV infusion of Zulresso™ in human subjects (US Food and Drug Administration, Center for Drug Evaluation and Research 2018. Multi-disciplinary review and evaluation, NDA 211371, Zulresso™ [brexanolone]).

### Pharmaceutical Compositions and Formulations

Disclosed herein are aqueous suspension pharmaceutical compositions comprising a pharmaceutically effective amount of a neuroactive steroid, or a pharmaceutically acceptable salt or derivative thereof. In some embodiments, the neuroactive steroid provides a therapeutically effective plasma concentration over a period of at least about 72 hours to treat a neurological condition when administered in one or more injections to a subject in need thereof.

In some embodiments, the neuroactive steroid comprises one or more positive allosteric modulators of the γ aminobutyric acid type A receptors (GABA_{A}) selected from tetrahydrodeoxycorticosterone (THDOC), androstane, androstane 3α-androstanediol, cholestane cholesterol, pregnane, eltanolone, brexanolone, ganaxolone, zuranolone, or a combination thereof. In some embodiments, the neuroactive steroid is selected from the group consisting of brexanolone, pharmaceutically acceptable salts, and derivatives thereof.

Advantageously, in some embodiments, the present pharmaceutical compositions achieve equivalent therapeutic efficacy by providing controlled and slow release of brexanolone from IM depot into systemic circulation. This leads to a gradual and slow taper, and consequently, a relatively longer duration of plasma exposure (compared to brexanolone IV) driven by a longer apparent terminal elimination half-life. Without being bound by any particular theory, a longer terminal elimination may avoid a rapid drop-off of physiological allopregnanolone levels, which is hypothesized to be, in part, responsible for initiation of PPD.

### Neuroactive Steroid

In some embodiments, the aqueous suspension pharmaceutical composition comprises a neuroactive steroid (e.g., brexanolone) in an amount ranging from about 10 mg to about 1,500 mg. In some embodiments, the amount of the neuroactive steroid (e.g., brexanolone) is at least about 10 mg. In some embodiments, the amount of the neuroactive steroid (e.g., brexanolone) is at most about 1,500 mg. In some embodiments, the amount of the neuroactive steroid (e.g., brexanolone) is about 10 mg to about 30 mg, about 10 mg to about 50 mg, about 10 mg to about 100 mg, about 10 mg to about 200 mg, about 10 mg to about 300 mg, about 10 mg to about 400 mg, about 10 mg to about 500 mg, about 10 mg to about 600 mg, about 10 mg to about 800 mg, about 10 mg to about 1,000 mg, about 10 mg to about 1,500 mg, about 30 mg to about 50 mg, about 30 mg to about 100 mg, about 30 mg to about 200 mg, about 30 mg to about 300 mg, about 30 mg to about 400 mg, about 30 mg to about 500 mg, about 30 mg to about 600 mg, about 30 mg to about 800 mg, about 30 mg to about 1,000 mg, about 30 mg to about 1,500 mg, about 50 mg to about 100 mg, about 50 mg to about 200 mg, about 50 mg to about 300 mg, about 50 mg to about 400 mg, about 50 mg to about 500 mg, about 50 mg to about 600 mg, about 50 mg to about 800 mg, about 50 mg to about 1,000 mg, about 50 mg to about 1,500 mg, about 100 mg to about 200 mg, about 100 mg to about 300 mg, about 100 mg to about 400 mg, about 100 mg to about 500 mg, about 100 mg to about 600 mg, about 100 mg to about 800 mg, about 100 mg to about 1,000 mg, about 100 mg to about 1,500 mg, about 200 mg to about 300 mg, about 200 mg to about 400 mg, about 200 mg to about 500 mg, about 200 mg to about 600 mg, about 200 mg to about 800 mg, about 200 mg to about 1,000 mg, about 200 mg to about 1,500 mg, about 300 mg to about 400 mg, about 300 mg to about 500 mg, about 300 mg to about 600 mg, about 300 mg to about 800 mg, about 300 mg to about 1,000 mg, about 300 mg to about 1,500 mg, about 400 mg to about 500 mg, about 400 mg to about 600 mg, about 400 mg to about 800 mg, about 400 mg to about 1,000 mg, about 400 mg to about 1,500 mg, about 500 mg to about 600 mg, about 500 mg to about 800 mg, about 500 mg to about 1,000 mg, about 500 mg to about 1,500 mg, about 600 mg to about 800 mg, about 600 mg to about 1,000 mg, about 600 mg to about 1,500 mg, about 800 mg to about 1,000 mg, about 800 mg to about 1,500 mg, or about 1,000 mg to about 1,500 mg. In some embodiments, the amount of the neuroactive steroid (e.g., brexanolone) is about 10 mg, about 30 mg, about 50 mg, about 100 mg, about 200 mg, about 300 mg, about 400 mg, about 500 mg, about 600 mg, about 800 mg, about 1,000 mg, or about 1,500 mg, including all ranges and values therebetween.

In some embodiments, the aqueous suspension pharmaceutical composition comprises a neuroactive steroid (e.g., brexanolone) in a concentration ranging from about 5 mg/mL to about 800 mg/mL. In some embodiments, the concentration of the neuroactive steroid (e.g., brexanolone) is at least about 5 mg/mL. In some embodiments, the concentration of the neuroactive steroid (e.g., brexanolone) is at most about 800 mg/mL. In some embodiments, the concentration of the neuroactive steroid (e.g., brexanolone) is about 5 mg/mL to about 10 mg/mL, about 5 mg/mL to about 30 mg/mL, about 5 mg/mL to about 50 mg/mL, about 5 mg/mL to about 100 mg/mL, about 5 mg/mL to about 150 mg/mL, about 5 mg/mL to about 200 mg/mL, about 5 mg/mL to about 250 mg/mL, about 5 mg/mL to about 300 mg/mL, about 5 mg/mL to about 400 mg/mL, about 5 mg/mL to about 500 mg/mL, about 5 mg/mL to about 800 mg/mL, about 10 mg/mL to about 30 mg/mL, about 10 mg/mL to about 50 mg/mL, about 10 mg/mL to about 100 mg/mL, about 10 mg/mL to about 150 mg/mL, about 10 mg/mL to about 200 mg/mL, about 10 mg/mL to about 250 mg/mL, about 10 mg/mL to about 300 mg/mL, about 10 mg/mL to about 400 mg/mL, about 10 mg/mL to about 500 mg/mL, about 10 mg/mL to about 800 mg/mL, about 30 mg/mL to about 50 mg/mL, about 30 mg/mL to about 100 mg/mL, about 30 mg/mL to about 150 mg/mL, about 30 mg/mL to about 200 mg/mL, about 30 mg/mL to about 250 mg/mL, about 30 mg/mL to about 300 mg/mL, about 30 mg/mL to about 400 mg/mL, about 30 mg/mL to about 500 mg/mL, about 30 mg/mL to about 800 mg/mL, about 50 mg/mL to about 100 mg/mL, about 50 mg/mL to about 150 mg/mL, about 50 mg/mL to about 200 mg/mL, about 50 mg/mL to about 250 mg/mL, about 50 mg/mL to about 300 mg/mL, about 50 mg/mL to about 400 mg/mL, about 50 mg/mL to about 500 mg/mL, about 50 mg/mL to about 800 mg/mL, about 100 mg/mL to about 150 mg/mL, about 100 mg/mL to about 200 mg/mL, about 100 mg/mL to about 250 mg/mL, about 100 mg/mL to about 300 mg/mL, about 100 mg/mL to about 400 mg/mL, about 100 mg/mL to about 500 mg/mL, about 100 mg/mL to about 800 mg/mL, about 150 mg/mL to about 200 mg/mL, about 150 mg/mL to about 250 mg/mL, about 150 mg/mL to about 300 mg/mL, about 150 mg/mL to about 400 mg/mL, about 150 mg/mL to about 500 mg/mL, about 150 mg/mL to about 800 mg/mL, about 200 mg/mL to about 250 mg/mL, about 200 mg/mL to about 300 mg/mL, about 200 mg/mL to about 400 mg/mL, about 200 mg/mL to about 500 mg/mL, about 200 mg/mL to about 800 mg/mL, about 250 mg/mL to about 300 mg/mL, about 250 mg/mL to about 400 mg/mL, about 250 mg/mL to about 500 mg/mL, about 250 mg/mL to about 800 mg/mL, about 300 mg/mL to about 400 mg/mL, about 300 mg/mL to about 500 mg/mL, about 300 mg/mL to about 800 mg/mL, about 400 mg/mL to about 500 mg/mL, about 400 mg/mL to about 800 mg/mL, or about 500 mg/mL to about 800 mg/mL. In some embodiments, the concentration of the neuroactive steroid (e.g., brexanolone) is about 5 mg/mL, about 10 mg/mL, about 30 mg/mL, about 50 mg/mL, about 100 mg/mL, about 150 mg/mL, about 200 mg/mL, about 250 mg/mL, about 300 mg/mL, about 400 mg/mL, about 500 mg/mL, or about 800 mg/mL, including all ranges and values therebetween.

In some embodiments, the amount of neuroactive steroid (e.g., brexanolone) is from about 0.01% to about 99% (w/w) of the aqueous suspension pharmaceutical composition, for example, about 0.1%-1%, about 0.1%-5%, about 0.1-10%, about 0.1%-20%, about 0.5%-1%, about 0.5%-5%, about 0.5%-10%, about 0.5%-20%, about 1%-5%, about 1%-10%, about 1%-20%, about 5%-10%, about 5%-20%, about 10%-20%, about 10%-30%, about 20%-30%, about 20%-40%, about 30%-40%, about 30%-50%, about 40%-50%, about 40%-60%, about 50%-60%, about 50%-70%, about 60%-70%, about 60%-80%, about 70%-80%, about 70%-90%, about 80%-90%, about 80%-95%, or 95%-99% of the pharmaceutical composition. In some embodiments, the amount of neuroactive steroid is from about 0.1% to about 70%, or from about 1% to about 30% of the pharmaceutical composition.

In some embodiments, a single dose of the neuroactive steroid (e.g., brexanolone) in the disclosed pharmaceutical composition can be about 0.5 mg to about 50 mg per kilogram (kg) of body weight. In some embodiments, a single dose can be at least about 0.5 mg per kg of body weight. In some embodiments, a single dose can be at most about 50 mg per kg of body weight. In some embodiments, a single dose can be about 0.5 mg to about 2 mg per kg of body weight, about 0.5 mg to about 4 mg per kg of body weight, about 0.5 mg to about 6 mg per kg of body weight, about 0.5 mg to about 8 mg per kg of body weight, about 0.5 mg to about 10 mg per kg of body weight, about 0.5 mg to about 20 mg per kg of body weight, about 0.5 mg to about 50 mg per kg of body weight, about 2 mg to about 4 mg per kg of body weight, about 2 mg to about 6 mg per kg of body weight, about 2 mg to about 8 mg per kg of body weight, about 2 mg to about 10 mg per kg of body weight, about 2 mg to about 20 mg per kg of body weight, about 2 mg to about 50 mg per kg of body weight, about 4 mg to about 6 mg per kg of body weight, about 4 mg to about 8 mg per kg of body weight, about 4 mg to about 10 mg per kg of body weight, about 4 mg to about 20 mg per kg of body weight, about 4 mg to about 50 mg per kg of body weight, about 6 mg to about 8 mg per kg of body weight, about 6 mg to about 10 mg per kg of body weight, about 6 mg to about 20 mg per kg of body weight, about 6 mg to about 50 mg per kg of body weight, about 8 mg to about 10 mg per kg of body weight, about 8 mg to about 20 mg per kg of body weight, about 8 mg to about 50 mg per kg of body weight, about 10 mg to about 20 mg per kg of body weight, about 10 mg to about 50 mg per kg of body weight, or about 20 mg to about 50 mg per kg of body weight. In some embodiments, a single dose can be about 0.5 mg per kg of body weight, about 2 mg per kg of body weight, about 4 mg per kg of body weight, about 6 mg per kg of body weight, about 8 mg per kg of body weight, about 10 mg per kg of body weight, about 20 mg per kg of body weight, or about 50 mg per kg of body weight. In a particular example, the single dose can be about 3.5 mg to 5 mg per kg of body weight. The body weight refers to the body weight of a subject, such as a human patient or an animal subject.

In some embodiments, a unit dose the neuroactive steroid (e.g., brexanolone) in the disclosed pharmaceutical composition can be about 50 mg to about 800 mg. In some embodiments, a single unit dose can be at least about 50 mg. In some embodiments, a single unit dose can be at most about 800 mg. In some embodiments, a single unit dose can be about 50 mg to about 100 mg, about 50 mg to about 200 mg, about 50 mg to about 300 mg, about 50 mg to about 400 mg, about 50 mg to about 600 mg, about 50 mg to about 800 mg, about 100 mg to about 200 mg, about 100 mg to about 300 mg, about 100 mg to about 400 mg, about 100 mg to about 600 mg, about 100 mg to about 800 mg, about 200 mg to about 300 mg, about 200 mg to about 400 mg, about 200 mg to about 600 mg, about 200 mg to about 800 mg, about 300 mg to about 400 mg, about 300 mg to about 600 mg, about 300 mg to about 800 mg, about 400 mg to about 600 mg, about 400 mg to about 800 mg, or about 600 mg to about 800 mg. In some embodiments, a single unit dose can be about 50 mg, about 100 mg, about 200 mg, about 300 mg, about 400 mg, about 600 mg, or about 800 mg. A unit dose is a form of package of the pharmaceutical composition that can be administered to a subject in a single dose. For example, a 300 mg unit dose of a pharmaceutical composition can be packaged in a certain volume, such as one milliliter volume, in an injectable form that can be injected into a subject in one or more injections. In yet other examples, a 300 mg unit dose of a pharmaceutical composition can be packaged in a certain volume, such as 0.5 milliliter volume or one milliliter volume, in an injectable form that can be injected into a subject in a single subcutaneous injection.

In some embodiments, a single dose of the neuroactive steroid (e.g., brexanolone) in the disclosed pharmaceutical composition can be about 50 mg to about 800 mg. In some embodiments, a single dose can be at least about 50 mg. In some embodiments, a single dose can be at most about 800 mg. In some embodiments, a single dose can be about 50 mg to about 100 mg, about 50 mg to about 200 mg, about 50 mg to about 300 mg, about 50 mg to about 400 mg, about 50 mg to about 600 mg, about 50 mg to about 800 mg, about 100 mg to about 200 mg, about 100 mg to about 300 mg, about 100 mg to about 400 mg, about 100 mg to about 600 mg, about 100 mg to about 800 mg, about 200 mg to about 300 mg, about 200 mg to about 400 mg, about 200 mg to about 600 mg, about 200 mg to about 800 mg, about 300 mg to about 400 mg, about 300 mg to about 600 mg, about 300 mg to about 800 mg, about 400 mg to about 600 mg, about 400 mg to about 800 mg, or about 600 mg to about 800 mg. In some embodiments, a single dose can be about 50 mg, about 100 mg, about 200 mg, about 300 mg, about 400 mg, about 600 mg, or about 800 mg.

A single dose can be adjusted when using a unit dose to administer the pharmaceutical composition to a subject based on the body weight of the subject. In one example, a unit dose of 300 mg in 1 mL injectable solution is designed for a single dose injection to a subject of body weight in a range of from 60 kg to 70 kg. For a subject having body weight less than 60 kg, an adjusted dose, such as 0.5 mL of the 300 mg unit dose, can be injected to the subject in one injection. For a subject having body weight more than 70 kg, an adjusted dose, such as 1.5 mL of the 300 mg unit dose, can be injected to the subject in one injection. The single dose can be adjusted to have the required mg of the pharmaceutical composition per kilogram (kg) of body weight as disclosed herein.

The single dose of the disclosed pharmaceutical composition can be in a range of from about 0.5 to 50 mg per kilogram (kg) of body weight and can be produced by combining one or more unit doses, or a part thereof, wherein each of the unit doses can be in a range of from 50 mg to 800 mg per unit dose. The ranges of single dose, unit dose or a combination thereof disclosed above and hereafter are suitable and are incorporated as examples.

In some embodiments, the neuroactive steroid (e.g., brexanolone) has a particle size distribution (PSD) with a D10 ranging from about 0.5 μm to about 3 μm. In some embodiments, the D10 of neuroactive steroid (e.g., brexanolone) is at least about 0.5 μm. In some embodiments, the D10 of neuroactive steroid (e.g., brexanolone) is at most about 3 μm. In some embodiments, the D10 of neuroactive steroid (e.g., brexanolone) is about 0.5 μm to about 1 μm, about 0.5 μm to about 1.2 μm, about 0.5 μm to about 1.4 μm, about 0.5 μm to about 1.6 μm, about 0.5 μm to about 1.8 μm, about 0.5 μm to about 2 μm, about 0.5 μm to about 2.2 μm, about 0.5 μm to about 2.4 μm, about 0.5 μm to about 2.6 μm, about 0.5 μm to about 2.8 μm, about 0.5 μm to about 3 μm, about 1 μm to about 1.2 μm, about 1 μm to about 1.4 μm, about 1 μm to about 1.6 μm, about 1 μm to about 1.8 μm, about 1 μm to about 2 μm, about 1 μm to about 2.2 μm, about 1 μm to about 2.4 μm, about 1 μm to about 2.6 μm, about 1 μm to about 2.8 μm, about 1 μm to about 3 μm, about 1.2 μm to about 1.4 μm, about 1.2 μm to about 1.6 μm, about 1.2 μm to about 1.8 μm, about 1.2 μm to about 2 μm, about 1.2 μm to about 2.2 μm, about 1.2 μm to about 2.4 μm, about 1.2 μm to about 2.6 μm, about 1.2 μm to about 2.8 μm, about 1.2 μm to about 3 μm, about 1.4 μm to about 1.6 μm, about 1.4 μm to about 1.8 μm, about 1.4 μm to about 2 μm, about 1.4 μm to about 2.2 μm, about 1.4 μm to about 2.4 μm, about 1.4 μm to about 2.6 μm, about 1.4 μm to about 2.8 μm, about 1.4 μm to about 3 μm, about 1.6 μm to about 1.8 μm, about 1.6 μm to about 2 μm, about 1.6 μm to about 2.2 μm, about 1.6 μm to about 2.4 μm, about 1.6 μm to about 2.6 μm, about 1.6 μm to about 2.8 μm, about 1.6 μm to about 3 μm, about 1.8 μm to about 2 μm, about 1.8 μm to about 2.2 μm, about 1.8 μm to about 2.4 μm, about 1.8 μm to about 2.6 μm, about 1.8 μm to about 2.8 μm, about 1.8 μm to about 3 μm, about 2 μm to about 2.2 μm, about 2 μm to about 2.4 μm, about 2 μm to about 2.6 μm, about 2 μm to about 2.8 μm, about 2 μm to about 3 μm, about 2.2 μm to about 2.4 μm, about 2.2 μm to about 2.6 μm, about 2.2 μm to about 2.8 μm, about 2.2 μm to about 3 μm, about 2.4 μm to about 2.6 μm, about 2.4 μm to about 2.8 μm, about 2.4 μm to about 3 μm, about 2.6 μm to about 2.8 μm, about 2.6 μm to about 3 μm, or about 2.8 μm to about 3 μm. In some embodiments, the D10 of neuroactive steroid (e.g., brexanolone) is about 0.5 μm, about 1 μm, about 1.2 μm, about 1.4 μm, about 1.6 μm, about 1.8 μm, about 2 μm, about 2.2 μm, about 2.4 μm, about 2.6 μm, about 2.8 μm, or about 3 μm, including all ranges and values therebetween.

In some embodiments, the neuroactive steroid (e.g., brexanolone) has a particle size distribution (PSD) with a D50 ranging from about 1 μm to about 10 μm. In some embodiments, the D50 of neuroactive steroid (e.g., brexanolone) is at least about 1 μm. In some embodiments, the D50 of neuroactive steroid (e.g., brexanolone) is at most about 10 μm. In some embodiments, the D50 of neuroactive steroid (e.g., brexanolone) is about 1 μm to about 2 μm, about 1 μm to about 3 μm, about 1 μm to about 4 μm, about 1 μm to about 5 μm, about 1 μm to about 6 μm, about 1 μm to about 7 μm, about 1 μm to about 8 μm, about 1 μm to about 9 μm, about 1 μm to about 10 μm, about 2 μm to about 3 μm, about 2 μm to about 4 μm, about 2 μm to about 5 μm, about 2 μm to about 6 μm, about 2 μm to about 7 μm, about 2 μm to about 8 μm, about 2 μm to about 9 μm, about 2 μm to about 10 μm, about 3 μm to about 4 μm, about 3 μm to about 5 μm, about 3 μm to about 6 μm, about 3 μm to about 7 μm, about 3 μm to about 8 μm, about 3 μm to about 9 μm, about 3 μm to about 10 μm, about 4 μm to about 5 μm, about 4 μm to about 6 μm, about 4 μm to about 7 μm, about 4 μm to about 8 μm, about 4 μm to about 9 μm, about 4 μm to about 10 μm, about 5 μm to about 6 μm, about 5 μm to about 7 μm, about 5 μm to about 8 μm, about 5 μm to about 9 μm, about 5 μm to about 10 μm, about 6 μm to about 7 μm, about 6 μm to about 8 μm, about 6 μm to about 9 μm, about 6 μm to about 10 μm, about 7 μm to about 8 μm, about 7 μm to about 9 μm, about 7 μm to about 10 μm, about 8 μm to about 9 μm, about 8 μm to about 10 μm, or about 9 μm to about 10 μm. In some embodiments, the D50 of neuroactive steroid (e.g., brexanolone) is about 1 μm, about 2 μm, about 3 μm, about 4 μm, about 5 μm, about 6 μm, about 7 μm, about 8 μm, about 9 μm, or about 10 μm, including all ranges and values therebetween. In some embodiments, the D50 of brexanolone is about 3 μm, e.g., about 3 μm, 3.05 μm, 3.1 μm, 3.2 μm, 3.3 μm, or 3.4 μm, including all values therebetween.

In some embodiments, the neuroactive steroid (e.g., brexanolone) has a particle size distribution (PSD) with a D90 ranging from about 4 μm to about 15 μm. In some embodiments, the D90 of neuroactive steroid (e.g., brexanolone) is at least about 4 μm. In some embodiments, the D90 of neuroactive steroid (e.g., brexanolone) is at most about 15 μm. In some embodiments, the D90 of neuroactive steroid (e.g., brexanolone) is about 4 μm to about 5 μm, about 4 μm to about 6 μm, about 4 μm to about 7 μm, about 4 μm to about 8 μm, about 4 μm to about 9 μm, about 4 μm to about 10 μm, about 4 μm to about 11 μm, about 4 μm to about 12 μm, about 4 μm to about 13 μm, about 4 μm to about 14 μm, about 4 μm to about 15 μm, about 5 μm to about 6 μm, about 5 μm to about 7 μm, about 5 μm to about 8 μm, about 5 μm to about 9 μm, about 5 μm to about 10 μm, about 5 μm to about 11 μm, about 5 μm to about 12 μm, about 5 μm to about 13 μm, about 5 μm to about 14 μm, about 5 μm to about 15 μm, about 6 μm to about 7 μm, about 6 μm to about 8 μm, about 6 μm to about 9 μm, about 6 μm to about 10 μm, about 6 μm to about 11 μm, about 6 μm to about 12 μm, about 6 μm to about 13 μm, about 6 μm to about 14 μm, about 6 μm to about 15 μm, about 7 μm to about 8 μm, about 7 μm to about 9 μm, about 7 μm to about 10 μm, about 7 μm to about 11 μm, about 7 μm to about 12 μm, about 7 μm to about 13 μm, about 7 μm to about 14 μm, about 7 μm to about 15 μm, about 8 μm to about 9 μm, about 8 μm to about 10 μm, about 8 μm to about 11 μm, about 8 μm to about 12 μm, about 8 μm to about 13 μm, about 8 μm to about 14 μm, about 8 μm to about 15 μm, about 9 μm to about 10 μm, about 9 μm to about 11 μm, about 9 μm to about 12 μm, about 9 μm to about 13 μm, about 9 μm to about 14 μm, about 9 μm to about 15 μm, about 10 μm to about 11 μm, about 10 μm to about 12 μm, about 10 μm to about 13 μm, about 10 μm to about 14 μm, about 10 μm to about 15 μm, about 11 μm to about 12 μm, about 11 μm to about 13 μm, about 11 μm to about 14 μm, about 11 μm to about 15 μm, about 12 μm to about 13 μm, about 12 μm to about 14 μm, about 12 μm to about 15 μm, about 13 μm to about 14 μm, about 13 μm to about 15 μm, or about 14 μm to about 15 μm. In some embodiments, the D90 of neuroactive steroid (e.g., brexanolone) is about 4 μm, about 5 μm, about 6 μm, about 7 μm, about 8 μm, about 9 μm, about 10 μm, about 11 μm, about 12 μm, about 13 μm, about 14 μm, or about 15 μm, including all ranges and values therebetween. In some embodiments, the D90 of brexanolone is about 6 μm, e.g., about 6 μm, 6.05 μm, 6.1 μm, 6.2 μm, 6.3 μm, or 6.4 μm, including all values therebetween.

In some embodiments, the particle size distribution of the neuroactive steroid (e.g., brexanolone) is unimodal. In some embodiments, the particle size of the neuroactive steroid (e.g., brexanolone) has no more than 5%, 10%, 15%, 20%, 25%, or 30% standard deviation from the mean (D50) particle size. In some embodiments, the particle size of the neuroactive steroid (e.g., brexanolone) has no more than 10% standard deviation from the mean (D50) particle size. In some embodiments, the particle size of the neuroactive steroid (e.g., brexanolone) has no more than 20% standard deviation from the mean (D50) particle size. In some embodiments, the particle size of the neuroactive steroid (e.g., brexanolone) has no more than 30% standard deviation from the mean (D50) particle size.

A pharmaceutical composition disclosed herein comprising a neurosteroid (e.g., brexanolone) can comprise about 0.01% to about 50% of small particles and about 50% to about 99.99% of large particles, percentage based on the total counts of the particles measured. Such pharmaceutical composition comprising a neurosteroid can comprise particles in a range of from 0.01% to 50% in one embodiment, 0.1% to 50% in another embodiment, 1.0% to 50% in yet another embodiment, 2.0% to 50% in yet another embodiment, 4.0% to 50% in yet another embodiment, 6.0% to 50% in yet another embodiment, 8.0% to 50% in yet another embodiment, 10% to 50% in one embodiment, 15% to 50% in another embodiment, 20% to 50% in yet another embodiment, 25% to 50% in yet another embodiment, 30% to 50% in yet another embodiment, 40% to 50% in yet another embodiment and 45% to 50% in yet another embodiment of small particles; and in arrange of from 50% to 99.99% in one embodiment, 55% to 99.99% in another embodiment, 60% to 99.99% in yet another embodiment, 65% to 99.99% in yet another embodiment, 70% to 99.99% in yet another embodiment, 75% to 99.99% in yet another embodiment, 80% to 99.99% in yet another embodiment and 85% to 99.99% in yet another embodiment of large particles. In particular embodiments, the pharmaceutical composition can comprise about 0.01% to about 10% of the small particles and about 90% to about 99.99% of the large particles, percentage based on the total counts of the particles measured.

The pharmaceutical composition can comprise a population of particles (e.g., particles of brexanolone), wherein the particles have a mean particle size of about 0.1-50 μm. In some embodiments, the particles have a mean particle size of about 0.1 μm to about 50 μm. In some embodiments, the particles have a mean particle size of at least about 0.1 μm. In some embodiments, the particles have a mean particle size of at most about 50 μm. In some embodiments, the particles have a mean particle size of about 0.1 μm to about 0.2 μm, about 0.1 μm to about 0.5 μm, about 0.1 μm to about 1 μm, about 0.1 μm to about 2 μm, about 0.1 μm to about 5 μm, about 0.1 μm to about 10 μm, about 0.1 μm to about 20 μm, about 0.1 μm to about 30 μm, about 0.1 μm to about 40 μm, about 0.1 μm to about 50 μm, about 0.2 μm to about 0.5 μm, about 0.2 μm to about 1 μm, about 0.2 μm to about 2 μm, about 0.2 μm to about 5 μm, about 0.2 μm to about 10 μm, about 0.2 μm to about 20 μm, about 0.2 μm to about 30 μm, about 0.2 μm to about 40 μm, about 0.2 μm to about 50 μm, about 0.5 μm to about 1 μm, about 0.5 μm to about 2 μm, about 0.5 μm to about 5 μm, about 0.5 μm to about 10 μm, about 0.5 μm to about 20 μm, about 0.5 μm to about 30 μm, about 0.5 μm to about 40 μm, about 0.5 μm to about 50 μm, about 1 μm to about 2 μm, about 1 μm to about 5 μm, about 1 μm to about 10 μm, about 1 μm to about 20 μm, about 1 μm to about 30 μm, about 1 μm to about 40 μm, about 1 μm to about 50 μm, about 2 μm to about 5 μm, about 2 μm to about 10 μm, about 2 μm to about 20 μm, about 2 μm to about 30 μm, about 2 μm to about 40 μm, about 2 μm to about 50 μm, about 5 μm to about 10 μm, about 5 μm to about 20 μm, about 5 μm to about 30 μm, about 5 μm to about 40 μm, about 5 μm to about 50 μm, about 10 μm to about 20 μm, about 10 μm to about 30 μm, about 10 μm to about 40 μm, about 10 μm to about 50 μm, about 20 μm to about 30 μm, about 20 μm to about 40 μm, about 20 μm to about 50 μm, about 30 μm to about 40 μm, about 30 μm to about 50 μm, or about 40 μm to about 50 μm. In some embodiments, the particles have a mean particle size of about 0.1 μm, about 0.2 μm, about 0.5 μm, about 1 μm, about 2 μm, about 5 μm, about 10 μm, about 20 μm, about 30 μm, about 40 μm, or about 50 μm. The particles have a mean particle size of about 1.5-15 μm in one example, about 3-5 μm in another example, about 0.2-1.5 μm in yet another example and about 0.5-0.9 μm in yet another example.

The pharmaceutical composition can comprise at least 50%, 60%, 70%, 80%, or 90% of particles having a particle size of about 0.2-15 μm. In some embodiments, the pharmaceutical composition comprises about 0.01%-50% of the particles having a mean particle size of about 0.2-1.5 μm and about 50% to 99.99% of the particles having a mean particle size of about 1.5-15 μm. The mean particle size of the population of the particles can be measured from one or more samples of the particles based on the total number (counts) of particles measured.

Particle size of the disclosed pharmaceutical compositions can be adjusted (e.g., by milling or other technique known in the art) to change the release profile characteristics of drug in the pharmaceutical compositions of the present disclosure. In some embodiments, the particles may be prepared by grinding/milling. Grinding can take place in any suitable grinding mill. Suitable mills for grinding/milling include an air jet mill, a roller mill, a ball mill, an attritor mill, a vibratory mill, a planetary mill, a sand mill and a bead mill. A high energy media mill is preferred when small particles are desired. The mill can contain a rotating shaft. Particles can also be prepared by wet milling.

In some embodiments, brexanolone of the disclosed aqueous suspension pharmaceutical compositions is crystalline form (polymorph Form A) characterized by having at least 2, 3, 4, 5, 6, 7, 8, or 10 of the following peaks in Powder X-Ray Diffraction (PXRD) diffractograms, at 7.25, 8.88, 11.46, 14.50, 14.78, 17.77, 18.15, 18.32, 18.61 and 19.99±0.1 2θ(°). In some embodiments, the brexanolone crystalline form can have at least two of the aforementioned peaks having relative intensities at or greater than 50%. In some embodiments, the brexanolone crystalline form can have at least two of the aforementioned peaks, at 7.25 and 18.15, having relative intensities at or greater than 50% in one example, at or greater than 60% in another example, at or greater than 70% in yet another example, at or greater than 80% in yet another example or at or greater than 90% in yet another example. The brexanolone polymorph Form A can have particle sizes in a range of 1 μm to 100 μm that can be used for different formulations. In some embodiments, an injectable formulation of a pharmaceutical composition can have the brexanolone polymorph Form A having particle sizes in a range of 1 μm to 15 μm, 1 μm to 14 μm, 1 μm to 13 μm, 1 μm to 12 μm, 1 μm to 11 μm, 1 μm to 10 μm, 1 μm to 9 μm, 1 μm to 8 μm, 1 μm to 7 μm, 1 μm to 6 μm, 1 μm to 5 μm, 1 μm to 4 μm, 1 μm to 3 μm, 1 μm to 2 μm or 1 μm to 1.5 μm. In some embodiments, the aqueous suspension pharmaceutical compositions of the present disclosure comprise a brexanolone crystalline form (polymorph Form A) characterized herein.

In some embodiments, the aqueous suspension pharmaceutical composition comprises a brexanolone polymorph Form A, wherein the brexanolone polymorph Form A can have a chemical purity of greater than 80%, 85%, 90%, 95%, 96%, 97%, 98%, 98% or 99%, percentage based on weight (w/w) of the brexanolone. In some embodiments, the brexanolone polymorph Form A can be quantified by HPLC. In some embodiments, the brexanolone polymorph Form A can have a melting point of about 170-180° C. In some embodiments, the brexanolone polymorph Form A can have a melting point of about 174° C.

The brexanolone crystalline form can be crystalized from one or more solvents selected from the group consisting of dichloromethane (DCM), tetrahydrofuran (THF), ethyl acetate (EtOAc), dimethyl sulfoxide (DMSO), toluene, 2-propanol:water (9:1) (v/v), methanol (MeOH), 2-propanol (IPA), methyl t-butyl ether (MTBE), isopropyl ether (IPE), acetonitrile (MeCN), n-heptane, ethanol, water and a miscible combination thereof. The term “miscible combination” used herein throughout this disclosure means two or more solvents can be mixed together forming a solution without precipitation or phase separation. In some embodiments, the one or more solvent can be free from acetonitrile.

### Pharmaceutically Acceptable Excipients

In some embodiments, the present disclosure provides an aqueous suspension pharmaceutical composition comprising a pharmaceutically effective amount of a neuroactive steroid (e.g., brexanolone), or pharmaceutically acceptable salts or derivatives thereof, and one or more pharmaceutically acceptable excipients.

In some embodiments, the one or more pharmaceutically acceptable excipients comprises one or more surfactants, emulsifiers, fillers, carriers, isotonicifiers, dispersing agents, viscosity modifiers, suspending agent, buffering agents, or combinations thereof. In some embodiments, the one or more pharmaceutically acceptable excipients includes one or more surfactants, one or more suspending agents, one or more tonicity agents, one or more buffering agents, or a combination thereof.

Pharmaceutical acceptable carriers, excipients or inactive ingredients from the Inactive Ingredients Database available from US FDA (https://www.fda.gov/drugs/drug-approvals-and-databases/inactive-ingredients-database-download) can be suitable. Some of Generally Recognized As Safe (GRAS) food substances available form US FDA's GRAS Substances (SCOGS) Database (https://www.fda.gov/food/generally-recognized-safe-gras/gras-substances-scogs-database) can also be suitable.

In some embodiments, the pharmaceutically acceptable excipient is a pharmaceutically acceptable carrier. In embodiments of the present disclosure, the pharmaceutical acceptable excipient/carrier can comprise acacia, animal oils, benzyl alcohol, benzyl benzoate, calcium stearate, carbomers, cetostearyl alcohol, cetyl alcohol, cholesterol, cyclodextrins, dextrose, diethanolamine, emulsifying wax, ethylene glycol palmitostearate, glycerin, glycerin monostearate, glycerol stearate, glyceryl monooleate, glyceryl monostearate, hydrous, histidine, hydrochloric acid, hydroxypropyl cellulose, hydroxypropyl-β-cyclodextrin (HPBCD), hypromellose (hydroxypropyl methylcellulose (HPMC)), lanolin, lanolin alcohols, lecithin, medium-chain triglycerides, metallic soaps, methylcellulose, mineral oil, monobasic sodium phosphate, monoethanolamine, oleic acid, polyethylene glycols (PEG 3350, PEG 4000, PEG 6000), polyoxyethylene-polyoxypropylene copolymer (poloxamer), polyoxyethylene alkyl ethers, polyoxyethylene castor oil, polyoxyethylene castor oil derivatives, polyoxyethylene sorbitan fatty acid esters, polyoxyethylene stearates, polysorbate, polyoxyethylene (20) sorbitan monolaurate (Tween 20, Polysorbate 20), polyoxyethylene (20) sorbitan monooleate (Tween 80, Polysorbate 80), povidone, propylene glycol alginate, saline, sodium chloride, sodium citrate, sodium citrate dihydrate, sodium hydroxide, sodium lauryl sulfate, sodium phosphate monobasic, sodium phosphate dibasic, sorbitan esters, stearic acid, stearyl alcohol, sunflower oil, tragacanth, triethanolamine, vegetable oils, water, xanthan gum, or a combinations thereof.

In some embodiments, the pharmaceutical acceptable carrier is selected from the group consisting of dextrose, glycerin, histidine, hydrochloric acid, hydroxypropyl cellulose, hydroxypropyl-β-cyclodextrin (HPBCD), hypromellose (hydroxypropyl methylcellulose (HPMC)), polyoxyethylene (20) sorbitan monolaurate (Tween 20, Polysorbate 20), polyethylene glycols (PEG 3350, PEG 4000, PEG 6000), polyoxyethylene-polyoxypropylene copolymer (Poloxamer 188, Poloxamer 407), polyoxyethylene (20) sorbitan monooleate (Tween 80, Polysorbate 80), saline, sodium chloride, sodium citrate, sodium citrate dihydrate, sodium lauryl sulfate, sodium phosphate monobasic, sodium phosphate dibasic, and combinations thereof.

In some embodiments, the aqueous suspension pharmaceutical compositions of the present disclosure comprise one or more surfactants. Surfactants, as used herein, can be used in the disclosed compositions as solubilizing agents to increase drug solubility in the formulation. Suitable surfactants may be anionic, cationic, amphoteric or nonionic surface active agents. Suitable anionic surfactants include, but are not limited to, those containing or comprising carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine. Surfactants also include compounds such as lecithin (phosphatides); sorbitan trioleate and other sorbitan esters; polyoxyethylene sorbitan fatty acid esters (e.g., the commercially available TWEENS such as polyoxyethylene sorbitan monolaurate (TWEEN 20, also known as Polysorbate 20, CAS Reg. No. 9005-64-5) and polyoxyethylene sorbitan monooleate (TWEEN 80, also known as Polysorbate 80 (CAS Reg. No. 9005-65-6)); poloxamers (e.g., poloxamer 188 (PLURONIC F68) and poloxamer 338 (PLURONIC F108), which are block copolymers of ethylene oxide and propylene oxide, and poloxamer 407, which is a triblock copolymer of propylene glycol and two blocks of polyethylene glycol); sodium cholesterol sulfate or other cholesterol salts; and bile salts, such as sodium deoxycholate, sodium cholate, sodium glycolate, salts of deoxycholic acid, salts of glycholic acid, salts of chenodeoxycholic acid, and salts of lithocholic acid. In some embodiments, the surfactant is a nonionic surfactant. In some embodiments, the nonionic surfactant comprises a polysorbate. In some embodiments, the nonionic surfactant comprises polysorbate 80.

In some embodiments, the aqueous suspension pharmaceutical composition comprises about 0.2% to about 1.5% by weight of surfactant per volume of composition, e.g., about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, or about 1.5% by weight of surfactant per volume of composition, including all ranges and values therebetween. In some embodiments, the aqueous suspension pharmaceutical composition comprises about 0.2% to about 1.0% by weight of surfactant per volume of composition. In some embodiments, the aqueous suspension pharmaceutical composition comprises about 0.5% to about 0.9% by weight of surfactant per volume of composition. In some embodiments, the aqueous suspension pharmaceutical composition comprises about 0.6% to about 0.8% by weight of surfactant per volume of composition. In some embodiments, the aqueous suspension pharmaceutical composition comprises about 0.6% to about 0.7% by weight of surfactant per volume of composition.

In some embodiments, the aqueous suspension pharmaceutical compositions of the present disclosure comprise one or more suspending (i.e., viscosity modifying) agents. Suitable suspending agents include, but are not limited to, methylcellulose (MC), sodium carboxymethylcellulose (CMC), hydroxypropylmethylcellulose (HPMC) and polyethylene glycols, e.g., higher molecular weight PEGs. In some embodiments, the suspending agent included in the aqueous suspension pharmaceutical compositions of the present disclosure comprises polyethylene glycol (PEG). In some embodiments, the PEG is a higher molecular weight PEG. In some embodiments, the higher molecular weight PEG is PEG 3350, PEG 4000 or PEG 6000. In some embodiments, the higher molecular weight PEG is PEG 3350.

In some embodiments, the aqueous suspension pharmaceutical composition comprises about 0.2% to about 1.5% by weight of suspending agent per volume of composition, e.g., about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, or about 1.5%, including all ranges and values therebetween. In some embodiments, the aqueous suspension pharmaceutical composition comprises about 0.2% to about 1.0% by weight of suspending agent per volume of composition. In some embodiments, the aqueous suspension pharmaceutical composition comprises about 0.5% to about 0.9% by weight of suspending agent per volume of composition. In some embodiments, the aqueous suspension pharmaceutical composition comprises about 0.6% to about 0.8% by weight of suspending agent per volume of composition. In some embodiments, the aqueous suspension pharmaceutical composition comprises about 0.6% to about 0.7% by weight of suspending agent per volume of composition.

In some embodiments, the aqueous suspension pharmaceutical compositions of the present disclosure comprise a tonicity adjusting agent. In some embodiments, the tonicity adjusting agent is selected from the group consisting of sodium chloride, dextrose, mannitol and glycerin. In some embodiments, the tonicity adjusting agent is dextrose or mannitol. In some embodiments, the tonicity adjusting agent is mannitol.

In some embodiments, the aqueous suspension pharmaceutical composition comprises about 2% to about 10% by weight of tonicity adjusting agent per volume of composition, e.g., about 2%, about 3%, about 4%, about 5%, about 6%, about 7%, about 8%, about 9%, or about 10%, including all ranges and values therebetween. In some embodiments, the aqueous suspension pharmaceutical composition comprises about 2% to about 6% by weight of tonicity adjusting agent per volume of composition. In some embodiments, the aqueous suspension pharmaceutical composition comprises about 3% to about 4% by weight of tonicity adjusting agent per volume of composition. In some embodiments, the aqueous suspension pharmaceutical composition comprises about 3.4% by weight of tonicity adjusting agent per volume of composition.

In some embodiments, the aqueous suspension pharmaceutical compositions of the present disclosure comprise a buffering agent, which can be used to adjust and stabilize pH for parenteral (e.g., IM) preparations. Examples of acid buffers include oxalic acid, maleic acid, fumaric acid, lactic acid, malic acid, tartaric acid, citric acid, benzoic acid, acetic acid, methanesulfonic acid, histidine, succinic acid, toluenesulfonic acid, benzenesulfonic acid, ethanesulfonic acid, and the like. In some embodiments, the buffering agent of the present disclosure is a citrate, phosphate, or acetate buffering agent. In some embodiments, the buffering agent is a citrate buffering agent. In some embodiments, the citrate buffering agent comprises sodium citrate dihydrate and citric acid monohydrate. In some embodiments, the pH of the aqueous suspension pharmaceutical composition is in a range of from about 5 to about 9. In some embodiments, the pH of the aqueous suspension pharmaceutical composition is in a range of from about 6 to about 7. In some embodiments, the pH is about 6. In some embodiments, the pH is about 7.

In some embodiments, the aqueous suspension pharmaceutical composition comprises about 0.1% to about 1% by weight of buffering agent per volume of composition, e.g., about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95% or about 1%, including all ranges and values therebetween. In some embodiments, the aqueous suspension pharmaceutical composition comprises about 0.1% to about 0.5% by weight of buffering agent per volume of composition. In some embodiments, the aqueous suspension pharmaceutical composition comprises about 0.1% to about 0.3% by weight of buffering agent per volume of composition.

In some embodiments, the aqueous suspension pharmaceutical composition comprises about 0.1% to about 1% by weight of sodium citrate dihydrate per volume of composition, e.g., about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95% or about 1%, including all ranges and values therebetween. In some embodiments, the aqueous suspension pharmaceutical composition comprises about 0.1% to about 0.5% by weight of sodium citrate dihydrate per volume of composition. In some embodiments, the aqueous suspension pharmaceutical composition comprises about 0.1% to about 0.3% by weight of sodium citrate dihydrate per volume of composition. In some embodiments, the aqueous suspension pharmaceutical composition comprises about 0.17% by weight of sodium citrate dihydrate per volume of composition.

In some embodiments, the aqueous suspension pharmaceutical composition comprises about 0.005% to about 0.02% by weight of citric acid monohydrate per volume of composition, e.g., about 0.005%, about 0.006%, about 0.008%, about 0.010%, about 0.012%, about 0.014%, about 0.016%, about 0.018%, or about 0.02%, including all ranges and values therebetween. In some embodiments, the aqueous suspension pharmaceutical composition comprises about 0.005% to about 0.015% by weight of citric acid monohydrate per volume of composition. In some embodiments, the aqueous suspension pharmaceutical composition comprises about 0.010% by weight of citric acid monohydrate per volume of composition.

In some embodiments, the aqueous suspension pharmaceutical composition comprises brexanolone, and one or more pharmaceutically acceptable excipients selected from the group consisting of a surfactant, a suspending agent, a tonicity adjusting agent, and a buffering agent. In some embodiments, the composition is administered as a single intramuscular injection.

In some embodiments, the aqueous suspension pharmaceutical composition comprises brexanolone, one or more surfactants, one or more suspending agents, a tonicity adjusting agent, and a buffering agent. In some embodiments, the composition is administered as a single intramuscular injection.

In some embodiments of the present disclosure, the aqueous suspension pharmaceutical composition comprises the following unit formula for ER Brexanolone-B:

| Ingredient | Amount/vial (1.0 mL filled) | |
|---|---|---|
| Brexanolone | 100 mg | |
| PEG 3350 | 6.71 mg | (0.67% w/v) |
| Polysorbate 80 | 6.71 mg/mL | (0.67% w/v) |
| Mannitol | 33.54 mg | (3.4% w/v) |
| Sodium citrate dihydrate | 1.72 mg | (0.17% w/v) |
| Citrate acid monohydrate | 0.104 mg | (0.01% w/v) |

| | |
|---|---|
| Water | QS |

In some embodiments of the present disclosure, the aqueous suspension pharmaceutical composition comprises the following unit formula for ER Brexanolone-A:

| Ingredient | Amount/vial (1.3 mL filled) | |
|---|---|---|
| Brexanolone | 390 | mg |
| PEG 3350 | 8.73 | mg |
| Polysorbate 80 | 8.73 | mg |
| Mannitol | 43.60 | mg |
| Sodium citrate dihydrate | 2.23 | mg |
| Citrate acid monohydrate | 0.14 | mg |

| | |
|---|---|
| Water | QS |

In some embodiments, the disclosed compositions further comprise a preservative. The preservative may be used to inhibit bacterial growth or prevent deterioration of the active agent. Preservatives suitable for parenteral formulations include ascorbic acid, acetylcysteine, benzalkonium chloride, benzethonium chloride, benzoic acid, benzyl alcohol, chlorobutanol, chlorhexidine, m-cresol, 2-ethoxyethanol, human serum albumin, monothioglycerol, parabens (methyl, ethyl, propyl, butyl, and combinations), phenol, phenylmercurate salts (acetate, borate nitrate), sorbic acid, sulfurous acid salts (bisulfite and metabisulfite), and thimerosal. In some embodiments the preservative is an antioxidant such ascorbic acid, glutathione, or an amino acid. Amino acids useful as antioxidants include methionine, cysteine, and L-arginine.

In some embodiments, the pharmaceutical composition are substantially free of cyclodextrins. In some embodiments, the pharmaceutical composition are substantially free of sulfobutyl ether β-cyclodextrin. By “substantially free of”, it means the pharmaceutical composition comprises no detectable amount of cyclodextrins or sulfobutyl ether β-cyclodextrin or less than 0.1%, such as less than 0.1% in one example, less than 0.05% in another example, less than 0.01% in yet another example, less than 0.005% in yet another example, less than 0.001% in yet another example, of cyclodextrins or sulfobutyl ether β-cyclodextrin, percent based on the total weight of the pharmaceutical composition.

In some embodiments, the disclosed pharmaceutical composition comprises particles comprising at a neuroactive steroid (e.g., brexanolone) and one or more pharmaceutical acceptable excipients, wherein the neuroactive steroid is a positive modulator of γ aminobutyric acid type A (GABA_{A}) receptors; wherein the particles comprise large particles having a particle size in a range of from about 1.5 μm to about 15 μm and small particles having a particle size in a range of from about 0.2 μm to about 1.5 μm; and wherein about 0.01% to about 50% of the particles are small particles and about 50% to 99.99% of the particles are large particles, wherein the percentage is based on the total counts of the particles measured. In some embodiments, the large particles have a mean particle size in a range of from 2.0 to 6.0 μm in one example, a mean particle size in a range of from 3.0 to 5.0 μm in another example, a mean particle size in a range of from 0.4 to 1.3 μm in yet another example and a mean particle size in a range of from 0.5 to 0.9 μm in a further example. In some embodiments or examples of the pharmaceutical composition, the particles can be stabilized particles disclosed herein.

### Pharmacokinetics

In some embodiments, upon a single intramuscular injection of about 30 mg of brexanolone (100 mg/mL), the pharmaceutical composition of the present disclosure is formulated to provide an average daily AUC of brexanolone that is at least about 10 ng*h/mL/day or more for at least about 168 hours to about 336 hours (e.g., about 168 h, about 192 h, about 216 h, about 240 h, about 264 h, about 288 h, about 312 h, or about 336 h, inclusive of all ranges and values therebetween) following the administration. In some embodiments, the average daily AUC of brexanolone is at least 10 ng*h/mL/day, about 11 ng*h/mL/day, about 12 ng*h/mL/day, about 13 ng*h/mL/day, about 14 ng*h/mL/day, about 15 ng*h/mL/day, about 16 ng*h/mL/day, about 17 ng*h/mL/day, about 18 ng*h/mL/day, about 19 ng*h/mL/day, about 20 ng*h/mL/day, about 21 ng*h/mL/day, about 22 ng*h/mL/day, about 23 ng*h/mL/day, about 24 ng*h/mL/day, about 25 ng*h/mL/day, about 26 ng*h/mL/day, about 27 ng*h/mL/day, about 28 ng*h/mL/day, about 29 ng*h/mL/day, about 30 ng*h/mL/day, about 31 ng*h/mL/day, about 32 ng*h/mL/day, about 33 ng*h/mL/day, about 34 ng*h/mL/day, or about 35 ng*h/mL/day, including all ranges and values therebetween for about 168 h to about 336 h. In some embodiments, the brexanolone has a particle size of about 3 μm.

In some embodiments, upon a single intramuscular injection of about 100 mg of brexanolone (100 mg/mL or 300 mg/mL), the pharmaceutical composition of the present disclosure is formulated to provide an average daily AUC of brexanolone that is at least about 50 ng*h/mL/day or more for at least about 168 hours to about 336 hours (e.g., about 168 h, about 192 h, about 216 h, about 240 h, about 264 h, about 288 h, about 312 h, or about 336 h, inclusive of all ranges and values therebetween) following the administration. In some embodiments, the average daily AUC of brexanolone is at least ng*h/mL/day, about 51 ng*h/mL/day, about 52 ng*h/mL/day, about 53 ng*h/mL/day, about 54 ng*h/mL/day, about 55 ng*h/mL/day, about 56 ng*h/mL/day, about 57 ng*h/mL/day, about 58 ng*h/mL/day, about 59 ng*h/mL/day, about 60 ng*h/mL/day, about 61 ng*h/mL/day, about 62 ng*h/mL/day, about 63 ng*h/mL/day, about 64 ng*h/mL/day, about 65 ng*h/mL/day, about 66 ng*h/mL/day, about 67 ng*h/mL/day, about 68 ng*h/mL/day, about 69 ng*h/mL/day, about 70 ng*h/mL/day, about 71 ng*h/mL/day, about 72 ng*h/mL/day, about 73 ng*h/mL/day, about 74 ng*h/mL/day, about 75 ng*h/mL/day, about 76 ng*h/mL/day, about 77 ng*h/mL/day, about 78 ng*h/mL/day, about 79 ng*h/mL/day, about 80 ng*h/mL/day, about 81 ng*h/mL/day, or about 82 ng*h/mL/day, including all ranges and values therebetween for about 168 h to about 336 h. In some embodiments, the brexanolone has a particle size of about 3 μm.

In some embodiments, upon a single intramuscular injection of about 300 mg of brexanolone (100 mg/mL or 300 mg/mL), the composition of the present disclosure is formulated to provide an average daily AUC of brexanolone that is at least about 200 ng*h/mL/day or more for at least about 168 hours to about 336 hours (e.g., about 168 h, about 192 h, about 216 h, about 240 h, about 264 h, about 288 h, about 312 h, or about 336 h, inclusive of all ranges and values therebetween) following the administration. In some embodiments, the average daily AUC of brexanolone is about 195 ng*h/mL/day, about 196 ng*h/mL/day, about 197 ng*h/mL/day, about 198 ng*h/mL/day, about 199 ng*h/mL/day, 200 ng*h/mL/day, about 201 ng*h/mL/day, about 202 ng*h/mL/day, about 203 ng*h/mL/day, about 204 ng*h/mL/day, about 205 ng*h/mL/day, about 206 ng*h/mL/day, about 207 ng*h/mL/day, about 208 ng*h/mL/day, about 209 ng*h/mL/day, about 210 ng*h/mL/day, about 211 ng*h/mL/day, about 212 ng*h/mL/day, about 213 ng*h/mL/day, about 214 ng*h/mL/day, about 215 ng*h/mL/day, about 216 ng*h/mL/day, about 217 ng*h/mL/day, about 218 ng*h/mL/day, about 219 ng*h/mL/day, about 220 ng*h/mL/day, about 221 ng*h/mL/day, about 222 ng*h/mL/day, about 223 ng*h/mL/day, about 224 ng*h/mL/day, about 225 ng*h/mL/day, about 226 ng*h/mL/day, about 227 ng*h/mL/day, about 228 ng*h/mL/day, about 229 ng*h/mL/day, about 230 ng*h/mL/day, about 231 ng*h/mL/day, about 232 ng*h/mL/day, about 233 ng*h/mL/day, about 234 ng*h/mL/day, about 235 ng*h/mL/day, about 236 ng*h/mL/day, about 237 ng*h/mL/day, about 238 ng*h/mL/day, about 239 ng*h/mL/day, about 240 ng*h/mL/day, about 241 ng*h/mL/day, about 242 ng*h/mL/day, about 243 ng*h/mL/day, about 244 ng*h/mL/day, about 245 ng*h/mL/day, about 246 ng*h/mL/day, about 247 ng*h/mL/day, about 248 ng*h/mL/day, about 249 ng*h/mL/day, or about 250 ng*h/mL/day, including all ranges and values therebetween. In some embodiments, the average daily AUC of brexanolone is maintained for about 168 h to about 336 h. In some embodiments, the brexanolone has a particle size of about 3 μm.

In some embodiments, upon intramuscular injection of from about 30 mg to about 300 mg of brexanolone (100 mg/mL or 300 mg/mL), the composition of the present disclosure is formulated to provide a C_{max,1} of brexanolone of from about 1 ng/mL to about 50 ng/mL, e.g., about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, about 20 ng/mL, about 21 ng/mL, about 22 ng/mL, about 23 ng/mL, about 24 ng/mL, about 25 ng/mL, about 26 ng/mL, about 27 ng/mL, about 28 ng/mL, about 29 ng/mL, about 30 ng/mL, about 31 ng/mL, about 32 ng/mL, about 33 ng/mL, about 34 ng/mL, about 35 ng/mL, about 36 ng/mL, about 37 ng/mL, about 38 ng/mL, about 39 ng/mL, about 40 ng/mL, about 41 ng/mL, about 42 ng/mL, about 43 ng/mL, about 44 ng/mL, about 45 ng/mL, about 46 ng/mL, about 47 ng/mL, about 48 ng/mL, about 49 ng/mL, or about 50 ng/mL, including all ranges and values therebetween. In some embodiments, the brexanolone has a particle size of about 3 μm.

In some embodiments, upon intramuscular injection of from about 30 mg to about 300 mg of brexanolone (100 mg/mL or 300 mg/mL), the composition of the present disclosure is formulated to provide a C_{max,2} of brexanolone of from about 1 ng/mL to about 15 ng/mL, e.g., about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, or about 15 ng/mL, including all ranges and values therebetween. In some embodiments, the brexanolone has a particle size of about 3 μm.

In some embodiments, upon intramuscular injection of from about 30 mg to about 300 mg of brexanolone (100 mg/mL or 300 mg/mL), the composition of the present disclosure is formulated to provide a Cav g of brexanolone over two-weeks of from about 1 ng/mL to about 15 ng/mL, e.g., about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, or about 15 ng/mL, including all ranges and values therebetween. In some embodiments, the brexanolone has a particle size of about 3 μm.

In some embodiments, the aqueous suspension pharmaceutical composition of the present disclosure achieves a mean steady state exposure (Cₛₛ) of brexanolone within the range of about 80% to about 125% of the mean steady state exposure of a reference listed product (e.g., ZULRESSO™) following intramuscular administration. In some embodiments, the composition achieves a mean steady state exposure of brexanolone within the range of about 80% to about 125% of 52 ng/mL to about 79 ng/mL following intramuscular administration.

In some embodiments, when the aqueous suspension pharmaceutical composition comprises about 100 mg of brexanolone, the composition achieves a mean steady state exposure of brexanolone within the range of about 80% to about 125% of 52 ng/mL to about 79 ng/mL following intramuscular administration.

In some embodiments, when the aqueous suspension pharmaceutical composition comprises about 300 mg of brexanolone, the composition achieves a mean steady state exposure of brexanolone within the range of about 80% to about 125% of 52 ng/mL to about 79 ng/mL following intramuscular administration.

In some embodiments, the aqueous suspension pharmaceutical composition of the present disclosure achieves an effective plasma concentration of brexanolone of from about 20 ng/mL to about 80 ng/mL following intramuscular administration. In some embodiments, the composition achieves an effective plasma concentration of brexanolone of from about 45 ng/mL to about 65 ng/mL following intramuscular administration. In some embodiments, the composition achieves an effective plasma concentration of brexanolone of from about 30 ng/mL to about 60 ng/mL following intramuscular administration. In some embodiments, the composition achieves an effective plasma concentration of brexanolone of from about 20 ng/mL to about 50 ng/mL following intramuscular administration. In some embodiments, the composition achieves an effective plasma concentration of brexanolone of about 50 ng/mL following intramuscular administration.

In some embodiments, at least about 50%, 55%, 60%, 65%, 70%, 75%, 80%, or 85% of the effective plasma concentration of brexanolone is maintained for a period greater than about 50 h following intramuscular administration of a single dose of an aqueous suspension pharmaceutical composition disclosed herein. In some embodiments, at least about 40%, 45%, 50%, 55%, 60%, 65%, 70%, or 75% of the effective plasma concentration of brexanolone is maintained for a period greater than about 100 h following intramuscular administration of a single dose of an aqueous suspension pharmaceutical composition disclosed herein. In some embodiments, at least about 30%, 35%, 40%, 45%, 50%, 55%, 60%, or 65% of the effective plasma concentration of brexanolone is maintained for a period greater than about 300 h following intramuscular administration of a single dose of an aqueous suspension pharmaceutical composition disclosed herein.

In some embodiments, the aqueous suspension pharmaceutical composition disclosed herein achieves a mean terminal elimination half-life (T_{1/2}) of brexanolone of greater than about 9 h following intramuscular administration. In some embodiments, the composition achieves a mean terminal elimination half-life of brexanolone of greater than about 10 h following intramuscular administration.

In some embodiments, the neuroactive steroid (e.g., brexanolone) maintains a plasma concentration of more than about 10%, 15%, 20%, 25%, or 30% of the Cₘₐₓ for at least about 3, 4, 5, 6, 7, 8, 9, 10, 20, 30, 40, 50, or 60 days. The neuroactive steroid can maintain a plasma concentration of more than about 10% of the Cₘₐₓ for at least about 10 days in one example, 15% of the Cₘₐₓ for at least about 10 days in another example, 20% of the Cₘₐₓ for at least about 10 days in yet another example, 25% of the Cₘₐₓ for at least about 10 days in yet another example, 35% of the Cₘₐₓ for at least about 10 days in yet another example, 10% of the Cₘₐₓ for at least about 20 days in another example, 15% of the Cₘₐₓ for at least about 20 days in another example, 25% of the Cₘₐₓ for at least about 20 days in yet another example, 35% of the Cₘₐₓ for at least about 20 days in yet another example, 10% of the Cₘₐₓ for at least about 30 days in another example, 15% of the Cₘₐₓ for at least about 30 days in another example, 25% of the Cₘₐₓ for at least about 30 days in yet another example, 35% of the Cₘₐₓ for at least about 30 days in yet another example, 10% of the Cₘₐₓ for at least about 40 days in another example, 15% of the Cₘₐₓ for at least about 40 days in another example, 25% of the Cₘₐₓ for at least about 40 days in yet another example, 35% of the Cₘₐₓ for at least about 40 days in yet another example, 10% of the Cₘₐₓ for at least about 50 days in another example, 15% of the Cₘₐₓ for at least about 50 days in another example, 25% of the Cₘₐₓ for at least about 50 days in yet another example, 35% of the Cₘₐₓ for at least about 50 days in yet another example, 10% of the Cₘₐₓ for at least about 60 days in another example, 15% of the Cₘₐₓ for at least about 60 days in another example, 25% of the Cₘₐₓ for at least about 60 days in yet another example, 35% of the Cₘₐₓ for at least about 60 days in yet another example, 10% of the Cₘₐₓ for at least about 60 or more days in another example, 15% of the Cₘₐₓ for at least about 60 or more days in another example, 25% of the Cₘₐₓ for at least about 60 or more days in yet another example or 35% of the Cₘₐₓ for at least about 60 or more days in yet another example. In one particular example, the neuroactive steroid can maintain a plasma concentration of more than about 15% of the Cₘₐₓ for at least about 30 days. In some embodiments, the plasma concentrations are maintained after a single dose of an aqueous suspension pharmaceutical composition disclosed herein. In some embodiments, the single dose is administered by intramuscular injection.

In some embodiments, the pharmaceutical composition can be administered to the subject one or more times to reach a plasma concentration of the neuroactive steroid in the subject in a range of from 1 ng/mL to about 100 ng/mL. In one embodiment, the neuroactive steroid can be administered one or more times to maintain a plasma concentration in a range of from about 10 ng/mL to about 100 ng/mL. In another embodiment, the neuroactive steroid can be administered one or more times to maintain a plasma concentration in a range of from about 10 ng/mL to about 80 ng/mL. In yet another embodiment, the neuroactive steroid can be administered one or more times to maintain a plasma concentration in a range of from about 10 ng/mL to about 50 ng/mL. In yet another embodiment, the neuroactive steroid can be administered one or more times to maintain a plasma concentration in a range of from about 10 ng/mL to about 40 ng/mL.

In some embodiments, the Cₘₐₓ (e.g., of brexanolone) is about 1 ng/mL to about 100 ng/mL. In some embodiments, the Cₘₐₓ is at least about 1 ng/mL. In some embodiments, the Cₘₐₓ is at most about 100 ng/mL. In some embodiments, the Cₘₐₓ is about 1 ng/mL to about 10 ng/mL, about 1 ng/mL to about 20 ng/mL, about 1 ng/mL to about 40 ng/mL, about 1 ng/mL to about 60 ng/mL, about 1 ng/mL to about 80 ng/mL, about 1 ng/mL to about 100 ng/mL, about 10 ng/mL to about 20 ng/mL, about 10 ng/mL to about 40 ng/mL, about 10 ng/mL to about 60 ng/mL, about 10 ng/mL to about 80 ng/mL, about 10 ng/mL to about 100 ng/mL, about 20 ng/mL to about 40 ng/mL, about 20 ng/mL to about 60 ng/mL, about 20 ng/mL to about 80 ng/mL, about 20 ng/mL to about 100 ng/mL, about 40 ng/mL to about 60 ng/mL, about 40 ng/mL to about 80 ng/mL, about 40 ng/mL to about 100 ng/mL, about 60 ng/mL to about 80 ng/mL, about 60 ng/mL to about 100 ng/mL, or about 80 ng/mL to about 100 ng/mL. In some embodiments, the Cₘₐₓ is about 1 ng/mL, about 10 ng/mL, about 20 ng/mL, about 40 ng/mL, about 60 ng/mL, about 80 ng/mL, or about 100 ng/mL. In particular examples, the Cₘₐₓ is in a range of from 20 to 90 ng/mL. In some embodiments, the aforementioned Cₘₐₓ values are achieved after a single dose of an aqueous suspension pharmaceutical composition disclosed herein. In some embodiments, the single dose is administered by intramuscular injection.

In some embodiments, the single dose is in a range of from 3 mg to about 5 mg per kilogram of body weight, and/or the neuroactive steroid (e.g., brexanolone) maintains a plasma concentration of more than about 10 ng/mL for at least about 5 days. In some embodiments, the neuroactive steroid maintains a plasma concentration of more than 10, 20, 25, 30, 40, 50, 60, 70, 80, 90, 100 ng/mL for at least about 10, 20, 30, 40, 50, or 60 days. In some embodiments, the neuroactive steroid maintains a plasma concentration of more than 10 ng/mL for at least about 10 days in one embodiment, more than 20 ng/mL for at least about 10 days in another embodiment, more than 30 ng/mL for at least about 10 days in yet another embodiment, more than 40 ng/mL for at least about 10 days in yet another embodiment, more than 50 ng/mL for at least about 10 days in yet another embodiment, more than 60 ng/mL for at least about 10 days in yet another embodiment, more than 70 ng/mL for at least about 10 days in yet another embodiment, more than 80 ng/mL for at least about 10 days in yet another embodiment, more than 90 ng/mL for at least about 10 days in yet another embodiment, more than 100 ng/mL for at least about 10 days in yet another embodiment, more than 10 ng/mL for at least about 20 days in one embodiment, more than 20 ng/mL for at least about 20 days in another embodiment, more than 30 ng/mL for at least about 20 days in yet another embodiment, more than 40 ng/mL for at least about 20 days in yet another embodiment, more than 50 ng/mL for at least about 20 days in yet another embodiment, more than 60 ng/mL for at least about 20 days in yet another embodiment, more than 70 ng/mL for at least about 20 days in yet another embodiment, more than 80 ng/mL for at least about 20 days in yet another embodiment, more than 90 ng/mL for at least about 20 days in yet another embodiment, more than 100 ng/mL for at least about 20 days in yet another embodiment, more than 10 ng/mL for at least about 30 days in one embodiment, more than 20 ng/mL for at least about 30 days in another embodiment, more than 30 ng/mL for at least about 30 days in yet another embodiment, more than 40 ng/mL for at least about 30 days in yet another embodiment, more than 50 ng/mL for at least about 30 days in yet another embodiment, more than 60 ng/mL for at least about 30 days in yet another embodiment, more than 70 ng/mL for at least about 30 days in yet another embodiment, more than 80 ng/mL for at least about 30 days in yet another embodiment, more than 90 ng/mL for at least about 30 days in yet another embodiment, more than 100 ng/mL for at least about 30 days in yet another embodiment, more than 10 ng/mL for at least about 40 days in one embodiment, more than 20 ng/mL for at least about 40 days in another embodiment, more than 30 ng/mL for at least about 40 days in yet another embodiment, more than 40 ng/mL for at least about 40 days in yet another embodiment, more than 50 ng/mL for at least about 40 days in yet another embodiment, more than 60 ng/mL for at least about 40 days in yet another embodiment, more than 70 ng/mL for at least about 40 days in yet another embodiment, more than 80 ng/mL for at least about 40 days in yet another embodiment, more than 90 ng/mL for at least about 40 days in yet another embodiment, more than 100 ng/mL for at least about 40 days in yet another embodiment, more than 10 ng/mL for at least about 50 days in one embodiment, more than 20 ng/mL for at least about 50 days in another embodiment, more than 30 ng/mL for at least about 50 days in yet another embodiment, more than 40 ng/mL for at least about 50 days in yet another embodiment, more than 50 ng/mL for at least about 50 days in yet another embodiment, more than 60 ng/mL for at least about 50 days in yet another embodiment, more than 70 ng/mL for at least about 50 days in yet another embodiment, more than 80 ng/mL for at least about 50 days in yet another embodiment, more than 90 ng/mL for at least about 50 days in yet another embodiment, more than 100 ng/mL for at least about 50 days in yet another embodiment, more than 10 ng/mL for at least about 60 days in one embodiment, more than 20 ng/mL for at least about 60 days in another embodiment, more than 30 ng/mL for at least about 60 days in yet another embodiment, more than 40 ng/mL for at least about 60 days in yet another embodiment, more than 50 ng/mL for at least about 60 days in yet another embodiment, more than 60 ng/mL for at least about 60 days in yet another embodiment, more than 70 ng/mL for at least about 60 days in yet another embodiment, more than 80 ng/mL for at least about 60 days in yet another embodiment, more than 90 ng/mL for at least about 60 days in yet another embodiment, more than 100 ng/mL for at least about 60 days in yet another embodiment, more than 10 ng/mL for at least about 60 or more days in one embodiment, more than 20 ng/mL for at least about 60 or more days in another embodiment, more than 30 ng/mL for at least about 60 or more days in yet another embodiment, more than 40 ng/mL for at least about 60 or more days in yet another embodiment, more than 50 ng/mL for at least about 60 or more days in yet another embodiment, more than 60 ng/mL for at least about 60 or more days in yet another embodiment, more than 70 ng/mL for at least about 60 or more days in yet another embodiment, more than 80 ng/mL for at least about 60 or more days in yet another embodiment, more than 90 ng/mL for at least about 60 or more days in yet another embodiment and more than 100 ng/mL for at least about 60 or more days in yet another embodiment. In one further embodiment, the neuroactive steroid maintains a plasma concentration of more than 20 ng/mL for at least about 30 days. In some embodiments, the aforementioned plasma concentrations are maintained after a single dose of an aqueous suspension pharmaceutical composition disclosed herein. In some embodiments, the single dose is administered by intramuscular injection.

In some embodiments, the pharmaceutical composition releases less than about 0.1%, 0.2%, 0.3%, 0.4%, 0.5%, 1%, 2%, 3%, 4%, 5%, 6%, 7%, 8%, 9%, 10%, 15%, 20%, 25%, 30%, 35%, 40%, 45%, or 50% of the neuroactive steroid (e.g., brexanolone) within about 1 hour of the single dose of the pharmaceutical composition by intramuscular or subcutaneous injection. In some embodiments, the pharmaceutical composition releases about 0.1% of the neuroactive steroid (e.g., brexanolone) to about 50% of the neuroactive steroid (e.g., brexanolone) within about 1 hour of the single dose of the pharmaceutical composition administered to the subject by intramuscular or subcutaneous injection. In some embodiments, the pharmaceutical composition releases about 0.1% to about 50% of the neuroactive steroid (e.g., brexanolone) within about 1 hour of the single dose of the pharmaceutical composition administered to the subject by intramuscular or subcutaneous injection. In some embodiments, the pharmaceutical composition releases at most about 50% of the neuroactive steroid (e.g., brexanolone). In some embodiments, the pharmaceutical composition releases about 0.1% to about 0.5%, about 0.1% to about 1%, about 0.1% to about 5%, about 0.1% to about 10%, about 0.1% to about 20%, about 0.1% to about 50%, about 0.5% to about 1%, about 0.5% to about 5%, about 0.5% to about 10%, about 0.5% to about 20%, about 0.5% to about 50%, about 1% to about 5%, about 1% to about 10%, about 1% to about 20%, about 1% to about 50%, about 5% to about 10%, about 5% to about 20%, about 5% to about 50%, about 10% to about 20%, about 10% to about 50%, or about 20% to about 50% of the neuroactive steroid (e.g., brexanolone) within about 1 hour of the single dose of the pharmaceutical composition administered to the subject by intramuscular or subcutaneous injection. In some embodiments, the pharmaceutical composition releases about 0.1%, about 0.5%, about 1%, about 5%, about 10%, about 20%, or about 50% of the neuroactive steroid (e.g., brexanolone) within about 1 hour of the single dose of the pharmaceutical composition administered to the subject by intramuscular or subcutaneous injection. The percentage of release is based on measured plasma concentration of the neuroactive steroid and the total amount of the neuroactive steroid in the single dose of the pharmaceutical composition administered to the subject.

In some embodiments, the pharmaceutical composition has a relative bioavailability (Bioavailability_{IM/SC}/Bioavailability_{IV}) of about 2%-50% at 24 hours after the single dose by intramuscular or subcutaneous injection, in comparison to the same dose by intravenous administration. In some embodiments, the relative bioavailability is about 2% to about 50%. In some embodiments, the relative bioavailability is at least about 2%. In some embodiments, the relative bioavailability is at most about 50%. In some embodiments, the relative bioavailability is about 2% to about 5%, about 2% to about 10%, about 2% to about 20%, about 2% to about 30%, about 2% to about 40%, about 2% to about 50%, about 5% to about 10%, about 5% to about 20%, about 5% to about 30%, about 5% to about 40%, about 5% to about 50%, about 10% to about 20%, about 10% to about 30%, about 10% to about 40%, about 10% to about 50%, about 20% to about 30%, about 20% to about 40%, about 20% to about 50%, about 30% to about 40%, about 30% to about 50%, or about 40% to about 50%. In some embodiments, the relative bioavailability is about 2%, about 5%, about 10%, about 20%, about 30%, about 40%, or about 50%.

In some embodiments, the neuroactive steroid can maintain a plasma concentration in the subject at a level more than about 5%, 10%, 15%, 20%, 25%, 30%, 35%, 40%, 45%, 50%, 55%, or 60% of the Cₘₐₓ for about 1 day to about 100 days. In some embodiments, the neuroactive steroid can maintain a plasma concentration in the subject at a level more than about 5%, 10%, 15%, 20%, 25%, 30%, 35%, 40%, 45%, 50%, 55%, or 60% of the Cₘₐₓ for at least about 1 day. In some embodiments, the neuroactive steroid can maintain a plasma concentration in the subject at a level more than about 5%, 10%, 15%, 20%, 25%, 30%, 35%, 40%, 45%, 50%, 55%, or 60% of the Cₘₐₓ for at most about 100 days. In some embodiments, the neuroactive steroid can maintain a plasma concentration in the subject at a level more than about 5%, 10%, 15%, 20%, 25%, 30%, 35%, 40%, 45%, 50%, 55%, or 60% of the Cₘₐₓ for about 1 day to about 5 days, about 1 day to about 10 days, about 1 day to about 20 days, about 1 day to about 30 days, about 1 day to about 50 days, about 1 day to about 100 days, about 5 days to about 10 days, about 5 days to about 20 days, about 5 days to about 30 days, about 5 days to about 50 days, about 5 days to about 100 days, about 10 days to about 20 days, about 10 days to about 30 days, about 10 days to about 50 days, about 10 days to about 100 days, about 20 days to about 30 days, about 20 days to about 50 days, about 20 days to about 100 days, about 30 days to about 50 days, about 30 days to about 100 days, or about 50 days to about 100 days. In some embodiments, the neuroactive steroid can maintain a plasma concentration in the subject at a level more than about 5%, 10%, 15%, 20%, 25%, 30%, 35%, 40%, 45%, 50%, 55%, or 60% of the Cₘₐₓ for about 1 day, about 5 days, about 10 days, about 20 days, about 30 days, about 50 days, or about 100 days.

### Administration

In some embodiments, the pharmaceutical composition is suitable for intravenous, intramuscular, subcutaneous, parenteral, spinal or epidermal administration (e.g., by injection or infusion). In some embodiments, the pharmaceutical composition is suitable for intramuscular administration. Depending on the route of administration, the active ingredient can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. The pharmaceutical composition can be in the form of sterile aqueous solutions or dispersions. The pharmaceutical composition can also be formulated in a microemulsion, liposome, or other ordered structure suitable to high drug concentration.

In some embodiments, the pharmaceutical composition is administered to a subject in single dose intramuscular (IM) injection, subcutaneous (SC) injection, or a combination thereof, such as in a bolus injection, in a continuous single injection, or a combination thereof. The pharmaceutical composition can be administered to a subject within a time period in a range of from 1 second to about 180 minutes. The pharmaceutical composition can be administered to a subject within a time period in a range of from about 1 second to about 180 minutes in one example, 1 minute to about 180 minutes in another example, 5 minutes to about 180 minutes in yet another example, 10 minutes to about 180 minutes in yet another example, 20 minutes to about 180 minutes in yet another example, 40 minutes to about 180 minutes in yet another example, 50 minutes to about 180 minutes in yet another example, 60 minutes to about 180 minutes in yet another example, or any time one value within the range. In further examples, the pharmaceutical composition can be administered to a subject within a time period in a range of from 1 second to about 150 minutes, 1 second to about 100 minutes, 1 second to about 80 minutes, 1 second to about 60 minutes, 1 second to about 30 minutes, 1 second to about 10 minutes, 1 second to about 5 minutes and 1 seconds to about 1 minute in yet another example. In a particular example, the pharmaceutical composition can be administered to a subject with one shot single injection.

In some cases, the pharmaceutical composition comprising a pharmaceutically effective amount of a neuroactive steroid can be administered to a subject in one or more injections, such as in at least 1, 2, 3, 4, 5, 6, 7, 8, 9, or 10 single dose injections, via intramuscular (IM) injection, subcutaneous (SC) injection, or a combination thereof. The one or more injections can be administered with a specified time interval, such as one injection every 1 hour, 2 hours, 3 hours, 4 hours, 5 hours, 6 hours, 12 hours, 24 hours, 36 hours, 48 hours, 3 days, 4, days, 5 days, 6 days, 7 days, 2 weeks, 3 weeks, 4 weeks, 1 month, 2 months, 3 months, 4 months, 5 months, or 6 months. In some cases, the pharmaceutical composition can be administered to a subject in one single dose IM or SC injection every 6 months. In some cases, the pharmaceutical composition can be administered to a subject in one single dose IM or SC injection every 60 days.

### Methods of Treatment and Prevention

The present disclosure provides a method of treating or preventing a neurological condition in a subject in need thereof, said method comprising administering to the subject a therapeutically effective dose of the pharmaceutical composition disclosed herein.

In some embodiments, the neurological condition is selected from the group consisting of traumatic brain injury, Alzheimer's disease, Parkinson disease, mild cognitive impairment (MCI), epilepsy, seizures, focal onset seizures, PCDH19 pediatric epilepsy, pediatric genetic epilepsies, CDKL5 Deficiency Disorder (CDD), catamenial epilepsy, infantile spasms, anxiety, fragile X tremor-ataxia syndrome, lysosomal storage disorders (Niemann-Pick type C disease), post-traumatic stress disorder (PTSD), postpartum depression (PPD), major depressive disorder (MDD), premenstrual dysphoric disorder (PMDD), persistent depressive disorder (PDD), bipolar disorder, seasonal affective disorder (SAD), secondary depression, postfinasteride syndrome, alcohol craving, smoking cessation, mood disorder, schizophrenia, and other neurologic, psychiatric or neuromuscular disorders. In some embodiments, the neurological condition is postpartum depression (PPD).

In some embodiments, the present disclosure provides a method of treating or preventing postpartum depression in a subject in need thereof, said method comprising administering to the subject a therapeutically effective dose of the pharmaceutical composition disclosed herein.

In some embodiments, the pharmaceutical composition is administered to said subject via intramuscular (IM) injection. In some embodiments, the pharmaceutical composition comprises about 30 mg to about 1000 mg of brexanolone, e.g., about 30 mg; about 50 mg, about 100 mg, about 150 mg, about 200 mg, about 250 mg, about 300 mg, about 400 mg, about 500 mg, about 600 mg, about 700 mg, about 800 mg, about 900 mg, or about 1000 mg, including all ranges and values therebetween. In some embodiments, the pharmaceutical composition comprises about 30 mg to about 500 mg of brexanolone. In some embodiments, the pharmaceutical composition comprises about 30 mg to about 300 mg of brexanolone. In some embodiments, 30 mg of brexanolone are administered by intramuscular injection once every two weeks. In some embodiments, 30 mg of brexanolone are administered by intramuscular injection once every 4 weeks. In some embodiments, 30 mg of brexanolone are administered by intramuscular injection once every 6 weeks. In some embodiments, 100 mg of brexanolone are administered by intramuscular injection once every two weeks. In some embodiments, 100 mg of brexanolone are administered by intramuscular injection once every 4 weeks. In some embodiments, 100 mg of brexanolone are administered by intramuscular injection once every 6 weeks. In some embodiments, 300 mg of brexanolone are administered by intramuscular injection once every 2 weeks. In some embodiments, 300 mg of brexanolone are administered by intramuscular injection once every 4 weeks. In some embodiments, 300 mg of brexanolone are administered by intramuscular injection once every 6 weeks.

In some embodiments of the present methods, the administering comprises: (a) administering an initial dose of the pharmaceutical composition of the present disclosure to a subject; and (b) optionally, administering a second dose or subsequent dose of the pharmaceutical composition of the present disclosure, wherein the second dose or subsequent doses are administered at a timepoint deemed necessary to maintain a therapeutically effective plasma concentration of brexanolone.

In some embodiments, the initial dose of brexanolone and subsequent dose(s) are the same. In some embodiments, the dose comprises about 30 mg to about 500 mg of brexanolone, e.g., about 30 mg; about 50 mg, about 100 mg, about 150 mg, about 200 mg, about 250 mg, about 300 mg, about 400 mg, about 500 mg, inclusive of all ranges and values therebetween. In some embodiments, the dose comprises 30 mg of brexanolone. In some embodiments, the dose comprises 100 mg of brexanolone. In some embodiments, the dose comprises 300 mg of brexanolone.

In some embodiments, the initial dose of brexanolone and subsequent dose(s) are different. In some embodiments, the initial dose of brexanolone is greater than a subsequent dose. In some embodiments, the initial dose comprises about 30 mg to about 500 mg of brexanolone, e.g., about 30 mg; about 50 mg, about 100 mg, about 150 mg, about 200 mg, about 250 mg, about 300 mg, about 400 mg, about 500 mg, inclusive of all ranges and values therebetween. In some embodiments, the initial dose comprises 30 mg of brexanolone. In some embodiments, the initial dose comprises 100 mg of brexanolone. In some embodiments, the initial dose comprises 300 mg of brexanolone.

The subject of the present disclosure can be in lactation, for example, a woman who is lactating, nursing or breastfeeding. The subject can be a woman in lactation after pregnancy. The subject can also be a woman in induced lactation. Based on CDC guidelines (https://www.cdc.gov/breastfeeding/breastfeeding-special-circumstances/maternal-or-infant-illnesses/postpartum-depression.html) and a recent study, allopregnanolone (ALLO) concentrations in milk and plasma are low and that the BRX is associated with low risk to breastfed infants (Hoffmann, et al., Obstetrics & Gynecology, Volume 133, p 1155, May 2019, and Hoffmann, et al., American Journal of Obstetrics & Gynecology, 5554 Supplement to JANUARY 2019).

In some embodiments, the subject can be in a range of from 1 day to 24 months postpartum. In some embodiments, the subject can be a woman 1 day to 12 months after giving birth to a child. In some embodiments, the subject can be breastfeeding an infant every 1 to 6 hours, every 1 to 5 hours, every 1 to 4 hours, every 1 to 3 hours or every 1 to 2 hours.

In some embodiments, each of the single doses is administered to the subject between the pre-admin breastfeeding and the consecutive post-admin breastfeeding of the subject, wherein the consecutive post-admin breastfeeding is in a range of from about 30 minutes to about 360 minutes after the completion of the pre-admin breastfeeding. The consecutive post-admin breastfeeding can be in a range of from about 30 minutes to about 360 minutes, 30 minutes to about 300 minutes, 30 minutes to about 240 minutes, 30 minutes to about 180 minutes, or 30 minutes to about 120 minutes, or 30 minutes to about 60 minutes, after the completion of the pre-admin breastfeeding.

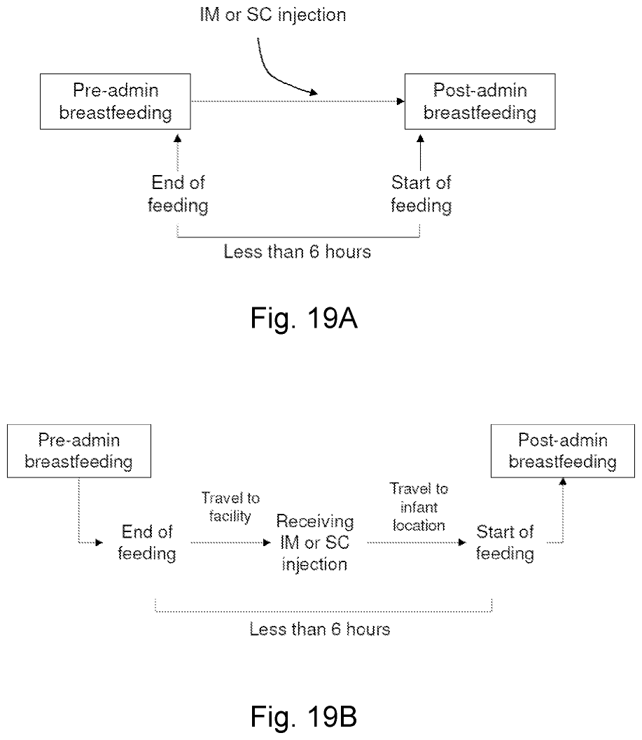

Representative illustrations of examples of treatment schedule are shown in FIG. 19A. In some cases, a woman can provide a pre-admin breastfeeding to her infant at home. After the pre-admin breastfeeding, the woman can go to a facility to receive an administration of a single dose of the pharmaceutical composition via intramuscular (IM) injection or subcutaneous (SC) injection. The injection can be completed in a few minutes, such as in a range of from a few seconds to 2-5 minutes. In some cases, the single dose can be administered to the woman in 1 second to 1 minute, in 1 minute, in 2 minutes, in 3 minutes, in 4 minutes, or in 5 minutes. After the injection, the woman can travel back home or a place where the infant is located. The woman can provide a post-admin breastfeeding for the infant, such as less than 6 hours from the end of the pre-admin breastfeeding (FIG. 19B).

In some cases, disclosed herein is a use of particles comprising at least one neuroactive steroid and one or more pharmaceutical acceptable excipients for manufacturing a medicament for treating a disease, wherein said disease comprises postpartum depression (PPD) in a lactating subject; wherein the medicament is administered to the subject in one or more single doses via intramuscular (IM) injection, subcutaneous (SC) injection, or a combination thereof, wherein each of the single doses is administered to the subject in a time period in a range of from a few seconds to about 30 minutes; wherein the neuroactive steroid is a positive modulator of gamma-aminobutyric acid (GABA) A receptor; wherein each of the single doses is administered to the subject between a pre-admin breastfeeding and a consecutive post-admin breastfeeding of the subject; and wherein each of the single doses is administered to the subject in a range of from 1 minute to about 360 minutes after completion of the pre-admin breastfeeding and about 5 minutes to about 360 minutes before starting the post-admin breastfeeding.

Shortly after giving birth, both progesterone and ALLO levels drop precipitously. It has been hypothesized that this abrupt drop may trigger PPD in vulnerable women. The hypothesis that the rapid reduction in ALLO at parturition could, at least in part, increase the risk for PPD in vulnerable women has led to the development of short-term ALLO replacement therapy for the treatment of PPD. Without being bound by any particular theory, administering the extended-release aqueous suspension pharmaceutical compositions disclosed herein provides a long duration of an effective amount of brexanolone that may avoid a rapid drop-off of physiological allopregnanolone level that is believed to at least contribute to the onset of PPD.

In some embodiments, the present disclosure is directed to a method of preventing postpartum depression (PPD) in a subject in need thereof, the method comprising: obtaining or causing to obtain depression assessment data of the subject, wherein the depression assessment data comprise depression diagnostic data and pregnancy data of the subject; producing risk prediction data based on the depression assessment data; administering a pharmaceutical composition comprising a pharmaceutically effective amount of a neuroactive steroid (e.g., an aqueous suspension pharmaceutical composition disclosed herein) to the subject prior to clinical onset of the PPD if the risk prediction data indicate a high risk of PPD in the subject, wherein the neuroactive steroid is a positive modulator of gamma-aminobutyric acid (GABA) A receptor; and wherein the subject is not diagnosed with PPD at the time the depression assessment data is obtained.

The depression assessment data of the subject, such as a patient, can be obtained by a medical professional such as a doctor, a nurse, a nurse practitioner, or other qualified medical professionals. Some part of the depression assessment data can also be obtained by the patient or another person under the guidance of a medical professional. The pregnancy data can comprise indications or medical examination results of the pregnancy, such as urine test results, hormonal test results, blood tests results, ultrasound examination results, stage of the pregnancy, expected delivery time, and health and vital data of the subject. The depression diagnostic data can comprise historic and present depression diagnostic and examination results and data.

In some embodiments, the depression diagnostic data can comprise historic depression diagnostic data if any, depression data from previous pregnancy if any, present depression diagnostic data, historic Beck's Depression Inventory (BDI) value, present BDI value, historic Edinburgh Postnatal Depression Scale (EPDS) value, present EPDS value, historic Postpartum Depression Predictors Inventory (PDPI), present PDPI value, historic SIGH-ADS29 assessment value, present SIGH-ADS29 assessment value, historic Structured Clinical Interview for DSM-IV (SCID) assessment, present SCID assessment, historic Inventory of Depressive Symptomatology (IDS) assessment, present IDS assessment, historic Quick Inventory of Depressive Symptomatology (QIDS) assessment, present QIDS assessment, clinician IDS (IDS-C), clinician QIDS (QIDS-C), patient self-rated IDS (IDS-SR), patient self-rated QIDS (QIDS-SR), of the subject, or a combination thereof.

It is understood that should the subject be diagnosed with postpartum depression (PPD) at the time the depression assessment data is obtained, the subject should be cared for by qualified medical professionals and receive appropriate treatment determined necessary by those qualified medical professionals.

In some embodiments, the subject is not diagnosed with PPD at the time the depression assessment data is obtained and the risk prediction data can be produced based on the depression assessment data.

In some embodiments, pregnant women at high risk for developing PPD can be identified. Examples of risk factors for the development of PPD can include: social class, life stressors during pregnancy, complicated pregnancy/birth, difficult relationship with family or partner, lack of support from family or friends, prior history of psychopathology (depression, anxiety), chronic stressors postpartum (this can include problems with child care and difficult infant temperament), unemployment or instability, unplanned pregnancy, ambivalence over becoming a pregnant, poor relationship with own mother, history of sexual abuse, lack of a confidante, bottle feeding, depression during pregnancy (this being the strongest predictor of PPD), among others. Risk factors described by Osbourne, et al. (Osbourne, et al., Arch Womens Ment Health, 18(1):41-60, 2015 (ePub 2014), doi: 10.1007/s00737-014-0475-y 2014) can be suitable. Some data indicate that a history of prior PPD can be a strong indicator of recurrent PPD in a subsequent pregnancy, occurring in 26-57% of subsequent births (Munk-Olsen, JAMA Psychiatry. 7(2):213-214, 2020 (2019 online), doi:10.1001/jamapsychiatry.2019.3208).

Several methods and scales have been developed to identify women at risk of PPD (Fisher S D, et al., Depress Anxiety. 2019; 36:375-383. https://doi.org/10.1002/da.22879; Moreira M W L, et al., Information Fusion, 47:23-31, May 2019, https://doi.org/10.1016/j.inffus.2018.07.001). Postpartum Depression Predictors Inventory (PDPI) can be a comprehensive, covering more risk areas than other methods (Beck, J Obstet Gynecol Neonatal Nurs., 31(4):394-402, 2002, doi: 10.1111/j.1552-6909.2002.tb00061.x). Inventory of Depressive Symptomatology (IDS) and the Quick Inventory of Depressive Symptomatology (QIDS), such as the 30-item IDS and the 16-item QIDS, that are designed to assess the severity of depressive symptoms can be used. Both the IDS and the QIDS are available in the clinician (IDS-C and QIDS-C) and self-rated versions (IDS-SR and QIDS-SR).

In some embodiments, risk prediction data can be produced based one the depression diagnostic data comprising historic depression diagnostic data if any, depression data from previous pregnancy if any, present depression diagnostic data, historic Beck's Depression Inventory (BDI) value, present BDI value, historic Edinburgh Postnatal Depression Scale (EPDS) value, present EPDS value, historic Postpartum Depression Predictors Inventory (PDPI), present PDPI value, of the subject, historic SIGH-ADS29 assessment value, present SIGH-ADS29 assessment value, historic Structured Clinical Interview for DSM-IV (SCID) assessment, present SCID assessment, historic Inventory of Depressive Symptomatology (IDS) assessment, present IDS assessment, historic Quick Inventory of Depressive Symptomatology (QIDS) assessment, present QIDS assessment, clinician IDS (IDS-C), clinician QIDS (QIDS-C), patient self-rated IDS (IDS-SR), patient self-rated QIDS (QIDS-SR), other scales or method used to assess depression via a clinician administered or patient-completed scale or report for diagnosis or risk prediction, or a combination thereof.

The depression assessment data can be obtained or caused to be obtained during pregnancy, in a range of from 10 weeks to 0 day prior to the completion of pregnancy, in a range of from 0 day to 24 weeks after completion of pregnancy, of the subject, or a combination thereof. Completion of pregnancy can be giving birth or any other ways of completion of the pregnancy of the subject.

In some embodiments, the depression assessment data can be obtained first during pregnancy (herein referred to as “first depression assessment data”) and then after delivery (herein referred to as “second depression assessment data”). The first depression assessment data can be obtained any time during pregnancy, such as during the first trimester in one example, during the second trimester in another example, and during the third trimester in yet another example. The second depression assessment data can be any time after delivery, such as at 1 week after delivery in one example, 2 to 4 weeks after delivery in another example, 4 to 8 weeks after delivery in yet another example, 8 to 12 weeks after delivery in yet another example, 12 to 16 weeks after delivery in yet another example, 16 to 24 weeks after delivery in yet another example, 20 to 24 weeks after delivery in yet another example, at 24 weeks after delivery, 24 to 28 weeks after delivery in yet another example, or 28 to 48 weeks after delivery in yet another example.

In some embodiments, the risk prediction data can be produced based on the same or different methods. In one example, BDI values can be used in antepartum depression evaluation during pregnancy, and EPDS values can be used on postpartum depression evaluation. In some embodiments, BDI values greater than 6 during pregnancy can be selected as an indication of high risk of developing PPD. In some embodiments, historical depression from previous pregnancy can be an indication of high risk of developing PPD.

In some embodiments, real time behavior monitoring of the subject can be conducted to obtain real time depression assessment data. The data can be compared to a mood disorder database to produce risk prediction data of a subject in real time, during pregnancy, after pregnancy, or a combination thereof. Wearable system with integrated electrodes and sensors capable to acquiring physiological data and body posture information to pattern recognition can be used for obtaining real time depression assessment data. Devices and processes described by Moreira, M W L. et al. (Information Fusion, 2018, doi: 10.1016/j.inffus.2018.07.001) can be suitable. In some embodiments, progressively increasing mood disorder instability can be an indication of high risk of developing PPD. In some embodiments, the wearable system can comprise a smart phone, an App that is operated on a smart phone, iPhone, iWatch, iPad, a laptop, a computer, a wearable digital device, or a combination thereof.

In some embodiments, Structured Clinical Interview for DSM-IV (SCID) can be conducted to obtain depression assessment data and can utilize the SIGH-ADS29, a 29-item, clinician-administered depression assessment that can be used to assess symptom severity known as “Structured interview guide for the Hamilton Depression Rating Scale with Atypical Depression Supplement” (SIGH-ADS). Methods described by Williams & Terman (New York: New York State Psychiatric Institute, 2003) and Wisner et al. (Journal of Clinical Psychiatry, 78:1369-1375, 2017) can be suitable. In some embodiments, progressively increasing severity of SIGH-ADS29 assessment can be an indication of high risk of developing PPD.

In some embodiments, the methods of the present disclosure comprise obtaining or causing to obtain a subsequent depression assessment data of the subject, producing a subsequent risk prediction data based on the depression assessment data and the subsequent depression assessment data, and adjusting dosage of the pharmaceutical composition for administering a subsequent effective amount of a neuroactive steroid to the subject. The subsequent depression assessment data of the subject can be obtained, in a range of from 1 to 24 weeks after the depression assessment data is obtained. The subsequent depression assessment data can comprise subsequent depression diagnostic data and subsequent pregnancy data of the subject. If the subject is diagnosed with clinical onset of PPD based on the subsequent depression assessment data, the dosage of the pharmaceutical composition shall be adjusted suitable for the treatment of PPD. If the subsequent depression assessment data indicates progression of the development of PPD, the dosage of the pharmaceutical composition may be adjusted to increase as determined by qualified medical professionals. If the subsequent depression assessment data indicates no progression of the development of PPD, the dosage of the pharmaceutical composition may be maintained or adjusted to decrease as determined by qualified medical professionals.

In some embodiments, the present method provides a therapeutic effect (e.g., as measured by reduction in Hamilton Depression Score (HAM-D)) within 4, 3, 2, 1 days; 96, 84, 72, 60, 48, 24, 20, 16, 12, 10, 8 hours or less.

In some embodiments, the therapeutic effect is measured by a decrease from baseline in HAM-D score at the end of a treatment period (e.g., 12, 24, 48 hours after administration; 24, 48, 72 hours or more). In some embodiments, the decrease from baseline in HAM-D score is from severe (e.g., HAM-D score of 24 or greater) to symptom-free (e.g., HAM-D score of 7 or lower; remission). In some embodiments, the baseline score is about 10 to 52 (e.g., more than 10, 15, or 20; 10 to 52, 12 to 52, 15 to 52, 17 to 52, 20 to 52, 22 to 52). In some embodiments, the baseline score is at least 10, 15, or 20. In some embodiments, the HAM-D score at the end of the treatment period is about 0 to 10 (e.g., less than 10; 0 to 10, 0 to 6, 0 to 4, 0 to 3, 0 to 2, 1.8). In some embodiments, the HAM-D score at the end of the treatment period is less than 10, 7, 5, or 3. In some embodiments, the decrease in HAM-D score is from a baseline score of about 20 to 30 (e.g., 22 to 28, 23 to 27, 24 to 27, 25 to 27, 26 to 27) to a HAM-D score at the end of the treatment period is about 0 to 10 (e.g., less than 10; 0 to 10, 0 to 6, 0 to 4, 0 to 3, 0 to 2, 1.8). In some embodiments, the decrease in the baseline HAM-D score to HAM-D score at the end of the treatment period is at least 1, 2, 3, 4, 5, 7, 10, 25, 40, 50, or 100 fold). In some embodiments, the percentage decrease in the baseline HAM-D score to HAM-D score at the end of the treatment period is at least 50% (e.g., 60%, 70%, 80%, 90%). In some embodiments, the therapeutic effect is a decrease from baseline in HAM-D score at the end of a treatment period (e.g., 12, 24, 48 hours after administration; 24, 48, 72 hours or more) at least 10, 15, or 20 points. In some embodiments, the therapeutic effect is a decrease from baseline in HAM-D score at the end of a treatment period (e.g., 12, 24, 48 hours after administration; 24, 48, 72 hours or more) at least 5, 7, or 10 points more relative to the therapeutic effect provided by a placebo treatment.

In some embodiments, after administering an initial or single dose, the patient experiences a reduction of depression that is characterized by at least a four point decline in total Hamilton Depression Rating Scale (HAM-D) value. In some embodiments, after administering an initial or single dose, the patient experiences a reduction of depression that is characterized by at least a 40% reduction in HAM-D value. In some embodiments, after administering an initial or single dose, the patient experiences a reduction of depression that is characterized by HAM-D remission. In some embodiments, after an initial or single dose, the patient experiences a reduction of depression that is characterized by an at least two category change in HAM-D severity classification.

In some embodiments, the present method provides therapeutic effect (e.g., as measured by reduction in Montgomery-Asberg Depression Rating Scale (MADRS)) within 4, 3, 2, 1 days; 96, 84, 72, 60, 48, 24, 20, 16, 12, 10, 8 hours or less. The Montgomery-Asberg Depression Rating Scale (MADRS) is a ten-item diagnostic questionnaire (regarding apparent sadness, reported sadness, inner tension, reduced sleep, reduced appetite, concentration difficulties, lassitude, inability to feel, pessimistic thoughts, and suicidal thoughts) which psychiatrists use to measure the severity of depressive episodes in patients with mood disorders. 0-6 indicates normal/symptom absent; 7-19 indicates mild depression; 20-34 indicates moderate depression; and >34 indicates severe depression. In some embodiments, the therapeutic effect is a decrease from baseline in MADRS score at the end of a treatment period (e.g., 12, 24, 48 hours after administration; 24, 48, 60, 72, 96 hours or more). In some embodiments, the decrease from baseline in MADRS score is from severe (e.g., MADRS score of 30 or greater) to symptom-free (e.g., MADRS score of 20 or lower). For example, the mean change from baseline in MADRS total score from treatment with a compound described herein is about −15, −20, −25, −30, while the mean change from baseline in MADRS total score from treatment with placebo is about −15, −10, −5.

In some embodiments, after administering an initial or single dose, the patient experiences a reduction of depression that is characterized by at least a two point decline in Montgomery Asberg Depression Rating Scale (MADRS) value. In some embodiments, after administering an initial or single dose, the patient experiences a reduction of depression that is characterized by at least a 40% reduction in MADRS value. In some embodiments, after administering an initial or single dose, the patient experiences a reduction of depression that is characterized by MADRS remission.

In some embodiments, the present method provides a therapeutic effect (e.g., as measured by reduction in Edinburgh Postnatal Depression Scale (EPDS)) within 4, 3, 2, or 1 days; or 24, 20, 16, 12, 10, or 8 hours or less. In some embodiments, the therapeutic effect is an improvement measured by the EPDS.

In some embodiments, the subject is identified to be at risk through a screening method (e.g., Edinburgh Postnatal Depression Scale (EPDS), e.g., a score of 10 or more on the EPDS, a score of 13 or more on the EPDS).

In some embodiments, the subject is identified to be at risk through screening instruments such as Patient Health Questionnaire (PHQ) in various forms or the Hospital Anxiety and Depression Scales or Geriatric Depression Scale.

In some embodiments, the method provides therapeutic effect (e.g., as measured by reduction in Clinical Global Impression-Improvement Scale (CGI)) within 4, 3, 2, 1 days; 24, 20, 16, 12, 10, 8 hours or less. In some embodiments, the therapeutic effect is a CGI score of 2 or less.

In some embodiments, after administering an initial or single dose, the patient experiences a reduction of depression that is characterized by at least one point decline, a two point decline, or a three point decline in one or more of the Clinical Global Impression (CGI) subscale scores, wherein the CGI subscales are selected from Severity of Illness Subscale (CGI-S) or Global Improvement Subscale (CGI-0.

In some embodiments, after administering an initial or single dose, the patient experiences a reduction of depression that is characterized by at least about a 10%, 20%, 30%, 40% or 50% improvement in Symptoms of Depression Questionnaire (SDQ) total scale score or in any of the respective subscales of SDQ-1, SDQ-2, SDQ-3, SDQ-4 and SDQ-5.

In some embodiments, after administering an initial or single dose, the patient experiences a reduction of depression that is characterized by an at least one point decline, two point decline or three point decline in Pittsburgh Sleep Quality Index (PSQI) Global score.

### Methods of Manufacture

The pharmaceutical compositions disclosed herein can be produced by a process comprising: a) mixing a composition comprising the neuroactive steroid with one or more pharmaceutically acceptable excipients; and b) milling the composition to produce a population of particles to produce the pharmaceutical composition. In some cases, the process can comprise: a) milling a composition comprising the neuroactive steroid to produce a population of particles; and b) mixing the composition with one or more pharmaceutically acceptable excipients to produce the pharmaceutical composition.

In some cases, the pharmaceutical composition comprising particles can be produced by a process comprising: producing a particle mixture comprising at least one neuroactive steroid and one or more pharmaceutical acceptable excipients; milling a first portion of the particle mixture to produce a large particle mixture, wherein at least 50% of the large particle mixture are large particles having a particle size in a range of from about 1.5 μm to about 15 μm, percentage based on the total weight of the particle mixture; and producing the pharmaceutical composition comprising the particles comprising about 50% to 99.99% of the large particles, percentage based on the total weight of the particles measured. The sizes of the particles can also be measured using methods known to in the industry, such as light scattering, and the percentage can be based on the total counts of particles measured.

Commercially available or proprietary neuroactive steroids API can be suitable as a starting material for producing the particle mixture. Typically, the commercially available neuroactive steroids API can have a large particle size. For example, a commercial brexanolone can have a particle size of about 7 to 10 μm in diameter. In another example, a commercial ganaxolone can have a particle size of about 40 to 50 μm. The milling process can reduce particles to a range of suitable sizes.

Typical milling media, such milling beads can be used for milling the particles. The milling bead can have a diameter of 0.1 mm to about 1 mm. In examples, a rotary milling process with a rotation speed of 300 to 600 rpm can be suitable. The particles can be milled for 10 to 40 minutes, 10 to 40 cycles or a time and cycles sufficient to produce particles of desired size range. The milling can be conducted in the presence of one or more excipients disclosed herein.

The large particles can have a mean particle size in a range of from 1.5 μm to about 15 μm in one example, 1.5 μm to 10 μm in another example, 1.5 μm to 8,000 μm in yet another example, 1.5 μm to 6.0 μm in yet another example and 1.5 μm to 4.5 μm in yet another example. In additional examples, the large particles can have a mean particle size in a range of from 2.0 to 6.0 μm. In further embodiments, the large particles can have a mean particle size in a range of from 2.0 to 5.0 μm. In one further example, the large particles can have a particle size of about 2.0 μm to about 4.5 μm.

The process or method can further comprise: milling a second portion of the particle mixture to produce a small particle mixture, wherein the small particle mixture comprises small particles having a particle size in a range of from about 0.2 μm to about 1.5 μm. In some cases, the pharmaceutical composition is produced by mixing the large particle mixture and the small particle mixture to form the particles comprising about 50% to 99.99% of the large particles and 0.01% to 50% of the small particles, percentage based on the total counts of the particles measured.

The first portion and the second portion can be the same or different. In some examples, the first portion and the second portion are the same and the particle mixture is configured to be milled to comprise the large particles and the small particles. In some further examples, the second portion can a part of the first portion and further milled to produce the small particles. In yet some examples, the first portion and the second portion are divided from the original particle mixture and milled separately to produce the large particle and the small particles, respectively.

The small particles can have a mean particle size in a range of from 0.2 μm to about 1.5 μm in one example, 0.2 μm to 1.2 μm in another example, 0.2 μm to 1.0 μm in yet another example, 0.2 μm to 0.8 μm in yet another example and 0.2 μm to 0.7 μm in yet another example. In further examples, the small particles can have a mean particle size in a range of from 0.4 to 1.3 μm. In additional examples, the small particles can have a mean particle size in a range of from 0.5 to 0.9 μm. In an even further example, the small particles can have a mean particle size of about 0.7 μm.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as acknowledgment or any form of suggestion that they constitute valid prior art or form part of the common general knowledge in any country in the world.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Example 1: Manufacture of Aqueous Suspension Pharmaceutical Composition

### Brexanolone Suspension Manufacture for Early PK Studies

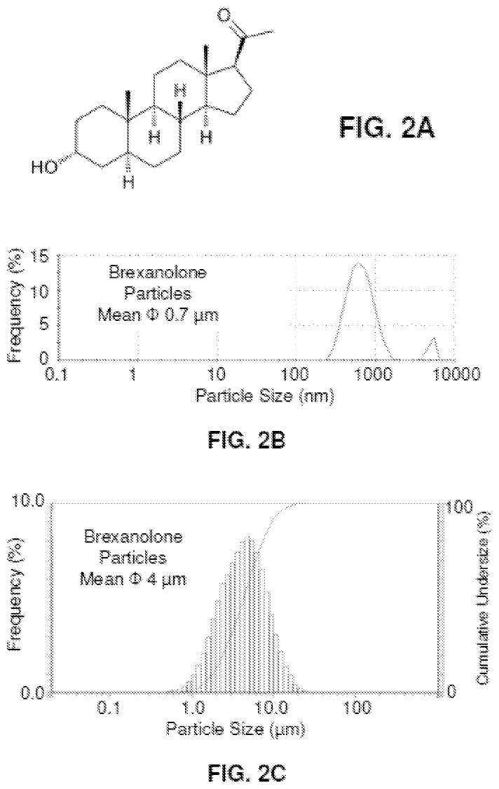

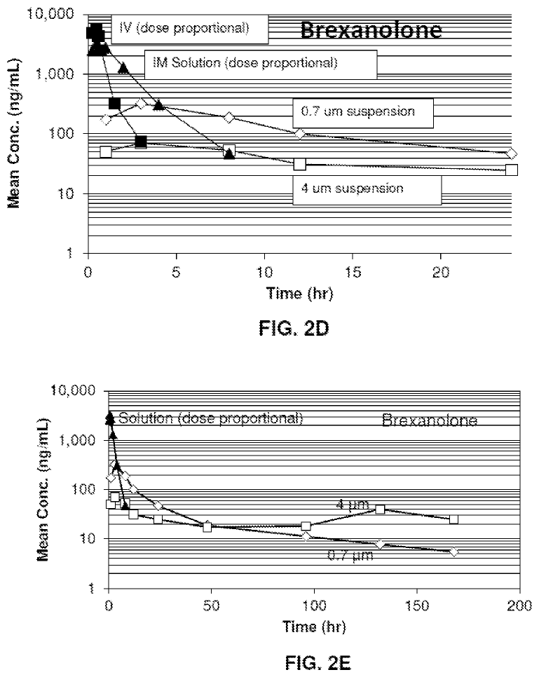

Brexanolone was purchased from a commercial vender as an active pharmaceutical ingredient (API) (FIG. 2A). The particle size of the commercial brexanolone is about 7 to 8 μm.

The commercial brexanolone was milled in the presence of water, saline, dextrose, HPMC, TWEEN 80, poloxamer 407 and glycerin with a rotation speed of about 300 to 500 rpm for about 20 to 30 minutes. The milling was performed for 1-5 cycles depending on the desired particle size. The milling media used was beads having a diameter of 0.1 to 1.0 mm.

By controlling the milling parameters, two sets of particles sizes were selected. One was small particle having a mean particle size of about 0.7 μm (FIG. 2B) and the other was large particle having a mean particle size of about 4.0 μm (FIG. 2C).

### ER Brexanolone Formulation Manufacture

The unit dose in Table 1 was prepared according to the following manufacturing process:

The manufacturing process for ER Brexanolone Suspension for Injection consists of first preparing a formulation vehicle. The formulation vehicle was prepared by dissolving polysorbate 80, polyethylene glycol 3350, mannitol, citric acid monohydrate, and sodium citrate dihydrate in Water for Injection. The solution was filtered through one sterile 0.22 micron PVDF filter discarding first 200 mL of the filtrate. The pH of formulation vehicle was checked (target pH range is 6.0-7.0) and recorded. The required amount of brexanolone drug substance was weighed and added slowly to a container with required amount of formulation vehicle while mixing slowly to wet the drug substance completely. It was then homogenized for a minimum of 10 mins at 3000 RPM to obtain a uniform suspension with no aggregates present. The pre-mix suspension was transferred to a stainless still milling chamber containing clean and depyrogenated 1 mm yttrium-stabilized zirconium oxide grinding beads of a high density and hardness and milled until target particle size is achieved. In a non-limiting example, about 560 g of 1 mm beads were loaded into 250 mL milling chamber to which about 120 g of pre-mix suspension was added. Milling was conducted at 250 RPM for about 8-10 min with periodic checking of particle size (laser light diffraction). The milled suspension was transferred to a filling container after removing beads using a screen. While mixing the suspension slowly to maintain homogeneity, milled suspension was filled into pre-sterilized glass vials followed by stoppering and crimping the overseal to secure the stopper. During filling, fill weight was checked periodically as an in-process check.

Filled vials were terminally sterilized using e-beam irradiation with an external dose range of 38-42 kGy which corresponds to an internal dose range of 26-53 kGy.

All sterilized vials were inspected for defects and foreign particulates.

TABLE 1
Brexanolone aqueous suspension composition

| Ingredient | Amount/vial (1.3 mL filled) | |
|---|---|---|
| Brexanolone | 390 | mg |
| PEG 3350 | 8.73 | mg |
| Polysorbate 80 | 8.73 | mg |
| Mannitol | 43.60 | mg |
| Sodium citrate dihydrate | 2.23 | mg |
| Citrate acid monohydrate | 0.14 | mg |

| | |
|---|---|
| Water | QS |

### Example 2: Pharmacokinetic (PK) Studies of Brexanolone Compositions of Different Particle Sizes in Rats

Suspensions of the 0.7 μm particles and the 4.0 μm particles were separately injected into rats via intramuscular (IM) injection at a dosage of 25 mg/kg of brexanolone. For comparison, comparative brexanolone solutions were injected via intramuscular (IM) injection at a dosage of 12.5 mg/kg, or intravenous (IV) injection at 1 mg/kg. Plasma brexanolone concentrations were measured at indicated time points. Data are shown in FIG. 2D-FIG. 2E. PK for the solutions were adjusted to have proportional doses.

### Example 3: Manufacture of Ganaxolone Suspensions

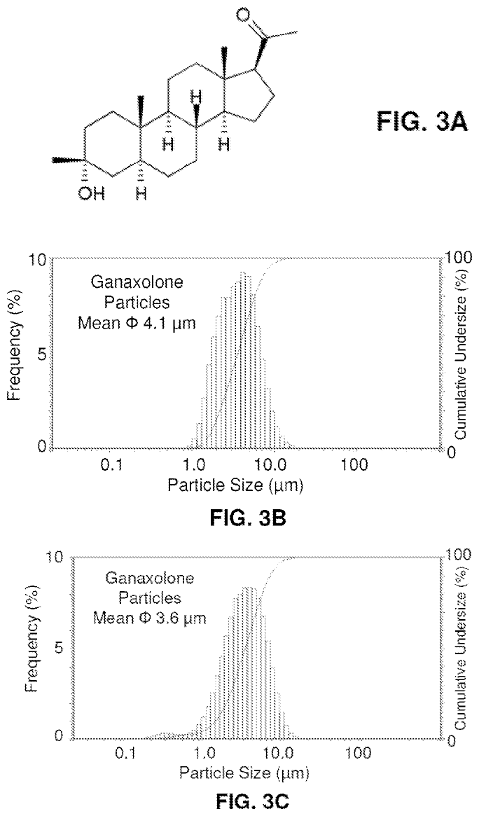

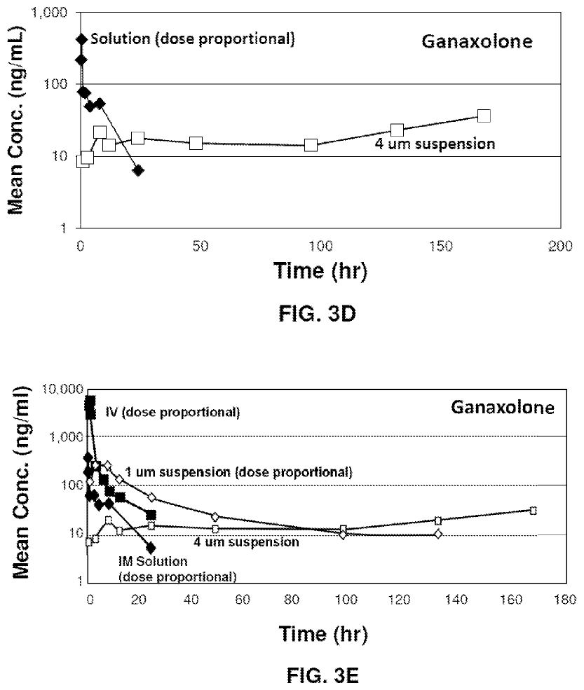

Ganaxolone was purchased from commercial vender as an active pharmaceutical ingredient (API) (FIG. 3A). The article size of the commercial brexanolone is about 47 μm.

The commercial ganaxolone was milled in the presence of water, saline, 1 mg/mL TWEEN 80 and 5 mg/mL HPMC with a rotation speed of about 200 rpm for about 20 minutes. The milling was performed for 3 cycles. The milling media used was beads having a diameter of 1.0 mm.

The milled particles had less than 1% of particles having sizes less than 1.5 μm with mean sizes of about 4.1 μm (FIG. 3B) about 3.6 μm (FIG. 3C) in two batches. Particles of having a mean particle size of about 1.0 μm were also produced.

### Example 4: Pharmacokinetic (PK) Studies of Ganaxolone Compositions of Different Particle Sizes in Rats

Suspensions of the particles having 1 μm and 4.1 μm were separately injected into rats via intramuscular (IM) injection at a dosage of 25 mg/kg of ganaxolone. For comparison, comparative ganaxolone solutions were injected via intramuscular (IM) injection at a dosage of 12.5 mg/kg, or intravenous (IV) injection at 1 mg/kg. Plasma brexanolone concentrations were measured at indicated time points. Data are shown in FIG. 3D and FIG. 3E. PK for the solutions were adjusted to have proportional doses.

### Example 5: Preparation of Brexanolone Crystalline Form

Brexanolone was purchase from commercial vender and gently grounded to form slurries. The brexanolone samples were analyzed by following analytical techniques: FT-Raman spectroscopy, FT-IR spectroscopy, Differential calorimeter (DSC), Thermogravimetric analysis (TGA-IR), Polarized light microscopy (PLM) and Powder X-Ray diffraction (PXRD). The samples were determined to be white crystalline powder consisting of irregular particles with a wide range of sizes including large brittle chunks. The DSC analysis showed a melting endotherm at 174° C. (ΔH=101 J/g). TGA analysis showed negligible (<0.1%) weight loss between 25-174° C., indicating that the brexanolone material is non-solvated.

Crystallizations were conducted in three modes: (1) temperature-cycled ripening of brexanolone slurries between 40-45° C. for two days (TC) (n=48); (2) heating the slurries to 40° C. followed by hot filtration, then storing the brexanolone solutions at 4° C. for up to two days (RC) (n=48); (3) evaporation of brexanolone solutions at ambient conditions for up to 7 days (EV) (n=48). A total of 48 solvent systems were screening for the crystal formation.

The brexanolone was crystallized from each of the solvents selected from the group consisting of dichloromethane (DCM), tetrahydrofuran (THF), ethyl acetate (EtOAc), dimethyl sulfoxide (DMSO), toluene, 2-propanol:water (9:1) (v/v), methanol (MeOH), 2-propanol (IPA), methyl t-butyl ether (MTBE), isopropyl ether (IPE), acetonitrile (MeCN), n-heptane, ethanol, water and a miscible combination thereof. All crystalline forms were analyzed using one or more of the analytical methods including PXRD, DSC, TGA and PLM.

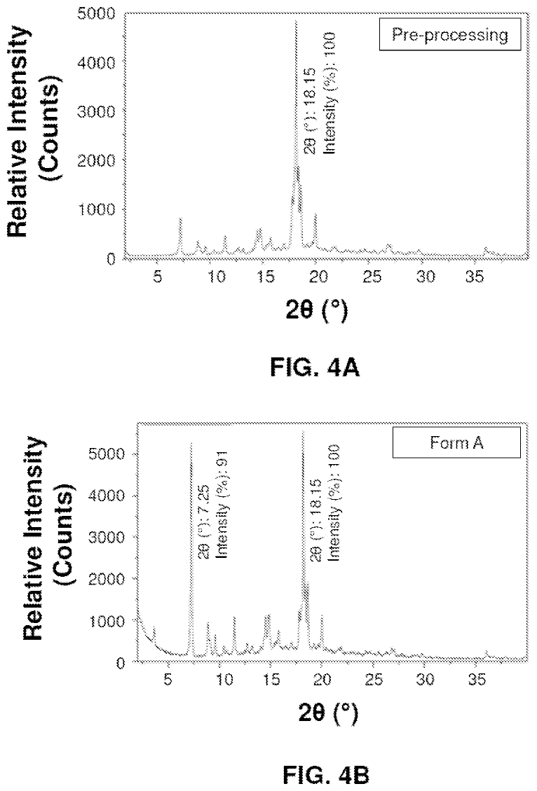

Brexanolone crystalline forms were characterized by 10 of the following peaks in Powder X-Ray Diffraction (PXRD) diffractograms, at 7.25, 8.88, 11.46, 14.50, 14.78, 17.77, 18.15, 18.32, 18.61 and 19.99±0.1 2θ(°). A comparative commercial brexanolone exhibited a single peak at 18.15 (FIG. 4A). One brexanolone crystalline form, herein referred to as “polymorph Form A” exhibited characteristic two peaks at 7.25 and 18.15 2θ(°) having relative intensities of 91 and 100, respectively (FIG. 4B), were selected for use in the pharmaceutical composition in this disclosure. Relative intensities at the peaks 18.32, 18.61, 17.77, 14.78, 19.99, 14.50, 11.46 and 8.88 were 40%, 31%, 19%, 18%, 18%, 16%, 16% and 14%, respectively. The polymorph Form A brexanolone had particle sizes in a range of 1 μm to 100 μm that can be suitable for different formulations. The polymorph Form A was showing onset melting point of 174° C. (ΔH=127 J/g). TGA analysis showed negligible (<0.1%) weight loss prior to the melting event indicating that the Form A is in a non-solvated form.

### Example 6: Single Dose Intramuscular Pharmacokinetic Assessment in Animal Models

In order to assess intramuscular (IM) bioavailability of ER Brexanolone, the PK of brexanolone after single IV administration was evaluated in multiple animal models. Following a single IV infusion in rats (1.0 mg/kg) and dogs (2.0 mg/kg) over 30 minutes, the PK parameters in Table 2 were obtained.

TABLE 2
Mean pharmacokinetic estimates in male Sprague Dawley and male Beagle dogs
following single IV infusion administration with brexanolone

| | | Dose | AUC₀₋ₜ | AUC_{0-inf} | CL | Vₛₛ | MRT_{inf} | T_{1/2} |
|---|---|---|---|---|---|---|---|---|
| Species | | (mg/kg) | (ng · h/mL) | (ng · h/mL) | (L/h/kg) | (L/kg) | (h) | (h) |
| Sprague- | Mean | 1.0 | 175 | 177 | 5.68 | 2.06 | 0.358 | 0.266 |
| Dawley rat | SD | — | 11.3 | 12.0 | 0.373 | 1.16 | 0.185 | 0.139 |
| Beagle | Mean | 2.0 | 283 | 289 | 7.22 | 2.99 | 0.441 | 1.69 |
| dog | SD | — | 62.5 | 61.8 | 0.944 | 1.66 | 0.303 | 0.619 |

Abbreviations: AUC₀₋ₜ = area under the curve from zero to the time of the last quantifiable time point; AUC_{0-inf} = area under the curve from zero to infinity; CL = clearance; Vₛₛ = volume of distribution at steady-state; MRT_{inf} = mean residence time; T_{1/2} = terminal elimination half-life; IV = intravenous; SD = standard deviation

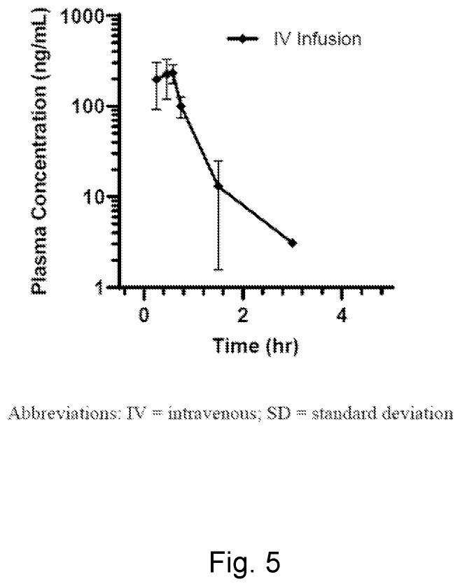

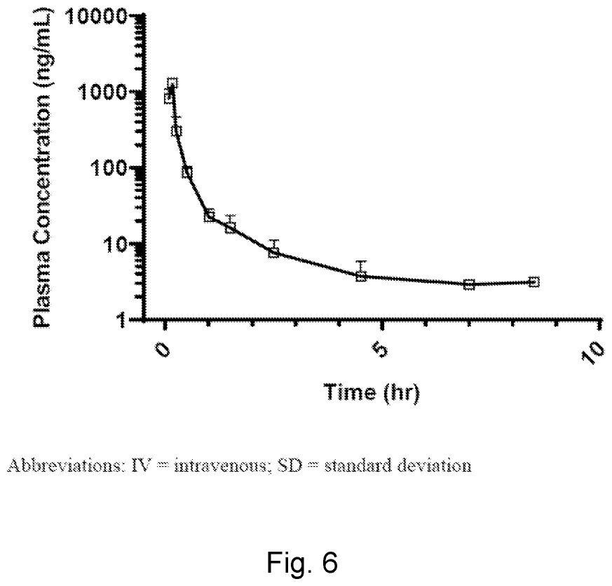

Results: The plasma concentration-time curves for IV administration are shown in FIG. 5 and FIG. 6. As shown in Table 2, following a single IV infusion dose, brexanolone CL was approximately 5.68 and 7.22 L/h/kg in rats and dogs, respectively; and it was high relative to corresponding hepatic blood flow in these two species. The Vₛₛ was approximately 2.06 and 2.99 L/kg in rats and dogs, respectively, suggesting moderate distribution of brexanolone in the body. The mean terminal elimination half-life (T_{1/2}) in rats and dogs was about 0.266 and 1.69 hours, respectively.

### IM Administration of ER Brexanolone

ER Brexanolone was administered in a single dose to rats and dogs by IM injection in a formulation of 0.6% PS80, 0.6% PEG3350, 3% mannitol, and 0.2% sodium citrate dihydrate and 0.01% citric acid monohydrate in water for injection at pH 6.0-7.0.

TABLE 3
Mean pharmacokinetic estimates in male Sprague Dawley and male Beagle dogs
following single IM administration of brexanolone suspension formulation

| | Dose | | Cₘₐₓ | Tₘₐₓ^{a} | AUC₀₋ₜ | AUC_{0-inf} | CL/F | F_{IM} |
|---|---|---|---|---|---|---|---|---|
| Species | (mg/kg) | | (ng/ml) | (h) | (ng*h/mL) | (ng*h/mL) | (L/h/kg) | (%) |
| Male | 30 | Mean | 50.4 | 3 | 5770 | 6210 | 4.9 | 109 |
| Sprague- | | SD | 7.58 | — | 1010 | 830 | 0.69 | 18.9 |
| Dawley | 60 | Mean | 88.6 | 132 | 12600 | 15200 | 4.11 | 119 |
| Rat | | SD | 27.1 | — | 1670 | 3540 | 0.935 | 15.9 |
| Male | 12 | Mean | 11.6 | 8 | 1480 | 1880 | 6.66 | 112 |
| Beagle | | SD | 6.44 | — | 413 | 414 | 1.52 | 24.8 |
| Dog | 36 | Mean | 23.6 | 0.5 | 4070 | 5020 | 7.34 | 99.7 |
| | | SD | 5.48 | — | 661 | 840 | 1.24 | 16.7 |

^{a}Tmax is presented as median, all other values are presented as mean = standard deviation (SD)
Abbreviations: Cₘₐₓ = maximun plasma concentration; Tₘₐₓ = time to maximun plasma concentration; AUC₀₋ₜ = area under the curve from zero to the time of the last quantifiable time point; AUC_{0-inf} = area under the curve from zero to infinity; CL/F = apparent clearance after IM administration; F_{IM} = bioavailability after IM administration; SD = standard deviation; IM = intramuscular

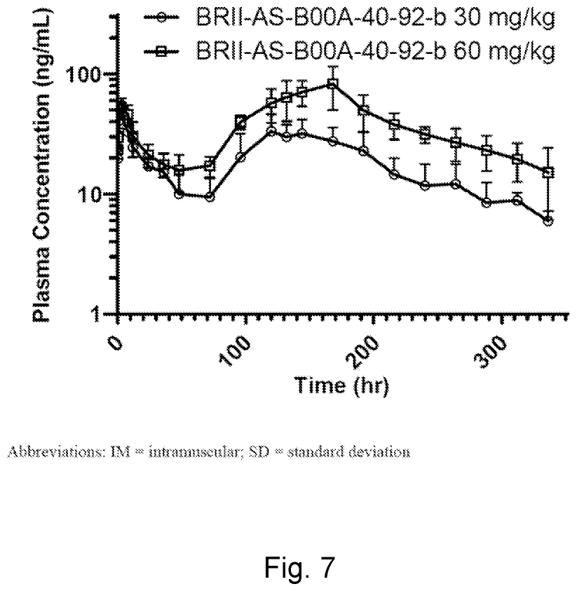

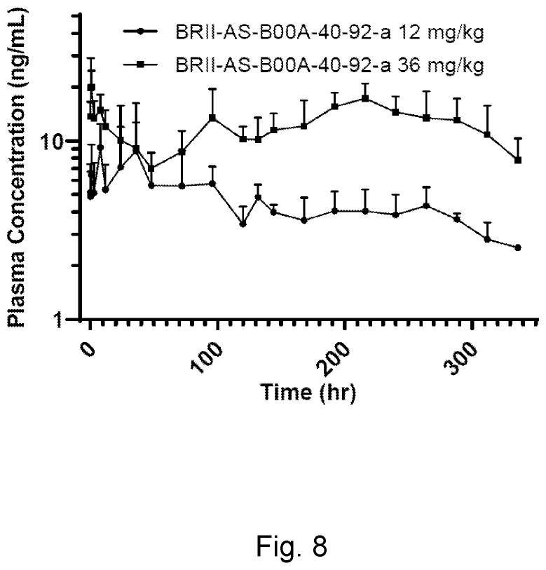

Results: In the intramuscular administration PK studies in rats and dogs, after a single IM injection of ER Brexanolone, dose dependent AUC_{0-inf} and Cₘₐₓ increases were observed in both species. In comparison with exposure from the low dose 30-minute IV infusion studies, complete absorption of brexanolone was observed in both rats and dogs at the doses tested. As evidenced by large standard deviation in Tₘₐₓ (Table 3), two-stage (fast and slow) absorption profiles were observed in both species, but the pattern is more significant in rat, likely driven by relatively slower systemic clearance (FIG. 7 and FIG. 8).

### Analytical Methods and Validation

In the rat and dog PK studies including brexanolone IV infusion and IM administrations, brexanolone was quantified in rat and dog plasma using a sensitive and specific analytical procedure. Analytes were separated by liquid chromatography (LC) and brexanolone was detected by positive mode electrospray tandem mass spectrometry (MS/MS). The lower limit of quantitation (LLOQ) was 1.0 to 3.0 ng/mL. In the GLP study, LLOQ was 1.0 ng/mL in the rat.

### Example 7: Dual Path Absorption Two Compartment Linear Population PK Model

### Overview

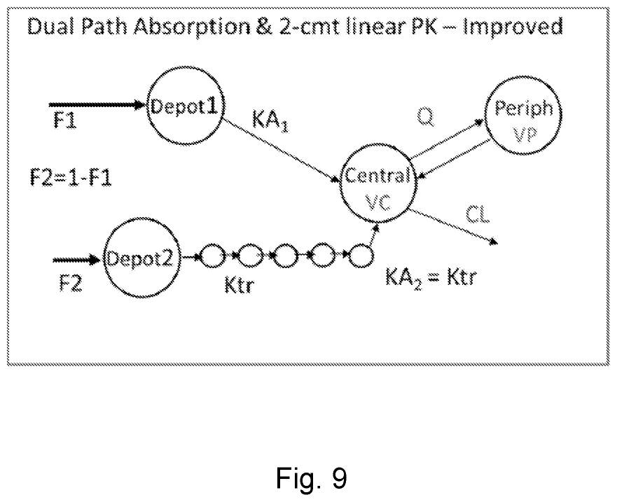

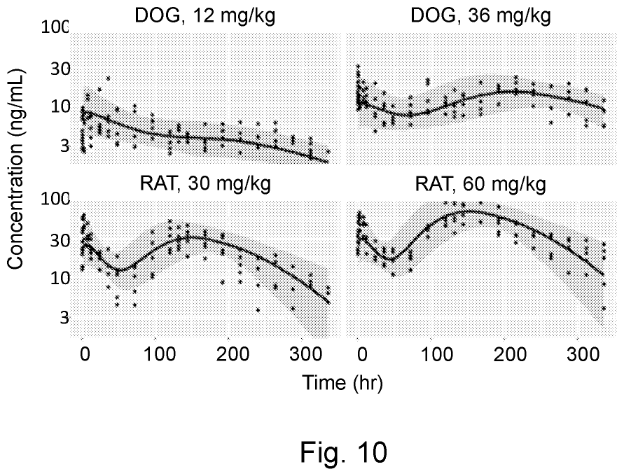

A population PK model has been developed to characterize PK profiles of brexanolone after a single IM injection of various extended-release injectable suspension formulations of brexanolone, including different particle sizes and concentrations, in rats and dogs (FIG. 9; ER Brexanolone-NC-203). In the PK model, the observed two-phase absorption processes in the rat and dog IM PK studies were characterized by two distinct paths. Described as the Path-1 in the PK model, the first absorption phase upon IM injection of the brexanolone suspension formulation was characterized by KA1 and F1. The slower and more sustained second absorption phase was described as Path-2 in the PK model using a transit compartment model. In the current model, the second in vivo absorption profiles were characterized by up to six transit compartments. As shown in FIG. 10, the observed PK profiles after single IM injection of the extended-release injectable suspension formulations in rats and dogs (see Example 1) were well characterized by the established population PK model.

### Nonclinical Study Data

Intravenous dosing of brexanolone was performed through a 30-min infusion via the jugular vein cannula (JVC) or a temporary percutaneous catheter placed into a lateral tail vein in rats, and via the cephalic vein in dogs. Different suspension formulations were also dosed intramuscularly into the rats and dogs in accordance with test facility standard operating procedures. At the designated time points, blood was collected via direct venipuncture of a cephalic or jugular vein in rats and via the cephalic or saphenous vein in dog. Blood was transferred into collection tubes containing K2EDTA anticoagulant and stored on ice until processed. Blood was processed for plasma by centrifugation. Plasma was then transferred into a 96-well container and stored in a freezer maintained at approximately −70° C. The quantitative analyses of the resulted plasma samples were conducted using a qualified LC-MS/MS method.

### Modeling & Simulation Analyses

The analyses herein was carried out according to the United States (US) Guidance for Industry: Population Pharmacokinetics, US Guidance for Industry: Exposure Response, and the European Union (EU) Guidance on Reporting the Results of Population Pharmacokinetic Analyses.

R software (version 4.0) was used to model the dissolution data. The rat and dog PK data was modeled using NONMEM (version 7.3 or greater). The RxODE package, an R package for simulating general dynamic models, and pharmacokinetic (PK) models, and pharmacokinetic-pharmacodynamic (PK-PD) models in particular, was used for simulations to derive exposure metrics. R was used for all graphical analysis of data and model outputs.

### PK Model

The overall strategy was to: 1) Start with the known 2-compartment linear PK model for brexanolone IV in human. Scale the IV model allometrically for rat and dog, then fit the model to IV rat and dog data to correct the IV PK for each species, as needed; 2) With dispersion parameters (CL, Q, VC, VP) fixed from the IV models, create a model for IM absorption of brexanolone when administered as ER Brexanolone in rat and dog by fitting to a range of IM doses (12-60 mg/kg), with various particle size Dv50 and formulation concentrations. Covariates effecting the absorption component of the model were considered in this modeling step. These were: species (rat, dog), formulation particle size Dv50 (ranged from 0.7-3.1 μm) and formulation concentration (ranged from 100-360 mg/mL); and 3) Predict brexanolone human PK profiles by retaining the known systemic PK parameters, from the brexanolone IV model, and assuming that human IM absorption is either dog-like or rat-like.

### Absorption Model

Based on early PK data from IM dosing of brexanolone using various aqueous suspension formulations in dog and rat, a two-peak PK profile was expected. Models that could characterize this two-peak profile were typically based on dual-path (fast and slow) absorption, with the slow path formulated as multiple transit compartments in the model, and the fast path as first order, as typical for extravascular absorption processes. Other dual-path options were also considered, including combinations of zero and first-order absorption.

### Human PK Model

PK model parameters for brexanolone IV in human subjects with post-partum depression, including between-subject variability, from the multi-discipline review for NDA-211317 ZULRESSO™ (brexanolone injection for intravenous use, CIV) were analyzed. The population PK model analysis based on data from the open-label study evaluating concentrations of brexanolone following administration of SAGE-547 injection in the breast milk of adult lactating women (Study 547-CLP-108) did not reveal significant difference between healthy adult women and PPD patients. Additional clinical pharmacology studies also indicted minimal gender difference in the brexanolone plasma PK profiles. As such, this model and parameters were used for prediction of brexanolone human IM PK in healthy adult subjects when administered as ER Brexanolone, with the addition of absorption-related parameters that were assumed as for either rat or dog.

### Simulations

Monte Carlo simulations of brexanolone human PK were conducted after a single IM injection of various ER Brexanolone formulations in healthy adult subjects. The predicted median and 5th-95th percentile brexanolone plasma concentration—time profile were predicted at selected dose levels for ER Brexanolone-A and ER Brexanolone-B.

Simulations were performed to evaluate time-course of brexanolone plasma exposure in human after a single IM injection of ER Brexanolone-A and ER Brexanolone-B at dose levels of 30, 100, and 300 mg. The predicted brexanolone exposures will be applied to estimate safety margins of the proposed dose levels in the phase 1 clinical study in combination with the nonclinical toxicity data.

400 subjects were simulated for each proposed dosing scenario. Model estimates of between-subject variability provided variability of simulation subjects. The median and 5th-95th percentile of the predicted brexanolone concentration time-course were computed and plotted vs time.

A total of three dose levels of 30, 100 and 300 mg from two formulations (ER Brexanolone-A and ER Brexanolone-B) have been proposed in the phase 1 clinical study in healthy adult subject: 1) 30 mg: ER Brexanolone-B; 2) 100 mg: ER Brexanolone-A and ER Brexanolone-B; and 3) 300 mg: ER Brexanolone-A and ER Brexanolone-B. Both aqueous suspension formulations have particle size Dv50 of 3 μm. The brexanolone concentration is 300 and 100 mg/mL for ER Brexanolone-A and ER Brexanolone-B, respectively. The brexanolone plasma concentrations were simulated over two-weeks post single IM injection of ER Brexanolone-A and ER Brexanolone-B at the planned dose levels.

### Results

Nonlinear mixed-effects modeling of nonclinical PK data was conducted using NONMEM (version 7.4). All other modeling, simulation and plotting were performed using R version 4.0.

Nonclinical data available for PK modeling consisted of 3 rats and 3 dogs dosed with brexanolone through intravenous administration and 36 rats and 19 dogs dosed brexanolone via single IM injection of various extended-release aqueous suspension formulations over a range of doses. The suspension formulations varied in particle size Dv50 and formulation concentration.

IV PK model in Rat and Dog

The first step in determining a PK model for brexanolone extended-release aqueous suspension formulation was to start with the currently available 2-compartment linear PK model for IV brexanolone in human scaled for rat and dog by individual animal body weights. The next step was to fit the model to rat and dog IV data with correction factors tested as needed to correct the IV PK for each species.

Rat IV model parameters did not differ significantly from the human PK parameters after accounting for body weight. The CL and Q parameters for dog required correction of 4-fold and 2.7-fold respectively to best fit the data. This is consistent with reported high systemic clearance and volume of distribution from the noncompartmental analysis of the PK data in dog. The resulting model fit well the limited rat and dog IV data.

### IM PK Model in Rat and Dog

To determine a suitable PK model to characterize brexanolone PK profiles in rat and dog after a single IM injection of various extended-release aqueous suspension formulations, the disposition parameters were fixed to those established in the IV PK model. This allowed the modeling to focus on characterization of absorption kinetic profiles post IM injection of brexanolone aqueous suspension formulations in rat and dog by fitting to available data from a range of IM doses (12-60 mg/kg), with various particle size Dv50 and formulation concentrations. Covariates considered for their effect on absorption process in this modeling step were: species (rat, dog), formulation particle size Dv50, formulation concentration and dose (mg/kg).

The final version of the model had dual path absorption with fraction F1 of drug entering the “fast path” and fraction F2=F1−1 entering the “slow path”, which was best represented by a series of 6 transit compartments. The absorption model was represented by three added parameters: F1, KA1, the fast path absorption rate constant, and MTT, the mean transit time for the slow path (where Ktr=6/MTT and Ktr is the transit rate constant for all transit compartments).

The final model demonstrated the fit of the model to the all available IM PK data in rats and dogs, and highlights the effect of particle size Dv50 and formulation brexanolone concentration on absorption kinetic profiles as well as the difference in absorption profiles between rat and dog.

Findings from the dual path absorption modeling include:
Fast path fraction F1:
   Was higher in dog as compared with what was observed in rat
   Decreased with increased dose (specified as mg/kg)
   Decreased with increased particle size Dv50
Fast path absorption rate constant KA1:
   Was slower in dog as compared with what was observed in rat
   Decreased with increased brexanolone aqueous suspension formulation concentration
Slow path absorption, as described by the mean transit time (MTT):
   Was faster in dog as compared with what was observed in rat
   No effect of particle size Dv50 or formulation concentration on MTT

To further evaluate the ability of the model to characterize the PK data from the definitive rat and dog IM PK studies, a visual predictive check was used to compare the observed data with the predicted median and 5th-95th percentile of concentration for 12 and 36 mg/kg in dog and 30 and 60 mg/kg in rat with particle size Dv50 of 3.0 μm and brexanolone formulation concentration of 200 mg/mL; and for 50 mg/kg in rat with particle size Dv50 of 3.0 μm and brexanolone formulation concentration of 100 mg/mL. Together, the predictive checks show the model can characterize the brexanolone plasma concentration-time course and variability after single IM injection of various aqueous suspension formulations in rat and dog, and over a range of dose levels and particle size Dv50.

### Example 8: Prediction of Brexanolone PK Profile in Healthy Adult Subjects

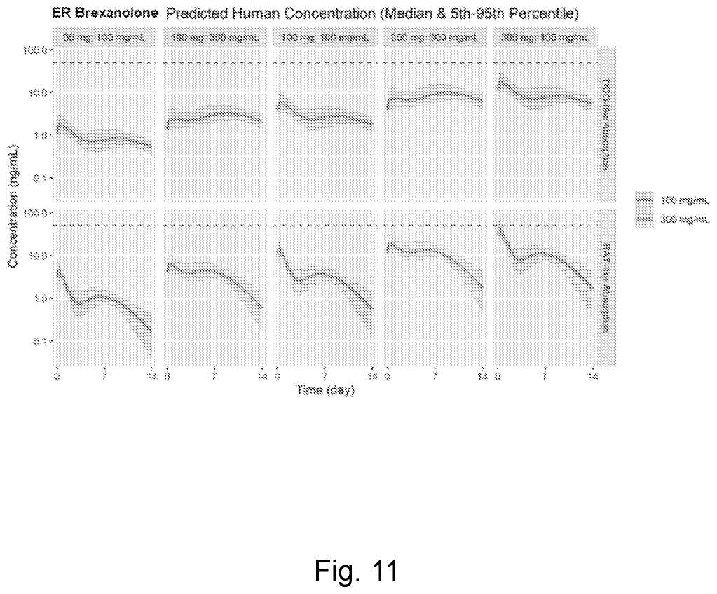

The IM absorption profiles are different between rats and dogs using various extended-release injectable aqueous suspension formulations of brexanolone, especially in terms of particle sizes and suspension concentrations (FIG. 11). In addition to the differences in the systemic clearance of brexanolone in rat and dog, species dependent physiological differences at the IM injection sites may also have impact on the absorption profiles in vivo. As such, the model parameters of the absorption kinetic profiles of different formulations in rats and dogs, including KA1, KA2 (Ktr), F1 and F2 are significantly different. In combination with the ZULRESSO™ population PK model (see FDA multi-discipline review for NDA-211317, (ZULRESSO™ [brexanolone injection for intravenous use, CIV]; US Food and Drug Administration 2018)) the IM absorption kinetic parameters from rat and dog models have been incorporated into the customized ER Brexanolone human population PK model, and subsequently applied to support the phase 1 clinical study in healthy adult subjects.

The selected ER Brexanolone clinical formulation had a particle size distribution of Dv50 of approximately 3 μm with the brexanolone concentration at 100 (ER Brexanolone-B) and 300 mg/mL (ER Brexanolone-A) for the phase 1 clinical study in healthy adult subjects. Using the established human population PK models, Monte-Carlo simulations were conducted for the planned clinical dose levels of 30, 100, and 300 mg of brexanolone administered with ER Brexanolone-A and ER Brexanolone-B, using up to 1000 virtual subjects in each simulation (FIG. 11).

Consistent with observations in the nonclinical PK studies in rat and dog, at the same dose level of 30, 100, or 300 mg, ER Brexanolone is predicted to have a faster and earlier absorption when the rat IM absorption kinetic parameters were used instead of the dog data. Similarly, at the same dose level of 100 or 300 mg, the lower formulation concentration of ER Brexanolone-B is predicted to have higher C_{max,1} and earlier T_{max,1} (Table 4).

TABLE 4
Predicted daily AUC, C_{avg}, Tₘₐₓ, and Cₘₐₓ after single intramuscular administration of ER
Brexanolone to healthy adult subjects based on dog-like or rat-like absorption kinetic profiles

| | | | Average Daily | | | | | |
|---|---|---|---|---|---|---|---|---|
| Absorption | | Formulation | AUC | C_{avg} | | | | |
| kinetic | Dose | Conc. | (ng*hr/mL)day | (ng/mL) | T_{max, 1} | C_{max, 1} | T_{max, 2} | C_{max, 2} |
| model | (mg) | (mg/mL) | (over 2-week) | (over 2-week) | (hr) | (ng/mL) | (hr) | (ng/mL) |
| Rat-like | 30 | 100 | 24.8 | 1.03 | 4 | 4.49 | 155 | 1.17 |
| | 100 | 300 | 82.2 | 3.43 | 10 | 6.29 | 143 | 4.56 |
| | 100 | 100 | 82.6 | 3.44 | 4 | 15 | 155 | 3.89 |
| | 300 | 300 | 247 | 10.3 | 10 | 18.9 | 143 | 13.7 |
| | 300 | 100 | 248 | 10.3 | 4 | 44.9 | 155 | 11.7 |
| Dog-like | 30 | 117 | 21.1 | 0.883 | 11 | 1.76 | 210.5 | 0.858 |
| | 100 | 350 | 64.9 | 2.71 | 26 | 2.47 | 214 | 3.37 |
| | 100 | 117 | 70.6 | 2.94 | 11 | 5.88 | 210.5 | 2.86 |
| | 300 | 350 | 195 | 8.12 | 26 | 7.4 | 214 | 10.1 |
| | 300 | 117 | 212 | 8.83 | 11 | 17.6 | 210.5 | 8.58 |

Abbreviations: C_{avg} = average concentration over period of time; Cₘₐₓ = maximum plasma concentration; Tₘₐₓ = time to maximum plasma concentration; AUC = area under the curve

### Example 9: Toxicology Studies

To study the toxicology of a single does IM injection, ER Brexanolone was administered to rats in a formulation of 0.6% PS80, 0.6% PEG3350, 3% mannitol, and 0.2% sodium citrate dihydrate and 0.01% citric acid monohydrate in water for injection at pH 6.0-7.0 (Table 4).

For comparison, brexanolone was administered to rats and dogs by IV infusion in a formulation of sulfobutylether-β-cyclodextrin (SBECD; Captisol®). SBECD is an excipient with known animal toxicology findings including renal tubular vacuolation and foamy macrophages in the liver and lungs of rats and dogs (Table 5).

TABLE 5
Single dose brexanolone toxicity studies

| | | | Dose Levels | | |
|---|---|---|---|---|---|
| Species | Route(s) | Duration | (mg/kg/day) | Source | |
| Single-Dose Toxicity | | | | | GLP |
| Rat | IM | Single | 30, 60 | BRII-296-NC-301 | Yes |
| | | dose | | (3174-013) | |
| Rat | IV | Single | ≤50 | Zulresso ™ | |
| | (slow bolus) | dose | | SSN-600 | |
| Dog | IV infusion | Single | ≤30 | Zulresso ™ | |
| | | dose | | SSN-599 | |

### Brexanolone IM Study

Single Dose IM Study in rat: ER Brexanolone was evaluated in a 28-day GLP single-dose toxicity study in Sprague Dawley rats with a 13-day evaluation period (Study ER Brexanolone-NC-301 [3174-013]). Groups of 10 rats/sex were administered vehicle or ER Brexanolone at 30 mg/kg (0.083 mL/kg or 0.256 mL/kg), or 60 mg/kg (0.166 mL/kg) as a single IM injection. A separate cohort of rats (3/sex VC and 9/sex for ER Brexanolone treated groups) was used for TK evaluation and received the same treatment as main study animals. Groups of 5 rats/sex were added for terminal evaluations on Day 28. Blood for TK evaluation was collected at 0.25, 0.5, 1, 3, 8, 24, 36, 48, 72, 96, 120, 144, 168, 192, 240, 288, and 336 h after the single IM injection on Day 1.

The ER Brexanolone-A and ER Brexanolone-B GLP formulations used in this toxicity study are provided in Table 6.

TABLE 6
GLP toxicity study formulation

| | Concentration (mg/mL)/w/v percentage |
|---|---|
| Composition | GLP Toxicity Study Formulation |
| Brexanolone | 117 and 362.25 mg/mL |
| PS 80 | 7.58/~0.76% |
| PEG 3350 | 7.58/~0.76% |
| Mannitol | 37.86/~3.8% |
| Sodium Citrate dihydrate | 1.94/~0.19% |
| Citric acid monohydrate | 0.123/~0.012% |

Abbreviations:
GLP = Good Laboratory Practice;
w/v = weight by volume

Observations:

ER Brexanolone-related observations of a small nodule and/or red skin discoloration on right hind limb were noted as early as 90 minutes postdose on Days 1 through Day 14 for 1 male and 1 female at 30 mg/kg (362.25 mg/mL), 5 males and 5 females at 60 mg/kg (362.25 mg/mL), and 3 males at 30 mg/kg (117 mg/mL).

ER Brexanolone-related findings at the terminal necropsy on Day 14 occurred at the injection site of males and females at ≥30 mg/kg (including both the 362.25 mg/mL and 117 mg/mL dose groups) and consisted of focal granulomatous inflammation with a central core of foreign material, associated mononuclear cell infiltration, and fibrosis. These microscopic findings correlated with clinical observations of a small nodule and/or red skin discoloration on right hind limb at 60 mg/kg (362.25 mg/mL) in 4 of 10 males and 1 of 10 females at the terminal necropsy and 1 of 5 males and 1 of 5 females at the recovery necropsy. These changes were considered partially recovered at the 60 mg/kg (362.25 mg/mL) dose level since they occurred at lower incidence and/or severity at the Day 29 recovery interval in both males and females. The changes fully recovered in both 30 mg/kg dose groups at the Day 29 recovery. All test article-related effects at the injection site were considered well tolerated due to the lack of tissue degeneration or necrosis, the localized nature of the change, and the absence of any functional change to the muscle.

Conclusions: a single intramuscular administration of ER Brexanolone to male and female rats at dose levels of 30 mg/kg and 60 mg/kg, with a 362.25 mg/mL formulation, and at 30 mg/kg, with a 117 mg/mL formulation, was well tolerated. ER Brexanolone-related findings were limited observations of a small nodule and/or red skin discoloration at all dose levels and occasionally correlated with localized injection site findings of a central core of foreign material, associated mononuclear cell infiltration, and fibrosis at terminal necropsy. These microscopic observations occurred without associated tissue degeneration/necrosis or functional change to the muscle, were similar between the groups dosed at 30 mg/kg with the 362.25 and 117 mg/mL concentrations and had partially recovered for the 60 mg/kg dose group and fully recovered for the 30 mg/kg dose group by the end of the recovery interval. Based on these findings there were no adverse effects at the local injection site for any dose level/concentration evaluated. The mean Cₘₐₓ and AUC₀₋₃₃₆ₕᵣ values for ER Brexanolone in males and females combined at 30 mg/kg (362.25 mg/mL) were 63.5 ng/mL and 2910 hr*ng/mL, respectively. The mean Cₘₐₓ and AUC₀₋₃₃₆ₕᵣ values for ER Brexanolone in males and females combined at 60 mg/kg (362.25 mg/mL) were 58.3 ng/mL and 5820 hr*ng/mL, respectively. The mean Cₘₐₓ and AUC₀₋₃₃₆ₕᵣ values for ER Brexanolone in males and females combined at 30 mg/kg (117 mg/mL) were 82.2 ng/mL and 4510 hr*ng/mL, respectively (Table 7).

TABLE 7
Toxicokinetic parameters of ER Brexanolone in male and female (combined) rat plasma following
intramuscular injection of extended release aqueous suspension formulations on Day 1

| | Dose | Dose Concentration | Cₘₐₓ | Tₘₐₓ | AUC_{0-336 hr} | | Low Dose |
|---|---|---|---|---|---|---|---|
| Group | (mg/kg) | (mg/mL) | (ng/mL) | (hr) | (hr*ng/mL) | Low Dose Ratio^{a} | Ratio^{b} |
| 6 | 30 | 362.25 | 63.5 | 0.25 | 2910 | NA | NA |
| 7 | 60 | | 58.3 | 0.5 | 5820 | NA | NA |
| 8 | 30 | 117 | 82.2 | 0.25 | 4510 | 1.29 | 1.55 |

Abbreviations: NA = not applicable; Cₘₐₓ = maximum plasma concentration; Tₘₐₓ = time to maximum plasma concentration; AUC = area under the curve
^{a}Low Dose Ratio = C_{max Group 8}/C_{max Group 6}
^{b}Low Dose Ratio = AUC_{0-336 hr Group 8}/AUC_{0-336 hr Group 6}

### Brexanolone IV Studies (Comparative Data)

Single dose IV study in rat: A single dose IV study (slow bolus) at doses ≤50 mg/kg was conducted. Rapid anesthesia (within 1 minute) was observed with a single bolus dose ≥10 mg/kg and the maximum tolerated dose (MTD) for slow bolus administration was considered to be 30 mg/kg due to shallow respiration and death of one male dosed with 50 mg/kg within 5 minutes of the bolus dose.

Single dose IV study in dog: A single dose IV study (slow bolus) at doses ≤30 mg/kg was conducted. Rapid anesthesia (within 1 minute) was observed with a single bolus dose ≥7.5 mg/kg and the MTD for slow bolus administration was considered 20 mg/kg due to irregular breathing in 1 of 4 dogs at 30 mg/kg.

Repeat dose IV study in rat: A single slow bolus IV study at 20 mg/kg followed by a continuous IV infusion at doses of 8 to 12 mg/kg/h for 6 to 12 hours a day for 3 days, a 5-day continuous IV infusion study at doses ≤240 mg/kg/day, and a 14-day continuous IV infusion study at doses ≤96 mg/kg/day were conducted in rats. The response to a continuous infusion of 8-12 mg/kg/h following an anesthetic slow bolus dose (20 mg/kg) was variable and unpredictable and an MTD could not be determined; some rats showed wakefulness while other rats showed signs of lethargy/decreased activity, unsteady gait, abnormal breathing, and increased respiration and oxygen supplementation was required for some during the infusion. The MTD for the 5-day continuous IV infusion study was 120 mg/kg/day due to signs of sedation resulting in death or premature euthanizing due to poor clinical condition at doses ≥180 mg/kg/day. Tremors and twitches were also observed at this dose. In the 14-day study, poor clinical condition (possibly due to sedation and including labored/shallow respiration, slight incoordination, decreased activity, ptosis, paleness of the whole body, and prostration) was observed in one male and one female at 96 mg/kg/day resulting in early euthanasia of this dose group on Day 11 and one male at 48 mg/kg/day. The no-observed-adverse-effect level (NOAEL) for poor clinical condition (possibly due to sedation) resulting in premature euthanasia was 18 mg/kg/day (AUC0 24 h=2910 ng*h/mL in males and 1790 ng*h/mL in females; Cmax=196 ng/mL in males and 111 ng/mL in females).

### Repeat Dose IV Study in Dog

A single slow bolus IV study at 20 mg/kg followed by a continuous IV infusion at doses of 8-48 mg/kg/h for 8 to 10 hours, and a 5-day continuous IV infusion study at doses ≤240 mg/kg/day were conducted in the dog. The response to continuous infusion of 8 to 48 mg/kg/h following an anesthetic slow bolus dose (20 mg/kg) was variable and unpredictable (some dogs remained lightly anesthetized at doses that led to edema, while other dogs could not recover from anesthesia and were euthanized; 1 dog remained deeply sedated for more than 5 hours after the end of the infusion). The MTD for the 5-day continuous IV infusion study was 60 mg/kg/day due to signs of sedation resulting in premature euthanasia at doses ≥120 mg/kg/day. Tremors and shaking were observed at 240 mg/kg/day, and one dog with tremors and shaking had a convulsion two days after the 5-day infusion was completed. The NOAEL for the 14-day continuous IV infusion study was 36 mg/kg/day due to a convulsion that occurred in one female dosed with 72 mg/kg/day approximately 7 hours after dosing stopped on Day 15 (AUC₀₋₂₄ₕ=3560 ng*h/mL in males and 3840 ng*h/mL in females; Cmax=201 ng/mL in males and 210 ng/mL in females).

### 28-Day Continuous IV Infusion Study in Rat

Brexanolone was evaluated in a 28-day GLP repeat-dose toxicity study in Sprague Dawley rats with a 28-day recovery period (Study SSN-01272). Groups of 10 rats/sex were administered vehicle (SBECD diluted in 0.9% saline [saline control, (SC)] at 3000 mg/kg/day VC high dose; 500 mg/kg/day low dose; and 1500 mg/kg/day mid-dose) or brexanolone at 10, 30, or 60 mg/kg/day as an IV continuous infusion of 24 hours/day at a rate of 2 mL/kg/h. Groups of 6 rats/sex were added as recovery animals for all dose groups except VC at 500 mg/kg/day. A separate cohort of rats (3/sex/SC and VC and 6/sex for brexanolone treated groups) was used for TK evaluation and received the same treatment as main study animals. Blood for TK evaluation (Table 8) was collected at 24, 96, 240, 384, 528, and 672 hours after the start of infusion on Day 1 and 2 and 24 hours after the end of infusion on Day 29.

### Results:

There was no test article or vehicle-related mortalities during the study. No test article related clinical observations were noted. Vehicle-related decreased activity was noted starting Day 4 and lasting for 7 days of the recovery period. Vehicle- or procedure-related limited usage of hind limbs and/or hunched posture were associated with the presence of masses and/or other procedural-related lesions (inflammation and/or bacterial sepsis) observed at the infusion site.

Increased body weight gain was observed at doses ≥10 mg/kg/day for males (126%, 126%, and 132% at 10, 30, and 60 mg/kg/day, respectively) during the first 7 days of the study and ≥30 mg/kg/day for females (160% and 193% at 30 and 60 mg/kg/day, respectively Day −1 to 7; 138% and 123% at 30 and 60 mg/kg/day, respectively Day 7 to 28) throughout the study compared to vehicle controls. Decreased body weight gain was observed at 60 mg/kg/day for males (71% at 60 mg/kg/day from Day 7 to 28) for remainder of dosing and at doses ≥10 mg/kg/day for females for the first 7 days of the recovery period. Systemic exposures were approximately dose proportional with slightly less exposures in females compared to the males (Table 6).

The NOAEL was considered 60 mg/kg/day, with Cₛₛ of 415 ng/mL and 338 ng/mL, AUC₀₋₂₄ₕ of 9960 ng*h/mL and 8110 ng*h/mL, in males and females, respectively, which was 5-times the human exposure (AUC₀₋₂₄ₕ of 1800 ng*h/mL; Cₛₛ of 74.3 ng/mL) at the MRHD of brexanolone IV of 90 μg/kg/h (Table 8).

TABLE 8
Toxicokinetic parameters from a 28-day continuous intravenous
infusion study in rats
Parameter

| 10 mg/kg/day | | 30 mg/kg/day | | 60 mg/kg/day | |
|---|---|---|---|---|---|
| M | F | M | F | M | F |

Cₘₐₓ (ng/mL)

| | | | | | |
|---|---|---|---|---|---|
| 93.8 | 63.1 | 233 | 219 | 464 | 441 |

Cₛₛ (ng/mL)

| | | | | | |
|---|---|---|---|---|---|
| 74.4 | 58.8 | 204 | 180 | 415 | 338 |

AUC_{0-24 h} (ng*h/mL)

| | | | | | |
|---|---|---|---|---|---|
| 1790 | 1410 | 4890 | 4320 | 9960 | 8110 |

Abbreviations: Cmax = maximum plasma concentration; Cₛₛ = steady state concentration; AUC_{0-24 h} = area under the curve from time zero to 24 hours post dose; F = female; M = male: AUC_{0-24 h} = AUC_{0-672 h}/28: Cₛₛ = AUC_{0-672 h}/672 h
Source: FDA multi-discipline review for NDA-211317 (Zulresso ™ [brexanolone injection for intravenous use, CIV]; US Food and Drug Administration 2018)

### 28-Day Continuous IV Infusion Study in Dog

Brexanolone was evaluated in a 28-day GLP repeat-dose toxicity study in Beagle dogs with a 28-day recovery period (Study SSN-01273). Groups of 4/sex were administered vehicle (SBECD diluted in 0.9% SC at 3600 mg/kg/day VC high dose; 600 mg/kg/day low dose; 1800 mg/kg/day mid-dose) or brexanolone at 12, 36, or 72 mg/kg/day as an IV continuous infusion of 24 hours/day at a rate of 2 mL/kg/h. Groups of 2 dogs/sex were added as recovery animals for all dose groups except the VC at 600 mg/kg/day. Blood for TK evaluation (Table 9) was collected at 24, 168, 336, 504, and 672 h after the start of infusion on Day 1.

### Results:

There was no test article- or vehicle-related mortalities during the study. Test article-related clinical signs were limited to one male dosed with 36 mg/kg which had a convulsion occurring 4 days after completing the 28-day infusion. At the end of the 28 days of continuous IV infusion at a rate of 2 mL/kg/h, the infusion rate was reduced to 1.5 mL/kg/h for 8 hours, then reduced to 1 mL/kg/h for 8 hours, then reduced to 0.5 mL/kg/h for 8 h before dosing was stopped. Increased severity of SBECD (vehicle)-related renal tubular vacuolation in males at ≥36 mg/kg and females at 72 mg/kg was observed. There were no test article-related effects and incidence and severity of SBECD (vehicle)-related findings decreased after the recovery period. Additionally, SBECD (vehicle)-related findings included clinical signs, changes in hematology and clinical chemistry parameters, increased kidney, liver, and spleen weights which were consistent with known findings for cyclodextrins.

The NOAEL was considered 12 mg/kg/day based on the convulsion after dosing completion in one 36 mg/kg dog and increased severity of SBECD-related renal tubular vacuolation at ≥36 mg/kg, Cₛₛ 69.9 ng/mL and 75.9 ng/mL, AUC₀₋₂₄ₕ 1680 ng*h/mL and 1820 ng*h/mL, in males and females, respectively which was equivalent to the human exposure (Cₛₛ 74.3 ng/mL, AUC₀₋₂₄ₕ 1800 ng*h/mL) at the MRHD of 90 μg/kg/h (Table 9).

TABLE 9
Toxicokinetic parameters from a 28-day continuous intravenous
infusion study in dogs
Parameter

| 12 mg/kg/day | | 36 mg/kg/day | | 72 mg/kg/day | |
|---|---|---|---|---|---|
| M | F | M | F | M | F |

Cₘₐₓ (ng/mL)

| | | | | | |
|---|---|---|---|---|---|
| 89.3 | 97.3 | 310 | 338 | 708 | 571 |

Cₛₛ (ng/mL)

| | | | | | |
|---|---|---|---|---|---|
| 69.9 | 75.9 | 240 | 290 | 538 | 471 |

AUC_{0-24 h} (ng · h/mL)

| | | | | | |
|---|---|---|---|---|---|
| 1680 | 1820 | 5760 | 6960 | 12900 | 11300 |

Abbreviations: Cₘₐₓ = maximum plasma concentration; Cₛₛ = steady state concentration: AUC_{0-24 h} = area under the curve from time zero to 24 hours post dose; F = female; M = male: AUC_{0-24 h} = AUC_{0-672 h}/28; Cₛₛ = AUC_{0-672 h}/672 h
Source: FDA multi-discipline review for NDA-211317 (Zulresso ™ [brexanolone injection for intravenous use, CIV]; US Food and Drug Administration 2018)

Conclusions: Compared to IV brexanolone administration, intramuscular administration of ER Brexanolone was well tolerated in animal models at multiple doses and exhibited a desirable PK profile.

### Example 10: Phase 1 Study Evaluating Safety, Tolerability, and Pharmacokinetics of ER Brexanolone in Health Subjects

### Overview

This Phase 1 open-label, safety, tolerability, and pharmacokinetic (PK) study of ER Brexanolone (brexanolone, synthetic allopregnanolone) served as a proof of concept, to evaluate if a single intramuscular (IM) injection of ER Brexanolone can achieve a brexanolone exposure profile associated with efficacy in PPD based on that of Zulresso™ (brexanolone) for intravenous (IV) use (ZULRESSO™ Prescribing Information, 2019). In the study, the ER Brexanolone extended-release aqueous suspension formulation was characterized using up to 3 different brexanolone concentrations, namely ER Brexanolone-A (300 mg/mL), ER Brexanolone-B (3-fold dilution of ER Brexanolone-A, 100 mg/mL) and ER Brexanolone-C (optional intermediate concentration) at three dose levels (30 mg, 100 mg, and up to 300 mg brexanolone), such that an optimal concentration and potential therapeutic dose could be assessed in a subsequent study in women with PPD.

### Study Design

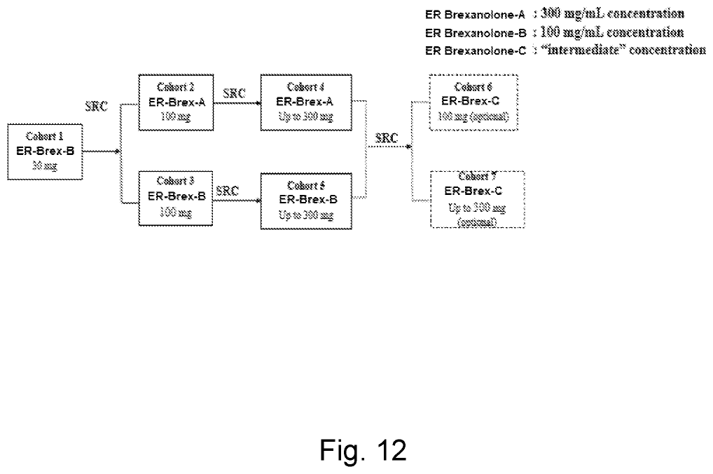

This study was a Phase 1 open-label, Single Ascending Dose (SAD) escalation study to evaluate the safety, tolerability, and PK of ER Brexanolone when administered via IM injection to healthy adult subjects. Three dose-levels (30 mg, 100 mg, and up to 300 mg) were included with two planned brexanolone concentrations (ER Brexanolone-A [higher concentration of brexanolone, 300 mg/mL] and ER Brexanolone-B [lower concentration of brexanolone, 100 mg/mL]). An additional optional intermediate concentration (ER Brexanolone-C) was also included in the study. Dose levels were evaluated in the dosing cohorts as shown in the Schema (FIG. 12). Doses may be adjusted based on the Safety Review Committee (SRC) review of the cohort safety/tolerability data. A total of five planned and two optional cohort groups were included in the study.

Typical PK parameters such as maximum plasma concentration (Cₘₐₓ), time to maximum plasma concentration (Tₘₐₓ), and apparent elimination half-life were evaluated, as well as key drug exposure characteristics such as initial drug absorption kinetics, duration of therapeutically efficacious plasma concentration, and terminal drug elimination profile. The target plasma concentration for brexanolone was chosen based upon observations of allopregnanolone levels in late pregnancy (approximately 50 ng/mL [Sage Therapeutics, Inc 2018; Kanes 2017a]), with these target levels confirmed in further study (Kanes 2017b; Meltzer-Brody 2018; Sage Therapeutics, Inc 2018) as being associated with efficacy in PPD as compared to placebo.

### Primary Objectives

To evaluate the safety and tolerability of single doses of ER Brexanolone concentrations in healthy adult subjects
To characterize the PK profiles of brexanolone after single doses of ER Brexanolone in healthy adult subjects

### Endpoints

Incidence and severity of adverse events (AEs)
Clinical assessments including vital signs, electrocardiogram (ECG) readings, laboratory test results, Sheehan Suicidality Tracking Scale (S-STS) (self-report)
PK parameters of brexanolone after single doses of ER Brexanolone include Cₘₐₓ, Tₘₐₓ, area under the curve time 0 to last measurable concentration (AUCₗₐₛₜ), area under the curve time 0 to infinity (AUC_{0-inf}), rate of absorption post IM injection (Ka), apparent terminal elimination half-life (T_{1/2}), clearance (CL), and volume of distribution (Vd).
The safety and tolerability will be assessed by clinical review of all relevant parameters including AE and laboratory assessments. All AEs will be coded with the appropriate version of the Medical Dictionary for Regulatory Activities (MedDRA) and will be tabulated according to the following:
   System organ class (SOC) and preferred term (PT)
   Severity and seriousness
   Relationship to study drug

### Subjects

Eligible subjects were assigned to each cohort group, with each cohort composed of up to 10 subjects, including at least 30% female subjects per cohort.

### Duration

Five dosing cohorts were planned and 2 dosing cohorts were optional (FIG. 12). The estimated total study participation duration for each subject was up to 6 weeks, including Screening Period (up to 4 weeks), Dosing Period (Day 1), and a postdose Follow-up Period (2 weeks). A possible Extended Follow-up Period (1 remote visit occurring after Day 14) was included, if required, based on the apparent terminal elimination half-life of ER Brexanolone.

### Dosing Levels and Concentrations

Three dose-level (30 mg, 100 mg, and up to 300 mg) cohorts were included with two planned concentrations (ER Brexanolone-A [higher concentration of brexanolone, 300 mg/mL] and ER Brexanolone-B [lower concentration of brexanolone, 100 mg/mL]). The lower concentration (ER Brexanolone-B) was prepared by on-site dilution of the 300 mg/mL concentration using a 3-fold dilution with the provided clear sterile solution containing the excipients, but not brexanolone (detailed instructions for dilution are contained in the Pharmacy Manual [PM]). An additional optional intermediate concentration (ER Brexanolone-C) was included in the study (see FIG. 12: Study Cohorts Dosing Schedule) as necessary.
Cohort 1: ER Brexanolone-B 30 mg, single IM injection at Day 1 (0.3 mL injection of 100 mg/mL dose)
Cohort 2: ER Brexanolone-A 100 mg, single IM injection at Day 1 (0.34 mL injection of 300 mg/mL dose)
Cohort 3: ER Brexanolone-B 100 mg, single IM injection at Day 1 (1 mL injection of 100 mg/mL dose)
Cohort 4: ER Brexanolone-A up to 300 mg, single IM injection at Day 1 (1 mL injection of 300 mg/mL dose)
Cohort 5: ER Brexanolone-B up to 300 mg, single IM injection at Day 1 (3 mL injection of 100 mg/mL dose)
Cohort 6: ER Brexanolone-C 100 mg, single IM injection at Day 1 (optional)
Cohort 7: ER Brexanolone-C up to 300 mg, single IM injection at Day 1 (optional)

The selection of the starting dose of 30 mg for this Phase 1 study was primarily based on animal toxicology data as well as currently available clinical results from ZULRESSO™, including maximum recommended human dose (MRHD), safety, and PK. The human equivalent doses (HED) based safety margins were calculated using the no-observed-adverse-effect level (NOAEL) from the Good Laboratory Practice (GLP) rat single IM dose study, as well as other toxicity studies summarized from the ZULRESSO™ program results (US Food and Drug Administration, Center for Drug Evaluation and Research 2018. Multi-disciplinary review and evaluation, NDA 211371, ZULRESSO™ [brexanolone]). At the clinical starting dose of 30 mg in healthy adult subjects enrolled in the current study, the safety margin was 24-fold based on the ER Brexanolone GLP rat toxicity study, and safety margins are in the range of 6- to 24-fold based on GLP toxicity studies through IV administration. The safety margin based on the ER Brexanolone rat toxicity study was 2.4-fold at the planned highest dose of 300 mg. The safety margins were also assessed based on brexanolone plasma exposures (Cmax and AUC) from the toxicity studies. In addition, the starting dose represented approximately 1/10th of the 300 mg cumulative dose from ZULRESSO™ MRHD. ER Brexanolone formulation at 300 mg/mL and 3-fold dilution to 100 mg/mL was also predicted to have a lower Cmax and lower or comparable AUC at the same dose level of ZULRESSO™. As such, the safety margin assessments, along with the known endogenous levels in pregnant women (Sage Therapeutics, Inc 2018; Kanes 2017a; Kanes 2017b; Meltzer-Brody 2018), and currently available clinical safety information, supported clinical studies of ER Brexanolone at the planned doses in healthy adult subjects.

Dosing period (Day 1): Dosing was inpatient to enable safety, tolerability and PK assessments. Eligible subjects were enrolled to receive ER Brexanolone-A (100 mg or up to 300 mg), ER Brexanolone-B (30 mg, 100 mg, or up to 300 mg), or ER Brexanolone-C (optional) (100 mg or up to 300 mg) according to the dosing schedule. Safety assessments performed on Day 1 included vital signs, pulse oximetry, and review of AEs and concomitant medication. PK blood samples were collected, and an S-STS (self-report) assessment were done predose.

Postdose follow-up period (Day 2 to Day 14): Subjects remained at the clinical investigational site for in-person assessments for 14 days. The following assessments were done during this period: brief/full physical examination (including vitals), pulse oximetry, 12-lead ECG, laboratory tests, urinalysis, pregnancy test for WOCBP, review of AEs and concomitant medication, as well as PK blood sample collection. S-STS (self-report) was done during this period. The subjects were released at the end of the inpatient period.

Extended follow-up period (1 remote visit occurring after Day 14): The possible Extended Follow-up remote visit occurred after Day 14, if required, based on the apparent terminal elimination half-life of ER Brexanolone. An AE assessment was conducted during this remote visit.

### Inclusion Criteria

Subjects were eligible to be included in the study only if all of the following criteria apply:
Age—must be 18 (or age of legal consent, whichever is older) to 50 years of age inclusive, at the time of signing the informed consent
Type of Subject and Disease Characteristics
   a. Healthy as determined by medical evaluation including medical history, physical examination, laboratory tests, and cardiac monitoring using 12-lead ECG
   b. Has venous access sufficient to allow for blood sampling as per the protocol
Weight—as a body mass index (BMI) within the range of 18.0-35 kg/m2 (inclusive), and, in the opinion of the investigator, the subject's body habitus would not preclude the ability to correctly inject intramuscularly at the site of injection.
Sex—male and female
Contraceptive use by men or women should be consistent with local regulations regarding the methods of contraception for those participating in clinical studies.
Male subjects: male subjects with female partners of childbearing potential agreed to meet 1 of the following contraception requirements from the time of study drug administration through the end of the study:
   a. Total abstinence, or
   b. Vasectomy with documentation (subject's self-report) of azoospermia, or
   c. Barrier form of contraception (condom) with spermicide, or
   d. Female partner user of an intrauterine device or hormonal contraceptives including oral, implantable, injectable or transdermal contraceptives within 12 weeks prior to male signing the informed consent form (ICF).
Male subjects also agreed not to donate sperm until 90 days after dosing.
Female subjects: A female subject was eligible to participate if she was:
   a. of non-childbearing potential, defined as a premenarchal, or pre-menopausal with documented (subject's self-report) hysterectomy, or bilateral tubal ligation or oophorectomy,
   b. of childbearing potential, not breastfeeding and agreed to use one of the following acceptable methods of contraception through the end of the study:
      total abstinence, or
      barrier form of contraception such as condom or occlusive cap with spermicide, or
      an intrauterine device, or hormonal contraceptives including oral implantable, injectable or transdermal contraceptives initiated at least 12 weeks prior to signing the ICF. Women of childbearing potential (WOCBP) must have a negative serum pregnancy test at screening and a negative urine pregnancy test on Day −1 prior to study drug administration.
Informed Consent—Capable of giving signed informed consent, which included compliance with the requirements and restrictions listed in the ICF and in the protocol
Other Inclusion Criteria
   a. Agreed to not donate blood during the duration of the study
   b. Agreed not to increase physical activity for 2 weeks after study drug administration

Exclusion Criteria

Subjects were excluded from the study if any of the following criteria apply:
Medical Conditions
   a. Significant history or clinical manifestation of any metabolic, allergic, dermatological, hepatic, renal, hematological, pulmonary, infectious, cardiovascular, gastrointestinal, neoplastic (with the exception of basal or squamous cell cancer), neurological, or psychiatric disorder (as determined by the Investigator) capable of significantly altering the absorption of drugs; of constituting a risk when taking the study medication; or of interfering with the interpretation of the data.
   b. Any major surgical procedure or hospitalization within 6 months prior to Day 1 or during the study, unless deemed not clinically significant by the Investigator.
   c. A history of significant hypersensitivity, intolerance, or allergy to any drug compound, food, or other substance, unless approved by the Investigator.
   d. History or presence of an abnormal ECG which, in the Investigator's opinion, is clinically significant. A QTc interval duration (either Bazett or Fridericia)>450 ms obtained as an average from triplicate screening ECGs after at least 10 minutes at rest.
   e. History of alcohol or other substance use disorders within 1 year prior to screening according to the American Psychiatric Association Diagnostic and Statistical Manual of Mental Disorders (5th edition) criteria, or recent use of drugs of abuse or a positive urine screen for drugs of abuse at screening.
   f. History of depression or suicidal thoughts and/or behaviors within 1 year prior to screening.
   g. History of intolerance to IM injection.
   h. History of hepatic decompensation, including ascites, hepatic encephalopathy and/or esophageal or gastric varices.
   i. SARS-CoV-2 exposure history or clinical history of COVID-19, including positive COVID-19 RNA test result on Day −1 (test administered on Day −2).
   j. Received any vaccine within 14 days prior to Day 1 or plans to receive a vaccine any time during the study.
Prior/Concomitant Therapy
   k. Use prescription drugs within 14 days before Day 1 and throughout the study except for a stable dose of: 1) prescription medications to treat pre-existing medical conditions such as gastrointestinal reflux, asthma, allergy, hypercholesterolemia, and hypertension. Hypertension must be well controlled on one medication for >6 months. Asthma must be well controlled, requiring, on average, use of a rescue bronchodilator no more than twice per week. Hormone replacement therapy and oral, injectable, subdermal, intravaginal, or implantable contraceptives, as well as intrauterine device, and intrauterine hormone-releasing system, are permitted for contraception. Inhaled and topical steroids are permitted.
   l. Use of antiplatelet, anticoagulant, or antiepileptic medications within 30 days prior to Day 1 and throughout the study.
   m. Use of antidepressants, opioids, or central nervous system acting drugs, such as benzodiazepines, within 14 days before Day 1 and throughout the study.
   n. Use of over the counter (OTC) medication or herbal remedy, e.g., Traditional Chinese Medicine, within 14 days before Day 1 and throughout the study, with the following exceptions of permitted OTC medications: paracetamol (acetaminophen)≤2 g/day, aspirin ≤3 g/day or ibuprofen <1.2 g/day; or topicals.
   o. Consumption of foods or juices containing cranberries, pineapples, Seville oranges, grapefruit, or caffeine (xanthine-containing products) within 72 hr prior to Day 1 and throughout the study, unless deemed acceptable by the Investigator.
   p. Consumption of herbal tea, energy drinks, herbal products (e.g, St. John's wort, milk thistle), or supplement supra-therapeutic doses of vitamins within 14 days prior to Day 1 and throughout the study, with the exception of those approved by the Investigator, Medical Monitor, and/or Sponsor.
Prior/Concurrent Clinical Study Experience
   q. Received an investigational monoclonal antibody within 90 days, or other investigational agent within 90 days or 5 half-lives (whichever is longer), before study drug administration, or are active in the follow-up phase of another clinical study involving interventional treatment. Subjects also agreed not to take part in any other study at any time during their participation in this study, inclusive of the follow-up period.
Diagnostic assessments
   r. Systolic blood pressure >140 mmHg or a diastolic blood pressure of >90 mmHg after approximately 10 minutes resting at screening.
   s. Calculated glomerular filtration rate of <60 mL/min/1.73 m2 by estimated glomerular filtration rate (eGFR) using standardized local clinical methodology.
Other Exclusion Criteria
   t. Has an average weekly alcohol intake that exceeds 21 units of alcohol per week (males up to age 50) or 14 units per week (males >50 yr and females) within 30 days prior to screening. One unit: 1 glass of wine 5 oz or 150 mL; 12 oz or 360 mL of beer; 1.5 oz or 45 mL of distilled spirits.
   u. Are unwilling to stop alcohol consumption within 72 hr prior to Day 1 and for the duration of the study (as confirmed by alcohol breath screen).
   v. Use any tobacco- or nicotine-containing products including, but not limited to cigarettes, electronic cigarettes (of any kind), pipes, cigars, chewing tobacco, nicotine patches, nicotine lozenges, or nicotine gum within 6 months prior to Day 1 and during the study.
   w. Need special dietary restrictions, unless the restrictions are approved by the Investigator, Medical Monitor, and/or Sponsor.
   x. Any conditions which, in the opinion of the Investigator, would make the subject unsuitable for enrollment or could interfere with the subject's participation in or completion of the study.
   y. Donated more than 500 mL of blood within 90 days before study drug administration.

### Study Drug

ER Brexanolone was supplied as a sterile aqueous suspension formulation for IM injection at a final concentration of 300 mg/mL (Table 10). It was supplied in a single-use vial shipped to the site at 2-8° C. and maintained at the site at 2-8° C. ER Brexanolone vials were kept in the original box to protect the vials from light. Each vial contained 1.3 mL of suspension (brexanolone 300 mg/mL) in a 2-mL clear brown glass vial. A clear sterile solution containing the excipients, but not brexanolone, was provided for dilution (2.2 mL per vial) in a clear brown glass vial.

TABLE 10
Phase 1 Doses of ER Brexanolone for IM Study

| | | | |
|---|---|---|---|
| Type | Drug | Drug | Drug |
| Dose | Aqueous suspension | Aqueous suspension | Aqueous suspension |
| Formulation | | | |
| Unit Dose | 300 mg/mL | 100 mg/mL | To be Determined |
| Strength(s) | | | (if required) |
| Dosage Level(s) | Single dose of 100 mg | Single dose of 30 mg, | Single dose of 100 mg |
| | and up to 300 mg | 100 mg and up to 300 mg | and up to 300 mg |
| Route of | IM injection | IM injection | IM injection |
| Administration | | | |

### Safety Assessments

Safety-related assessments included physical examinations, 12-lead ECGs, vital signs, AEs, pulse oximetry, clinical laboratory tests (Table 11) and S-STS (self-report). These assessments were performed at screening and at specific times during the study (during inpatient observation periods). An AE assessment was conducted during the Extended Follow-up Period remote visit, if this visit was required. During any period of the study, if an adverse event of special interest (AESI), including excessive sedation/somnolence, LOC, or syncope/presyncope was reported, detailed clinical observational data was collected, and unscheduled PK sampling performed. Injection-site reactions (ISRs) were also identified as AESIs in this study.
Physical Examinations—Cardiovascular, Respiratory, Gastrointestinal and Neurological systems. Height, body weight and BMI will also be measured and recorded at the Screening visit.
Vital Signs—Blood pressure, pulse rate, respiratory rate and temperature were assessed at the Screening visit and Day −1 through Day 8, Day 10, Day 12, Day 14, and Early Discontinuation.
Pulse Oximetry—Pulse oximetry were conducted continuously on Day 1
Electrocardiograms—Triplicate 12-lead ECGs were obtained using an ECG machine that automatically calculates the heart rate and measures PR, QRS, QT, and QTc intervals (Day 1, Day 14).
Clinical Safety Laboratory Assessments (see Table 11)

TABLE 11
Clinical laboratory assessments
Hematology

| | |
|---|---|
| WBC | RBC |
| Hemoglobin | Platelets |
| Percentage of neutrophils | Neutrophils count |
| Percentage of lymphocytes | Lymphocytes count |
| Percentage of monocytes | Monocytes count |
| Percentage of eosinophils | Eosinophils count |
| Percentage of basophils | Basophil cell count |

Blood Chemistry

| | |
|---|---|
| Albumin | Gamma glutamyl transferase (GGT) |
| Calcium | Glucose (fasting) |
| Carbon dioxide/bicarbonate | Lactate dehydrogenase (LDH) |
| Chloride | Potassium |
| Creatine kinase | Sodium |
| Triglycerides | Uric acid |
| Globulin | Total cholesterol |
| Low density lipoprotein | High density lipoprotein |
| cholesterol (LDL-C) | cholesterol (HDL-C) |

Liver and Kidney Function Tests

| | |
|---|---|
| Alkaline phosphatase (ALP) | Creatinine |
| Aspartate Aminotransferase (AST) | Estimated glomerular filtration |
| | rate (eGFR) |
| Alanine Aminotransferase (ALT) | Urea |
| Bilirubin (total and direct) | |

Urinalysis

| | |
|---|---|
| Bilirubin | Protein |
| Glucose | Red blood cells (RBC) |
| Ketone bodies | pH |
| White blood cells (WBC) | Specific gravity |
| Microscopic examination (if | Urobilinogen |
| there is clinical indication) | |
| Nitrite | Urine occult blood |

Pregnancy Testing

| | |
|---|---|
| Serum pregnancy tests | Urine pregnancy tests |

Other Tests

| | |
|---|---|
| Viral serology to include HIV | Urine drug screen to include: |
| antibody, hepatitis B surface | amphetamines, barbiturates, |
| antigen [HBsAg], and hepatitis C | benzodiazepines, MDMA (ecstasy), |
| virus antibody | cocaine, methadone, |
| SARS-CoV-2 nucleic acid test | methamphetamine, opiates, |
| | oxycodone, PCP (angel dust), |
| | tetrahydrocannabinol (THC), and |
| | tri-cyclic antidepressants (TCA) |
| | Alcohol breath screen |

### Adverse Events

Adverse events (AEs) were reported by the subject.

The Investigator and any qualified designees were responsible for detecting, documenting, and recording events that meet the definition of an AE or SAE and remain responsible for following up on AEs that are serious, considered related to the study drug or study procedures, or that caused the subject to discontinue the study.

### Pharmacokinetics

Whole blood samples were collected for measurement of plasma concentrations of ER Brexanolone.
Additional samples were collected at other time points during the study if warranted and agreed upon between the Investigator and the Sponsor. The timing of sampling, was altered during the course of the study based on newly available data (e.g., to obtain data closer to the time of peak plasma concentrations) to ensure appropriate monitoring.
Instructions for the collection and handling of biological samples were provided by the Sponsor. The actual date and time (24-hour clock time) of each sample were recorded.
Samples were used to evaluate the PK of ER Brexanolone (Table 12). Details regarding the processing, shipping, and analysis of the samples were provided in the Pharmacokinetics Manual. Samples collected for analyses of ER Brexanolone plasma concentration were also used to evaluate safety aspects related to concerns arising during or after the study.
At visits during which whole blood samples for the determination of multiple aspects of ER Brexanolone were taken, e.g., laboratory and PK, one sample of sufficient volume was used.

TABLE 12
Pharmacokinetic Assessment Timepoints

| Study Day/Week | | Protocol Time | PK Blood Sample |
|---|---|---|---|
| Week 0 | Screening | Day −1 | X |
| Week 1 | Day 1 | Predose (0 hr) | X^{a} |
| | | 0.5 hr ± 5 min | X |
| | | 1 hr ± 5 min | X |
| | | 3 hr ± 5 min | X |
| | | 8 hr ± 5 min | X |
| | | 12 hr ± 5 min | X |
| | Day 2 | 24 hr ± 30 min | X |
| | | 36 hr ± 30 min | X |
| | Day 3 | 48 hr ± 30 min | X |
| | Day 4 | 72 hr ± 30 min | X |
| | Day 5 | 96 hr ± 30 min | X |
| | Day 6 | 120 hr ± 30 min | X |
| | Day 7 | 144 hr ± 30 min | X |
| Week 2 | Day 8 | 168 hr ± 30 min | X |
| | Day 10 | 216 hr ± 30 min | X |
| | Day 12 | 264 hr ± 30 min | X |
| | Day 14 | 336 hr ± 30 min | X |

^{a}At ≤15 minutes prior to dosing

### Results

PK Analysis—Cohort 4:

Subjects in Cohort 4 were administered 1 mL of a 300 mg/mL dose of ER Brexanolone by intramuscular injection. A PK analysis of four of the subjects in Cohort 4 (2 female and 2 male subjects) is provided in Table 13.

TABLE 13
PK parameters for IM administration of ER Brexanolone obtained from subjects of Cohort 4

| | Cₘₐₓ | *Tₘₐₓ | AUC_{0-312 h} | AUC_{0-inf} | Base Line C_{-24 h} | C_{312 h} | *t_{1/2} |
|---|---|---|---|---|---|---|---|
| Subject ID | (ng/mL) | (h) | (ng*h/mL) | (ng*h/mL) | (ng/mL) | (ng/mL) | (h) |
| N = | 4 | 4 | 4 | 2 | 1 | 4 | 2 |
| Cohort 4 | 22.4 ± 5.44 | 252 | 3540 ± 916 | 3600 | 1.03 ± NC | 14.5 ± 10.7 | 60 |
| (300 mg/mL) | [24%] | [216-312] | [26%] | | [101%] | [74%] | [37-82] |

*Median and range reported for time related PK parameters; t_{1/2} and AUC_{0-inf} not reported if % extrapolated AUC > 30%

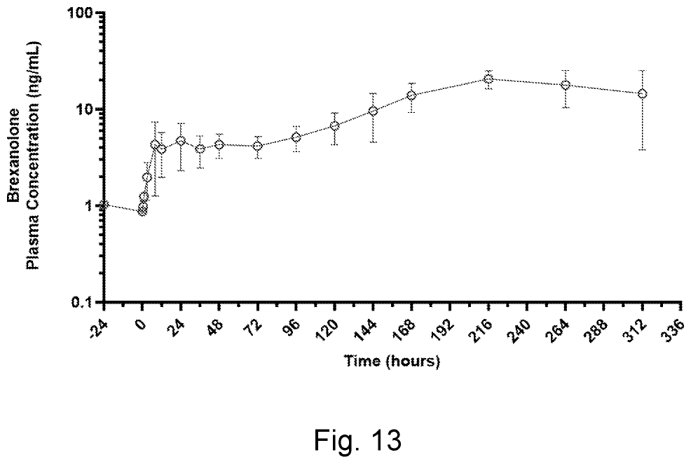

According to FIG. 13, the plasma concentration of brexanolone increased slightly up to 216 h and then is maintained at a consistent level for the duration of the study.

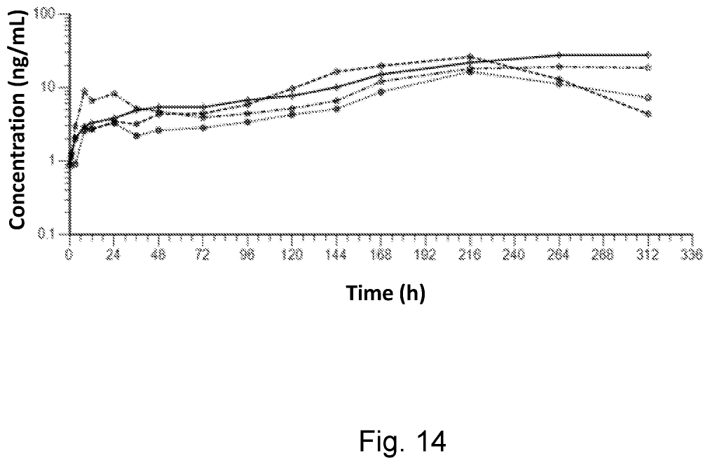

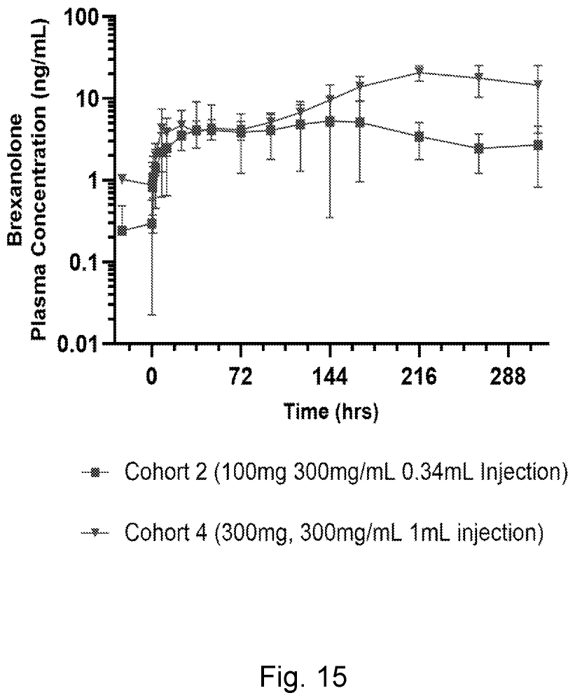

As shown by the overlaid data in FIG. 14, inter-subject variability was low (CV<30%) based on observed values for Cₘₐₓ and AUC₀₋₃₁₂ₕ. The results in FIG. 15 for three ascending doses show proportionality as there was roughly linear exposure increase (AUC₀₋₃₁₂ₕ) in the range from 30 to 300 mg. On Day 14, mean plasma concentration was ˜14-fold higher than the mean pre-dose baseline level. Furthermore, there was no drug-dumping observed in any subject.

PK Analysis—Cohort 5:

Subjects in Cohort 5 (2 female and 2 male subjects) were administered 3 mL of a 100 mg/mL (300 mg total) dose of ER Brexanolone by intramuscular injection. A PK analysis of three of the subjects in Cohort 5 is provided in Table 14.

TABLE 14
PK parameters for IM administration of ER Brexanolone obtained from subjects of Cohort 5

| | Cₘₐₓ | *Tₘₐₓ | AUC_{0-312 h} | AUC_{0-inf} | Base Line C_{-24 h} | C_{312 h} | *t_{1/2} |
|---|---|---|---|---|---|---|---|
| Subject ID | (ng/ml) | (h) | (ng*h/mL) | (ng*h/mL) | (ng/mL) | (ng/ml) | (h) |
| N= | 4 | 3 | 3 | 3 | 1 | 3 | 3 |
| Cohort 5 | 26.7 ± 6.70 | 168 | 4720 ± 960 | 4840 ± 990 | 0.297 ± NC | 2.58 ± 1.09 | 33 |
| (100 mg/mL) | [24%] | [168-216] | [20%] | [20%] | | [42%] | [30-33] |

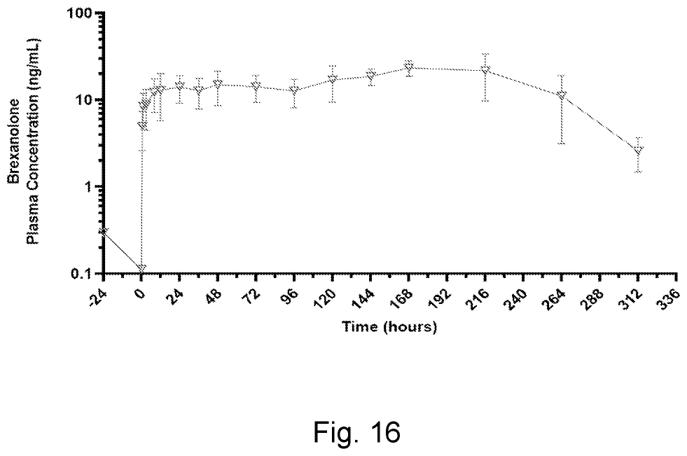

According to FIG. 16, the plasma concentration of brexanolone was maintained at a consistent level for about 264 h before tapering off slightly.

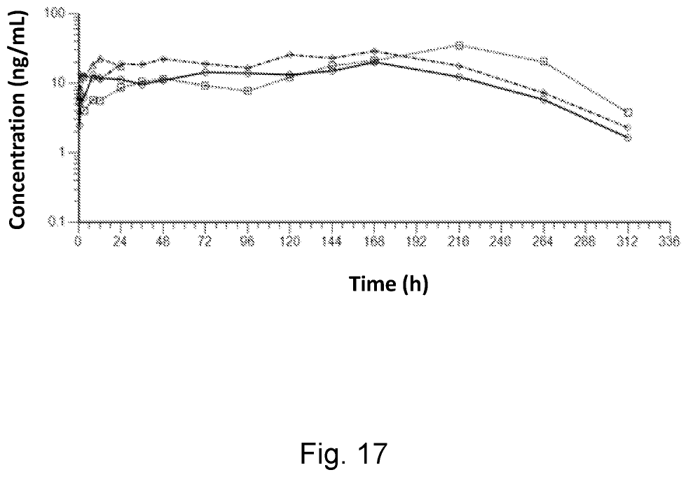

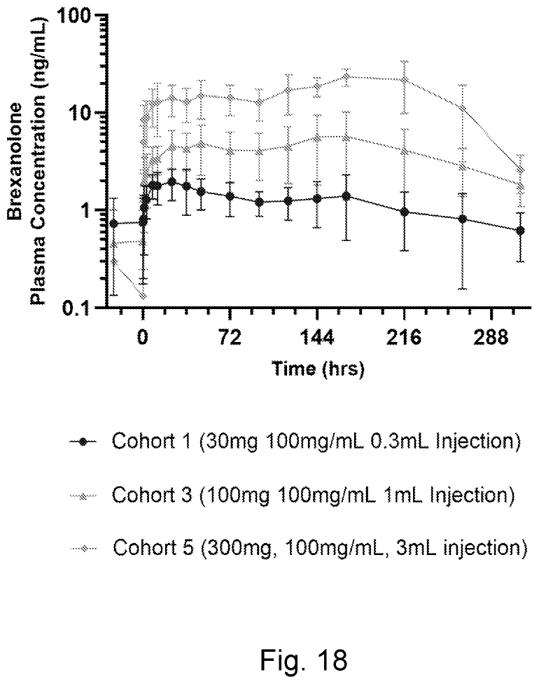

As shown by the overlaid data in FIG. 17, inter-subject variability was low (CV<30%) based on observed values for Cₘₐₓ and AUC₀₋₃₁₂ₕ. The results in FIG. 18 for three ascending doses show proportionality as there was roughly linear exposure increase (AUC₀₋₃₁₂ₕ) in the range from 30 to 300 mg. On Day 14, mean plasma concentration was ˜9-fold higher than the mean pre-dose baseline level. Furthermore, there was no drug-dumping observed in any subject.

### Example 11: Phase 2 Clinical Trial Evaluating ER Brexanolone for the Prevention of Postpartum Depression in Female Participants

A phase 2 randomized, double-blind, placebo-controlled, study to evaluate the safety, tolerability, and efficacy of ER Brexanolone in the prevention of postpartum depression (PPD) in adult women at risk of developing PPD.

### Study Design

This is a phase 2 randomized, double-blind, placebo-controlled trial of ER Brexanolone administered via IM injection to adult women at risk of PPD. The study is designed to evaluate the safety, tolerability, and efficacy of single doses of ER Brexanolone.

Eligible subjects will be randomized in the clinical trial phase immediately postpartum and administered ER Brexanolone or placebo as a single dose within 24-48 hours after delivery.

Subjects with an episode of PPD will be defined as follows: Subjects with a Hamilton Depression Rating Scale (HAM-D)₁₇ total score of 15 or higher on two occasions 1 week apart will be evaluated by a blinded psychiatrist to confirm the presence of DSM-5 criteria major depression (PPD).

Treatment groups: ER Brexanolone or placebo

### Objective

To evaluate the safety and tolerability of a single dose of ER Brexanolone administered via intramuscular injection (IM) in adult women at risk of developing PPD.

To evaluate the efficacy of a single dose of ER Brexanolone administered IM in adult women at risk of developing PPD.

### Patient Population

This trial will enroll Nondepressed adult pregnant women at risk of developing PPD.

Number of Participants: The clinical trial is planned to include up to 50 subjects total; n=25 per arm, randomized 1:1 ER Brexanolone vs placebo.

Inclusion Criteria: Female subjects must meet the following criteria to be eligible to participate in this clinical trial:
Must be 18 (or age of legal consent, whichever is older) to 45 years inclusive, at the time of the signing informed consent.
Capable of giving signed informed consent as described in the protocol which includes compliance with the requirements and restrictions listed in the informed consent and in the protocol.
Nondepressed (HAM-D₁₇≤7) pregnant women (gestation of 35 weeks or less) with at least 1 past episode of postpartum onset major depression requiring medical intervention within 5 years prior to screening.
Healthy as determined by medical evaluation including medical history, physical examination, cardiac monitoring using 12-lead ECG, and laboratory tests.
Have venous access sufficient to allow for blood sampling as per the protocol.
Agree to not donate blood during the duration of the study.
Have a body mass index (BMI) within the range of ≥18.0 kg/m² and ≤35 kg/m², and, in the opinion of the investigator, the subject's body habitus would not preclude the ability to correctly inject intramuscularly at the site of injection.

Exclusion Criteria: A female subject who meets any of the following criteria at (unless otherwise specified) will be excluded from this clinical trial:
Significant history or clinical manifestation of any metabolic, allergic, dermatological, hepatic, renal, hematological, pulmonary, infectious, cardiovascular, gastrointestinal, neoplastic (except for basal or squamous cell cancer), neurological, or psychiatric disorder (as determined by the Investigator) capable of significantly altering the absorption of drugs; of constituting a risk when taking the study medication; or of interfering with the interpretation of the data.
Met DSM-5 criteria for any other axis I diagnosis (except generalized anxiety or panic disorder) or for antisocial or borderline personality disorder and those with psychosis or bipolar disorder
Any major surgical procedure or hospitalization within 6 months prior to Day 1 or during the study, unless deemed not clinically significant by the Investigator.
A history of significant hypersensitivity, intolerance, or allergy to any drug compound, food, or other substance, unless approved by the Investigator
History or presence of an abnormal ECG which, in the Investigator's opinion, is clinically significant. A QTc interval duration (either Bazett or Fridericia)>450 ms obtained as an average from triplicate screening ECGs after at least 10 minutes resting at screening.
Systolic blood pressure >140 mmHg or a diastolic blood pressure of >90 mmHg after approximately 10 minutes resting at screening.
Calculated glomerular filtration rate of <60 mL/min/1.73 m² by estimated glomerular filtration rate (eGFR) using standardized local clinical methodology.
History of alcohol or other substance disorders within 1 year prior to screening according to the American Psychiatric Association Diagnostic and Statistical Manual of Mental Disorders, 5th edition criteria, or recent use of drugs of abuse or a positive urine screen for drugs of abuse at screening.
History of depression or suicidal thoughts and/or behaviors within 1 year prior to screening.
Receive an investigational monoclonal antibody within 90 days, or other investigational agent within 90 days or 5 half-lives (whichever is longer), before study drug administration or are active in the follow-up phase of another clinical study involving interventional treatment. Subjects must also agree not to take part in any other study at any time during their participation in this study, inclusive of the follow-up period.
History of intolerance to IM injection.
Any conditions which, in the opinion of the Investigator, would make the subject unsuitable for enrollment or could interfere with the subject's participation in or completion of the study.
History of clinically significant chronic liver disease from any cause, presence of hepatitis B surface antigen, hepatitis C virus antibody or human immunodeficiency virus antibody.

Excluded Medications and Foods
Use prescription drugs within 14 days before Day 1 and throughout the study except for a stable dose of: 1) prescription medications to treat pre-existing medical conditions such as gastrointestinal reflux, asthma, allergy, hypercholesterolemia, and hypertension. Hypertension must be well controlled on 1 medication for >6 months. Asthma must be well controlled, requiring, on average, use of a rescue bronchodilator no more than twice per week. Hormone replacement therapy and oral, injectable, subdermal, intravaginal, or implantable contraceptives, as well as intrauterine device, and intrauterine hormone-releasing system, are permitted for contraception. Inhaled and topical steroids are permitted.
Use of antiplatelet, anticoagulant, or antiepileptic medications within 30 days prior to Day 1 and throughout the study.
Use of any psychotherapy or psychotropic medications after the first trimester of pregnancy.

Investigational Product: ER Brexanolone (brexanolone, synthetic allopregnanolone) is a natural neuroactive steroid (NAS) gamma-aminobutyric acid (GABA) A receptor positive modulator. ER Brexanolone is formulated as an extended-release aqueous suspension for IM use.

The ER Brexanolone formulation to be used in this clinical study is provided in Table 15.

TABLE 15
Clinical formulation of ER Brexanolone

| | Concentration (mg/mL)/w/v percentage |
|---|---|
| Composition | Clinical Formulation |
| Brexanolone | 100 and 300 mg/mL |
| PS 80 | 6.71/~0.67% |
| PEG 3350 | 6.71/~0.67% |
| Mannitol | 33.54/~3.4% |
| Sodium Citrate dihydrate | 1.72/~0.17% |
| Citric acid monohydrate | 0.104/~0.010% |

Abbreviations:
GLP = Good Laboratory Practice;
w/v = weight by volume

Control Product: Placebo (aqueous solution containing excipients without ER Brexanolone product)

### Study Procedures

Duration of Study:

The estimated total duration for each subject is up to 20 weeks, including screening period (4 weeks), dosing period (1 day), postdose PPD preventative treatment period (16 weeks).

Screening Period (Up to 4 Weeks):

Screening will be performed no more than 4 weeks prior to dosing (Day 1) and will include written informed consent, determination of eligibility, collection of demographics and medical history, full physical examination (including vitals), laboratory tests, screening viral serology, urinalysis, 12-lead ECG, Sheehan Suicidality Tracking Scale (S-STS; self-report) and other assessments per the protocol. Adverse events related to screening activities must be collected from the time of consent onwards; any other events occurring during the screening period should be reported as medical history. All SAEs must be collected from the time of consent onwards.

Dosing Period (1 Day; Day 1):

Dosing will be inpatient to enable safety and tolerability assessments. Eligible subjects will be randomized to receive a single dose of ER Brexanolone or placebo according to the dosing schedule. Safety assessments performed on Day 1 include vital signs, continuous pulse oximetry, and review of AEs and concomitant medication. See the full protocol for a complete listing of safety and efficacy assessments and timepoints.

Postdose PPD Preventative Treatment Period (16 Weeks):

### Primary Endpoints

Percentage of subjects with an occurrence of a PPD episode during the 16-week PPD preventive treatment period (see Study Design section for PPD episode criteria).

### Statistical Analysis

The percentage of subjects with an occurrence of a PPD episode during the 16-week PPD preventive treatment period for each treatment group will be reported.

Descriptive statistics will be presented for continuous variables, and frequencies and percentages will be presented for categorical and ordinal variables. Percentages will be based on the number of non-missing values in a treatment group. Details will be provided in the Statistical Analysis Plan.

### Additional Embodiments

1. An aqueous suspension pharmaceutical composition comprising a pharmaceutically effective amount of a neuroactive steroid selected from the group consisting of brexanolone, pharmaceutically acceptable salts and derivatives thereof, wherein the neuroactive steroid provides a therapeutically effective plasma concentration over a period of at least about 72 hours to treat a neurological condition when administered in one or more injections to a subject in need thereof.
2. The aqueous suspension pharmaceutical composition of embodiment 1, wherein the neuroactive steroid comprises brexanolone.
3. The aqueous suspension pharmaceutical composition of embodiment 1 or 2, comprising from 30 mg to 1000 mg of brexanolone.
4. The aqueous suspension pharmaceutical composition of any one of embodiments 1-3, wherein the concentration of brexanolone is from about 30 mg/mL to about 500 mg/mL.
5. The aqueous suspension pharmaceutical composition of any one of embodiments 1-4, wherein the brexanolone has a particle size distribution (PSD) with a Dv50 of from about 1 μm to about 5 μm.
6. The aqueous suspension pharmaceutical composition of embodiment 5, wherein the brexanolone has a particle size distribution (PSD) with a Dv50 of about 3 μm.
7. The aqueous suspension pharmaceutical composition of any one of embodiments 1-6, wherein the brexanolone has a particle size distribution (PSD) with a Dv90 of from about 4 μm to about 8 μm.
8. The aqueous suspension pharmaceutical composition of embodiment 7, wherein the brexanolone has a particle size distribution (PSD) with a Dv90 of about 6 μm.
9. The aqueous suspension pharmaceutical composition of any one of embodiments 1-8, further comprising one or more pharmaceutically acceptable excipients.
10. The aqueous suspension pharmaceutical composition of embodiment 9, wherein the one or more pharmaceutically acceptable excipients comprises a surfactant, a buffering agent, or both.
11. The aqueous suspension pharmaceutical composition of embodiment 10, wherein the surfactant is a nonionic surfactant.
12. The aqueous suspension pharmaceutical composition of embodiment 10 or 11, wherein the surfactant comprises polysorbate 80.
13. The aqueous suspension pharmaceutical composition of any one of embodiments 10-12, wherein the surfactant comprises about 0.2% to about 1.0% w/v of the composition.
14. The aqueous suspension pharmaceutical composition of embodiment 13, wherein the surfactant comprises about 0.5% to about 0.9% w/v of the composition.
15. The aqueous suspension pharmaceutical composition of embodiment 14, wherein the surfactant comprises about 0.6% to about 0.8% w/v of the composition.
16. The aqueous suspension pharmaceutical composition of any one of embodiments 10-15, wherein the buffering agent comprises about 0.1% to about 0.5% w/v of the composition.
17. The aqueous suspension pharmaceutical composition of any one of embodiments 10-16, wherein the buffering agent comprises a citrate buffering agent.
18. The aqueous suspension pharmaceutical composition of embodiment 17, wherein the citrate buffering agent comprises sodium citrate dihydrate and citric acid monohydrate.
19. The aqueous suspension pharmaceutical composition of embodiment 18, wherein the sodium citrate dihydrate is about 0.15% to about 0.2% w/v of the composition.
20. The aqueous suspension pharmaceutical composition of embodiment 18, wherein the citric acid monohydrate is about 0.010% to about 0.015% w/v of the composition.
21. The aqueous suspension pharmaceutical composition of any one of embodiments 1-20, further comprising a suspending agent.
22. The aqueous suspension pharmaceutical composition of embodiment 21, wherein the suspending agent comprises polyethylene glycol (PEG).
23. The aqueous suspension pharmaceutical composition of embodiment 22, wherein the PEG is a higher molecular weight PEG.
24. The aqueous suspension pharmaceutical composition of embodiment 23, wherein the higher molecular weight PEG is PEG 3350, PEG 4000 or PEG 6000.
25. The aqueous suspension pharmaceutical composition of embodiment 24, wherein the higher molecular weight PEG is PEG 3350.
26. The aqueous suspension pharmaceutical composition of any one of embodiments 21-25, wherein the suspending agent comprises about 0.2% to about 1.0% w/v of the composition.
27. The aqueous suspension pharmaceutical composition of embodiment 26, wherein the suspending agent comprises about 0.5% to about 0.9% w/v of the composition.
28. The aqueous suspension pharmaceutical composition of embodiment 27, wherein the suspending agent comprises about 0.6% to about 0.8% w/v of the composition.
29. The aqueous suspension pharmaceutical composition of any one of embodiments 1-28, further comprising atonicity adjusting agent.
30. The aqueous suspension pharmaceutical composition of embodiment 29, wherein the tonicity adjusting agent is selected from the group consisting of dextrose, mannitol and glycerin.
31. The aqueous suspension pharmaceutical composition of embodiment 30, wherein the tonicity adjusting agent is mannitol.
32. The aqueous suspension pharmaceutical composition of any one of embodiments 29-31, wherein the tonicity adjusting agent comprises about 2% to about 6% w/v of the pharmaceutical composition.
33. The aqueous suspension pharmaceutical composition of embodiment 32, wherein the tonicity adjusting agent comprises about 3% to about 4% w/v of the pharmaceutical composition.
34. The aqueous suspension pharmaceutical composition of any one of embodiments 1-33, wherein the pharmaceutical composition is substantially free of cyclodextrins.
35. The aqueous suspension pharmaceutical composition of embodiment 34, wherein the aqueous suspension pharmaceutical composition is substantially free of sulfobutyl ether β-cyclodextrin.
36. The aqueous suspension pharmaceutical composition of embodiment 34 or 35, wherein the pharmaceutical composition is substantially free of hydroxypropyl-β-cyclodextrin (HPBCD).
37. The aqueous suspension pharmaceutical composition of any one of embodiments 1-36, wherein the neuroactive steroid comprises a brexanolone crystalline form (polymorph Form A) characterized by having at least 2 of the following peaks in Powder X-Ray Diffraction (PXRD) diffractograms, at 7.25, 8.88, 11.46, 14.50, 14.78, 17.77, 18.15, 18.32, 18.61 and 19.99±0.1 2θ(°).
38. The aqueous suspension pharmaceutical composition of any one of embodiments 1-37, wherein the pharmaceutical composition provides a maximum blood plasma concentration (Cₘₐₓ) of more than about 10 ng/mL brexanolone following the one or more injections.
39. The aqueous suspension pharmaceutical composition of embodiment 38, wherein the maximum blood plasma concentration (Cₘₐₓ) of brexanolone ranges from about 20 ng/mL to about 80 ng/mL following the one or more injections.
40. The aqueous suspension pharmaceutical composition of embodiment 39, wherein the maximum blood plasma concentration (Cₘₐₓ) of brexanolone is about 50 ng/mL following the one or more injections.
41. The aqueous suspension pharmaceutical composition of any one of embodiments 1-40, wherein the maximum blood plasma concentration (Cₘₐₓ) of brexanolone following the one or more injections is less than 90% of the Cₘₐₓ of a reference product administered via IV infusion containing substantially the same amount of brexanolone.
42. The aqueous suspension pharmaceutical composition of any one of embodiments 1-41, wherein at least about 50% of the maximum blood plasma concentration (Cₘₐₓ) is maintained for a period greater than about 50 hours following the one or more injections.
43. The aqueous suspension pharmaceutical composition of any one of embodiments 1-41, wherein at least about 40% of the maximum blood plasma concentration (Cₘₐₓ) is maintained for a period greater than about 100 hours following the one or more injections.
44. The aqueous suspension pharmaceutical composition of any one of embodiments 1-41, wherein at least about 30% of the maximum blood plasma concentration (Cₘₐₓ) is maintained for a period greater than about 300 hours following the one or more injections.
45. The aqueous suspension pharmaceutical composition of any one of embodiments 1-44, wherein the pharmaceutical composition provides a mean steady state exposure (Cₛₛ) of brexanolone following the one or more injections within the range of about 80% to about 125% of the mean steady state exposure of a reference product administered via IV infusion containing substantially the same amount of brexanolone.
46. The aqueous suspension pharmaceutical composition of any one of embodiments 1-45, wherein the pharmaceutical composition provides a mean steady state exposure of brexanolone within the range of about 80% to about 125% of 52 ng/mL to about 79 ng/mL following the one or more injections.
47. The aqueous suspension pharmaceutical composition of any one of embodiments 1-46, wherein the pharmaceutical composition provides an average daily AUC of brexanolone that is at least about 50 ng*h/mL/day for at least about 72 hours following the one or more injections.
48. The aqueous suspension pharmaceutical composition of any one of embodiments 1-47, wherein the composition achieves a mean terminal elimination half-life (T_{1/2}) of brexanolone of greater than about 9 h following the one or more injections.
49. The aqueous suspension pharmaceutical composition of any one of embodiments 1-48, wherein the composition achieves a mean terminal elimination half-life (T_{1/2}) of brexanolone that is greater than the T_{1/2} of a reference product administered via IV infusion containing substantially the same amount of brexanolone.
50. A method, comprising administering to a subject in need thereof a therapeutically effective dose of the pharmaceutical composition of any one of embodiments 1-49.
51. A method of treating or preventing a neurological condition in a subject in need thereof, comprising administering to the subject a therapeutically effective dose of the pharmaceutical composition of any one embodiments 1-49.
52. The method of embodiment 50 or 51, wherein the pharmaceutical composition is administered to the subject between a pre-admin breastfeeding and a consecutive post-admin breastfeeding of the subject.
53. The method of any one of embodiments 50-52, wherein the pharmaceutical composition is administered to the subject from 1 minute to about 360 minutes after completion of the pre-admin breastfeeding.
54. The method of any one of embodiments 50-53, wherein the pharmaceutical composition is administered to the subject about 5 minutes to about 360 minutes before starting the post-admin breastfeeding.
55. The method of any one of embodiments 50-54, wherein the subject is a woman 1 day to 12 months after giving birth to a child.
56. The method of any one of embodiments 50-55, wherein the subject has not been diagnosed with the neurological condition at the time of administering the pharmaceutical composition.
57. The method of any one of embodiments 50-56, wherein the subject is diagnosed with the neurological condition within 2 years prior to administering the pharmaceutical composition.
58. The method of any one of embodiments 50-57, wherein the subject is diagnosed with the neurological condition during pregnancy prior to administering the pharmaceutical composition.
59. The method of any one of embodiments 50-58, wherein the subject has a family history of the neurological condition at the time of administering the pharmaceutical composition.
60. The method of any one of embodiments 50-59, wherein the neurological condition is selected from the group consisting of traumatic brain injury, Alzheimer's disease, mild cognitive impairment (MCI), epilepsy, seizures, anxiety, fragile X tremor-ataxia syndrome, lysosomal storage disorders (Niemann-Pick type C disease), post-traumatic stress disorder (PTSD), postpartum depression (PPD), major depressive disorder (MDD), premenstrual dysphoric disorder (PMDD), persistent depressive disorder (PDD), bipolar disorder, seasonal affective disorder (SAD), secondary depression, postfinasteride syndrome, alcohol craving, and smoking cessation.
61. The method of embodiment 60, wherein the neurological condition is postpartum depression (PPD).
62. The method of any one of embodiments 50-61, wherein the pharmaceutical composition is administered to the subject via intramuscular (IM) injection.
63. The method of any one of embodiments 50-62, wherein the subject experiences a reduction of depression that is characterized by at least a four point decline in total Hamilton Depression Rating Scale (HAM-D) value or by at least a two point decline in Montgomery Asberg Depression Rating Scale (MADRS) value, within two months after administering an initial dose of the pharmaceutical composition.
64. The method of any one of embodiments 50-63, wherein the subject experiences a reduction of depression that is characterized by at least a 40% reduction in HAM-D or MADRS value, within two months after administering an initial dose of the pharmaceutical composition.
65. The method of any one of embodiments 50-64, wherein the subject experiences a reduction of depression that is characterized by HAM-D or MADRS remission, within two months after administering an initial dose of the pharmaceutical composition.
66. The method of any one of embodiments 50-65, wherein the subject experiences a reduction of depression that is characterized by an at least two category change in HAM-D severity classification, within two months after administering an initial dose of the pharmaceutical composition.
67. The method of any one of embodiments 50-66, wherein the subject experiences a reduction of depression that is characterized by at least one point decline in one or more of the Clinical Global Impression (CGI) subscale scores, wherein the CGI subscales are selected from Severity of Illness Subscale (CGI-S) or Global Improvement Subscale (CGI-I), within two months after administering an initial dose of the pharmaceutical composition, within two months after administering an initial dose of the pharmaceutical composition.
68. The method of any one of embodiments 50-67, wherein the subject experiences a reduction of depression that is characterized by at least about a 10%, 20%, or 30% improvement in Symptoms of Depression Questionnaire (SDQ) total scale score or in any of the respective subscales of SDQ-1, SDQ-2, SDQ-3, SDQ-4 and SDQ-5, within two months after administering an initial dose of the pharmaceutical composition.
69. The method of any one of embodiments 50-68, wherein after administering an initial dose, the subject experiences a reduction of depression that is characterized by an at least one point decline in Pittsburgh Sleep Quality Index (PSQI) Global score.
70. The method of any one of embodiments 50-69, wherein the administering comprises:
   (a) administering an initial dose of the pharmaceutical composition of any one of embodiments 1-49; and
   (b) optionally, administering a second dose or subsequent dose of the pharmaceutical composition of any one of embodiments 1-49,
   wherein the second dose or subsequent doses are administered at a timepoint deemed necessary to maintain a therapeutically effective plasma concentration of brexanolone.
71. The method of embodiment 70, wherein the initial dose of brexanolone and subsequent dose(s) are the same.
72. The method of embodiment 70, wherein the initial dose of brexanolone and subsequent dose(s) are different.
73. The method of embodiment 72, wherein the initial dose of brexanolone is greater than a subsequent dose.
74. The method of embodiment 72, wherein the initial dose of brexanolone is less than a subsequent dose.
75. A method of preventing postpartum depression (PPD) in a subject in need thereof, said method comprising: 1) obtaining or causing to obtain depression assessment data of the subject, wherein the depression assessment data comprise depression diagnostic data and pregnancy data of the subject; 2) producing risk prediction data based on the depression assessment data; and 3) administering an aqueous suspension pharmaceutical composition comprising a pharmaceutically effective amount of a neuroactive steroid selected from the group consisting of brexanolone, pharmaceutically acceptable salts and derivatives thereof to the subject prior to clinical onset of the PPD if the risk prediction data indicate a high risk of PPD in the subject, wherein the neuroactive steroid provides a therapeutically effective plasma concentration over a period of at least about 72 hours to treat a neurological condition when administered in one or more injections to a subject in need thereof; and wherein the subject is not diagnosed with PPD at the time the depression assessment data is obtained.
76. The method of embodiment 75, wherein the aqueous suspension pharmaceutical composition is the aqueous suspension pharmaceutical composition of any one of embodiments 1-49.
77. The method of embodiment 75 or 76, wherein the depression diagnostic data comprise historic depression diagnostic data if any, depression data from previous pregnancy if any, present depression diagnostic data, historic Beck's Depression Inventory (BDI) value, present BDI value, historic Edinburgh Postnatal Depression Scale (EPDS) value, present EPDS value, historic Postpartum Depression Predictors Inventory (PDPI), present PDPI value, historic SIGH-ADS29 assessment value, present SIGH-ADS29 assessment value, historic Structured Clinical Interview for DSM-IV (SCID) assessment, present SCID assessment, historic Inventory of Depressive Symptomatology (IDS) assessment, present IDS assessment, historic Quick Inventory of Depressive Symptomatology (QIDS) assessment, present QIDS assessment, clinician IDS (IDS-C), clinician QIDS (QIDS-C), patient self-rated IDS (IDS-SR), patient self-rated QIDS (QIDS-SR), of said subject, or a combination thereof
78. The method of any one of embodiments 75-77, wherein the depression assessment data is obtained or caused to be obtained during pregnancy, in a range of from 10 weeks to 0 day prior to the completion of pregnancy, in a range of from 0 day to 24 weeks after completion of pregnancy, of the subject, or a combination thereof
79. The method of any one of embodiments 75-78, wherein the neuroactive steroid is administered to the subject in a range of from 0 day to 24 weeks after completion of pregnancy of the subject.
80. Use of the pharmaceutical composition of any one of embodiments 1-49 for manufacturing a medicament for treating or preventing a neurological condition.
81. Use of embodiment 80, wherein the neurological condition is selected from the group consisting of traumatic brain injury, Alzheimer's disease, mild cognitive impairment (MCI), epilepsy, seizures, anxiety, fragile X tremor-ataxia syndrome, lysosomal storage disorders (Niemann-Pick type C disease), post-traumatic stress disorder (PTSD), postpartum depression (PPD), major depressive disorder (MDD), premenstrual dysphoric disorder (PMDD), persistent depressive disorder (PDD), bipolar disorder, seasonal affective disorder (SAD), secondary depression, postfinasteride syndrome, alcohol craving, and smoking cessation.
82. Use of embodiment 81, wherein the neurological condition is postpartum depression (PPD).

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary, to employ concepts of the various patents, application and publications to provide yet further embodiments.

1. An aqueous suspension pharmaceutical composition comprising a pharmaceutically effective amount of a neuroactive steroid selected from the group consisting of brexanolone, and pharmaceutically acceptable salts and derivatives thereof, wherein the neuroactive steroid provides a therapeutically effective plasma concentration over a period of at least about 72 hours to treat a neurological condition when administered in one or more injections to a subject in need thereof.

2. The aqueous suspension pharmaceutical composition of claim 1, wherein the neuroactive steroid comprises brexanolone.

3. The aqueous suspension pharmaceutical composition of claim 2, comprising from 30 mg to 1000 mg of brexanolone.

4. The aqueous suspension pharmaceutical composition of claim 2, wherein the concentration of brexanolone is from about 30 mg/mL to about 500 mg/mL.

5. The aqueous suspension pharmaceutical composition of claim 2, wherein the brexanolone has a particle size distribution (PSD) with a Dv50 of from about 1 μm to about 5 μm.

6. The aqueous suspension pharmaceutical composition of claim 5, wherein the brexanolone has a particle size distribution (PSD) with a Dv50 of about 3 μm.

7. The aqueous suspension pharmaceutical composition of claim 2, wherein the brexanolone has a particle size distribution (PSD) with a Dv90 of from about 4 μm to about 8 μm.

8. The aqueous suspension pharmaceutical composition of claim 7, wherein the brexanolone has a particle size distribution (PSD) with a Dv90 of about 6 μm.

9. The aqueous suspension pharmaceutical composition of claim 1, further comprising one or more pharmaceutically acceptable excipients.

10. The aqueous suspension pharmaceutical composition of claim 9, wherein the one or more pharmaceutically acceptable excipients comprises a surfactant, a buffering agent, or both.

11. The aqueous suspension pharmaceutical composition of claim 10, wherein the surfactant is a nonionic surfactant.

12. The aqueous suspension pharmaceutical composition of claim 10, wherein the surfactant comprises polysorbate 80.

13. The aqueous suspension pharmaceutical composition of claim 10, wherein the surfactant comprises about 0.2% to about 1.0% w/v of the composition.

14. The aqueous suspension pharmaceutical composition of claim 13, wherein the surfactant comprises about 0.5% to about 0.9% w/v of the composition.

15. The aqueous suspension pharmaceutical composition of claim 14, wherein the surfactant comprises about 0.6% to about 0.8% w/v of the composition.

16. The aqueous suspension pharmaceutical composition of claim 10, wherein the buffering agent comprises about 0.1% to about 0.5% w/v of the composition.

17. The aqueous suspension pharmaceutical composition of claim 10, wherein the buffering agent comprises a citrate buffering agent.

18. The aqueous suspension pharmaceutical composition of claim 17, wherein the citrate buffering agent comprises sodium citrate dihydrate and citric acid monohydrate.

19. The aqueous suspension pharmaceutical composition of claim 18, wherein the sodium citrate dihydrate is about 0.15% to about 0.2% w/v of the composition.

20. The aqueous suspension pharmaceutical composition of claim 18, wherein the citric acid monohydrate is about 0.010% to about 0.015% w/v of the composition.

21. The aqueous suspension pharmaceutical composition of claim 1, further comprising a suspending agent.

22. The aqueous suspension pharmaceutical composition of claim 21, wherein the suspending agent comprises polyethylene glycol (PEG).

23. The aqueous suspension pharmaceutical composition of claim 22, wherein the PEG is a higher molecular weight PEG.

24. The aqueous suspension pharmaceutical composition of claim 23, wherein the higher molecular weight PEG is PEG 3350, PEG 4000 or PEG 6000.

25. The aqueous suspension pharmaceutical composition of claim 24, wherein the higher molecular weight PEG is PEG 3350.

26. The aqueous suspension pharmaceutical composition of claim 21, wherein the suspending agent comprises about 0.2% to about 1.0% w/v of the composition.

27. The aqueous suspension pharmaceutical composition of claim 26, wherein the suspending agent comprises about 0.5% to about 0.9% w/v of the composition.

28. The aqueous suspension pharmaceutical composition of claim 27, wherein the suspending agent comprises about 0.6% to about 0.8% w/v of the composition.

29. The aqueous suspension pharmaceutical composition of claim 1, further comprising a tonicity adjusting agent.

30. The aqueous suspension pharmaceutical composition of claim 29, wherein the tonicity adjusting agent is selected from the group consisting of dextrose, mannitol and gly cerin.

31. The aqueous suspension pharmaceutical composition of claim 30, wherein the tonicity adjusting agent is mannitol.

32. The aqueous suspension pharmaceutical composition of claim 29, wherein the tonicity adjusting agent comprises about 2% to about 6% w/v of the pharmaceutical composition.

33. The aqueous suspension pharmaceutical composition of claim 32, wherein the tonicity adjusting agent comprises about 3% to about 4% w/v of the pharmaceutical composition.

34. The aqueous suspension pharmaceutical composition of claim 1, wherein the pharmaceutical composition is substantially free of cyclodextrins.

35. The aqueous suspension pharmaceutical composition of claim 34, wherein the aqueous suspension pharmaceutical composition is substantially free of sulfobutyl ether β-cyclodextrin.

36. The aqueous suspension pharmaceutical composition of claim 34, wherein the pharmaceutical composition is substantially free of hydroxypropyl-β-cyclodextrin (HPBCD).

37. The aqueous suspension pharmaceutical composition of claim 1, wherein the neuroactive steroid comprises a brexanolone crystalline form (polymorph Form A) characterized by having at least 2 of the following peaks in Powder X-Ray Diffraction (PXRD) diffractograms, at 7.25, 8.88, 11.46, 14.50, 14.78, 17.77, 18.15, 18.32, 18.61 and 19.99±0.1 2θ(°).

38. The aqueous suspension pharmaceutical composition of claim 2, wherein the pharmaceutical composition provides a maximum blood plasma concentration (Cₘₐₓ) of more than about 10 ng/mL brexanolone following the one or more injections.

39. The aqueous suspension pharmaceutical composition of claim 38, wherein the maximum blood plasma concentration (Cₘₐₓ) of brexanolone ranges from about 20 ng/mL to about 80 ng/mL following the one or more injections.

40. The aqueous suspension pharmaceutical composition of claim 39, wherein the maximum blood plasma concentration (Cₘₐₓ) of brexanolone is about 50 ng/mL following the one or more injections.

41. The aqueous suspension pharmaceutical composition of claim 2, wherein the maximum blood plasma concentration (Cₘₐₓ) of brexanolone following the one or more injections is less than 90% of the Cₘₐₓ of a reference product administered via IV infusion containing substantially the same amount of brexanolone.

42. The aqueous suspension pharmaceutical composition of claim 2, wherein at least about 50% of the maximum blood plasma concentration (Cₘₐₓ) is maintained for a period greater than about 50 hours following the one or more injections.

43. The aqueous suspension pharmaceutical composition of claim 2, wherein at least about 40% of the maximum blood plasma concentration (Cₘₐₓ) is maintained for a period greater than about 100 hours following the one or more injections.

44. The aqueous suspension pharmaceutical composition of claim 2, wherein at least about 30% of the maximum blood plasma concentration (Cₘₐₓ) is maintained for a period greater than about 300 hours following the one or more injections.

45. The aqueous suspension pharmaceutical composition of claim 2, wherein the pharmaceutical composition provides a mean steady state exposure (Cₛₛ) of brexanolone following the one or more injections within the range of about 80% to about 125% of the mean steady state exposure of a reference product administered via IV infusion containing substantially the same amount of brexanolone.

46. The aqueous suspension pharmaceutical composition of claim 2, wherein the pharmaceutical composition provides a mean steady state exposure of brexanolone within the range of about 80% to about 125% of 52 ng/mL to about 79 ng/mL following the one or more injections.

47. The aqueous suspension pharmaceutical composition of claim 2, wherein the pharmaceutical composition provides an average daily AUC of brexanolone that is at least about 50 ng*h/mL/day for at least about 72 hours following the one or more injections.

48. The aqueous suspension pharmaceutical composition of claim 2, wherein the composition achieves a mean terminal elimination half-life (T_{1/2}) of brexanolone of greater than about 9 h following the one or more injections.

49. The aqueous suspension pharmaceutical composition of claim 2, wherein the composition achieves a mean terminal elimination half-life (T_{1/2}) of brexanolone that is greater than the T_{1/2} of a reference product administered via IV infusion containing substantially the same amount of brexanolone.

50. A method, comprising administering to a subject in need thereof a therapeutically effective dose of the pharmaceutical composition of any one of claims 1-49.

51. A method of treating or preventing a neurological condition in a subject in need thereof, comprising administering to the subject a therapeutically effective dose of the pharmaceutical composition of any one claims 1-49.

52. A method of treating or preventing a neurological condition in a subject in need thereof, comprising administering to the subject an aqueous suspension pharmaceutical composition comprising a pharmaceutically effective amount of a neuroactive steroid selected from the group consisting of brexanolone, pharmaceutically acceptable salts and derivatives thereof, wherein the neuroactive steroid provides a therapeutically effective plasma concentration over a period of at least about 72 hours.

53. The method of claim 52, wherein the pharmaceutical composition is administered to the subject between a pre-admin breastfeeding and a consecutive post-admin breastfeeding of the subject.

54. The method of claim 52, wherein the pharmaceutical composition is administered to the subject from 1 minute to about 360 minutes after completion of the pre-admin breastfeeding.

55. The method of claim 52, wherein the pharmaceutical composition is administered to the subject about 5 minutes to about 360 minutes before starting the post-admin breastfeeding.

56. The method of claim 52, wherein the subject is a woman 1 day to 12 months after giving birth to a child.

57. The method of claim 52, wherein the subject has not been diagnosed with the neurological condition at the time of administering the pharmaceutical composition.

58. The method of claim 52, wherein the subject is diagnosed with the neurological condition within 2 years prior to administering the pharmaceutical composition.

59. The method of claim 52, wherein the subject is diagnosed with the neurological condition during pregnancy prior to administering the pharmaceutical composition.

60. The method of claim 52, wherein the subject has a family history of the neurological condition at the time of administering the pharmaceutical composition.

61. The method of claim 52, wherein the neurological condition is selected from the group consisting of traumatic brain injury, Alzheimer's disease, mild cognitive impairment (MCI), epilepsy, seizures, anxiety, fragile X tremor-ataxia syndrome, lysosomal storage disorders (Niemann-Pick type C disease), post-traumatic stress disorder (PTSD), postpartum depression (PPD), major depressive disorder (MDD), premenstrual dysphoric disorder (PMDD), persistent depressive disorder (PDD), bipolar disorder, seasonal affective disorder (SAD), secondary depression, postfinasteride syndrome, alcohol craving, and smoking cessation.

62. The method of claim 61, wherein the neurological condition is postpartum depression (PPD).

63. The method of claim 52, wherein the pharmaceutical composition is administered to the subject via intramuscular (IM) injection.

64. The method of claim 52, wherein the subject experiences a reduction of depression that is characterized by at least a four point decline in total Hamilton Depression Rating Scale (HAM-D) value or by at least a two point decline in Montgomery Asberg Depression Rating Scale (MADRS) value, within two months after administering an initial dose of the pharmaceutical composition.

65. The method of claim 52, wherein the subject experiences a reduction of depression that is characterized by at least a 40% reduction in HAM-D or MADRS value, within two months after administering an initial dose of the pharmaceutical composition.

66. The method of claim 52, wherein the subject experiences a reduction of depression that is characterized by HAM-D or MADRS remission, within two months after administering an initial dose of the pharmaceutical composition.

67. The method of claim 52, wherein the subject experiences a reduction of depression that is characterized by an at least two category change in HAM-D severity classification, within two months after administering an initial dose of the pharmaceutical composition.

68. The method of claim 52, wherein the subject experiences a reduction of depression that is characterized by at least one point decline in one or more of the Clinical Global Impression (CGI) subscale scores, wherein the CGI subscales are selected from Severity of Illness Subscale (CGI-S) or Global Improvement Subscale (CGI-I), within two months after administering an initial dose of the pharmaceutical composition, within two months after administering an initial dose of the pharmaceutical composition.

69. The method of claim 52, wherein the subject experiences a reduction of depression that is characterized by at least about a 10%, 20%, or 30% improvement in Symptoms of Depression Questionnaire (SDQ) total scale score or in any of the respective subscales of SDQ-1, SDQ-2, SDQ-3, SDQ-4 and SDQ-5, within two months after administering an initial dose of the pharmaceutical composition.

70. The method of claim 52, wherein after administering an initial dose, the subject experiences a reduction of depression that is characterized by an at least one point decline in Pittsburgh Sleep Quality Index (PSQI) Global score.

71. The method of claim 52, wherein the administering comprises:
(a) administering an initial dose of the pharmaceutical composition of any one of claims 1-49; and
(b) optionally, administering a second dose or subsequent dose of the pharmaceutical composition of any one of claims 1-49,
wherein the second dose or subsequent doses are administered at a timepoint deemed necessary to maintain a therapeutically effective plasma concentration of brexanolone.

72. The method of claim 71, wherein the initial dose of brexanolone and subsequent dose(s) are the same.

73. The method of claim 71, wherein the initial dose of brexanolone and subsequent dose(s) are different.

74. The method of claim 73, wherein the initial dose of brexanolone is greater than a subsequent dose.

75. The method of claim 73, wherein the initial dose of brexanolone is less than a subsequent dose.

76. A method of preventing postpartum depression (PPD) in a subject in need thereof, said method comprising: 1) obtaining or causing to obtain depression assessment data of the subject, wherein the depression assessment data comprise depression diagnostic data and pregnancy data of the subject; 2) producing risk prediction data based on the depression assessment data; and 3) administering an aqueous suspension pharmaceutical composition comprising a pharmaceutically effective amount of a neuroactive steroid selected from the group consisting of brexanolone, pharmaceutically acceptable salts and derivatives thereof to the subject prior to clinical onset of the PPD if the risk prediction data indicate a high risk of PPD in the subject, wherein the neuroactive steroid provides a therapeutically effective plasma concentration over a period of at least about 72 hours to treat a neurological condition when administered in one or more injections to a subject in need thereof and wherein the subject is not diagnosed with PPD at the time the depression assessment data is obtained.

77. The method of claim 76, wherein the aqueous suspension pharmaceutical composition is the aqueous suspension pharmaceutical composition of any one of claims 1-49.

78. The method of claim 76, wherein the depression diagnostic data comprise historic depression diagnostic data if any, depression data from previous pregnancy if any, present depression diagnostic data, historic Beck's Depression Inventory (BDI) value, present BDI value, historic Edinburgh Postnatal Depression Scale (EPDS) value, present EPDS value, historic Postpartum Depression Predictors Inventory (PDPI), present PDPI value, historic SIGH-ADS29 assessment value, present SIGH-ADS29 assessment value, historic Structured Clinical Interview for DSM-IV (SCID) assessment, present SCID assessment, historic Inventory of Depressive Symptomatology (IDS) assessment, present IDS assessment, historic Quick Inventory of Depressive Symptomatology (QIDS) assessment, present QIDS assessment, clinician IDS (IDS-C), clinician QIDS (QIDS-C), patient self-rated IDS (IDS-SR), patient self-rated QIDS (QIDS-SR), of said subject, or a combination thereof.

79. The method of claim 76, wherein the depression assessment data is obtained or caused to be obtained during pregnancy, in a range of from 10 weeks to 0 day prior to the completion of pregnancy, in a range of from 0 day to 24 weeks after completion of pregnancy, of the subject, or a combination thereof.

80. The method of claim 76, wherein the neuroactive steroid is administered to the subject in a range of from 0 day to 24 weeks after completion of pregnancy of the subject.

81. Use of the pharmaceutical composition of any one of claims 1-49 for manufacturing a medicament for treating or preventing a neurological condition.

82. Use of claim 81, wherein the neurological condition is selected from the group consisting of traumatic brain injury, Alzheimer's disease, mild cognitive impairment (MCI), epilepsy, seizures, anxiety, fragile X tremor-ataxia syndrome, lysosomal storage disorders (Niemann-Pick type C disease), post-traumatic stress disorder (PTSD), postpartum depression (PPD), major depressive disorder (MDD), premenstrual dysphoric disorder (PMDD), persistent depressive disorder (PDD), bipolar disorder, seasonal affective disorder (SAD), secondary depression, postfinasteride syndrome, alcohol craving, and smoking cessation.

83. Use of claim 82, wherein the neurological condition is postpartum depression (PPD).
